SEQUENCE LISTING (32 sequences)

SEQ ID NO: 1 - DNA, 322 bp, synthetic construct
  source 1..322: mol_type = other DNA
agcaaaagcaggtcaaatatattcaatatggaaagaataaaagaactaagagatttgatgtcacagtctcgcactcgcgagatactgacaaaaacaactgtggaccatatggccataatcgggccagcattgagcatcaacgaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 2 - DNA, 2341 bp, unidentified
  source 1..2341: mol_type = genomic DNA; note = Influenza virus subtype H7N9
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacacagtcaacagaacacataaatactcagaaaaaggaaagtggacaacgaacacagagactggagcaccccaactcaatccaattgatggaccattacccgaggacaacgagccgagtgggtatgcacaaacggattgtgtattggaagcaatggctttccttgaagaatctcacccagggatctttgaaaactcgtgtctcgaaacgatggaaattgttcagcaaacaagagtggataaactgacccaaggccgccagacctatgactggacgttgaatagaaatcagccggctgctaccgcattggccaacactatagaggtattcagatcgaatggcctgacagccaatgaatcaggaaggttgatcgatttcctcaaggacgtgatggattcaatggataaggaagaaatggagattacaacacatttccagaggaagaggagagtgagggacaacatgaccaagaaaatggtcacacagagaacaataggaaagaaaaaacaaagactgaacaaaaggagctacctaataagagcactgacattgaacacaatgacaaaggatgctgaaagaggcaagctgaaaaggagggcaatcgcaacacccgggatgcaaatcagaggattcgtgtattttgtagaagcactagcgaggagcatctgtgagaaacttgagcaatctggcctccctgtcggagggaatgagaagaaagctaaattggcaaatgttgtaaggaagatgatgactaattcacaagatacagagctctccttcacaattactggggacaacaccaaatggaatgagaatcaaaacccccggatgtttctagcaatgataacatacatcacaagaaaccagccagaatggtttagaaatgtcttaagcattgctcctataatgttctcaaacaagatggcgagattaggaaaagggtacatgttcgaaagtaagagtatgaagttacggacacaagtaccagcggaaatgctcgcaaatattgacctgaaatacttcaacaaatcaacaagagagaaaatcgagaaaataagacctctactgatagatggcacagcctcattgagtcctggaatgatgatgggcatgttcaacatgttgagtacagtcttaggagtttcaattctgaatctcgggcagaagaagtacaccaaaaccacatattggtgggacggactccaatcctcagatgacttcgccctcatagtgaatgcaccgaatcatgagggaatacaggcaggagtagataggttctatagaacctgcaaattagttgggataaacatgagcaagaagaaatcctacataaatcggacaggaacattcgaattcacaagctttttctaccgctatggattcgtagctaacttcagtatggagttgcccagttttggagtgtccgggattaatgagtcagctgacatgagcgttggtgttacagtaataaagaacaatatgataaacaacgatcttggaccagcaacagcccaaatggcccttcagctatttatcaaagactacagatacacataccgatgtcacaggggtgatacgcaaattcaaacgaggagagcattcgagctgaagaagctgtgggagcagacccgttcgaaggcaggactgttggtttcagatggagggccaaacctgtacaatatccggaacctccacattccagaggtctgcttgaaatgggaattgatggatgaagactaccaaggcaggttgtgtaatcctatgaacccgtttgtcagtcataaggaaattgattcagtcaacaatgctgtggtgatgccagctcatggcccagccaaaagcatggagtatgatgccgttgcaaccacacattcatggattcctaagaggaatcgctccattctcaacaccagccaaagggggattcttgaggacgaacagatgtaccagaagtgctgcaacctattcgaaaagttcttccccagcagttcgtacaggaggccagttggaatttccagcatggtggaggccatggtgtctagggcccgaattgatgcacgaattgacttcgaatctggaaggattaagaaagaagagttcgctgagatcatgaagatctgttccaccattgaagagctcagacggcaaaaatagtgaatttagcttgtccttcatgaaaaaatgccttgtttctact

SEQ ID NO: 3 - DNA, 2233 bp, unidentified
  source 1..2233: mol_type = genomic DNA; note = Influenza virus subtype H7N9
agcaaaagcaggtactgatccaaaatggaagactttgtgcgacaatgcttcaatccaatgatcgtcgagcttgcggaaaaggcaatgaaagaatatggggaagatccgaaaatcgaaacaaacaaattcgcatcaatatgcacacacttagaagtctgcttcatgtactctgatttccacttcatcgacgaacgaggcgaatcaactattatagaatctggcgatccaaatgtgctgctgaaacaccgatttgaaataatcgaagggagagaccgaacaatggcctggacagtggtgaatagtatctgcaacaccacgggagtcgaaaaacccaaatttctcccggatctgtatgactacaaggaaaaccgattcattgaaattggagtgacgaggagggaagtccacatatattacctagagaaagccaacaaaataaaatccgagaagacacacatccacattttctcattcactggagaagagatggccaccaaagcagattacactcttgacgaagaaagcagggcaagaatcaaaaccaggctgttcaccataaggcaggaaatggccagcaggggtctatgggattcctttcgtcagtctgaaagaggcgaagaaacaattgaagagagatttgaaatcacaggaaccatgcgcaggcttgccgaccaaagtctcccaccgaacttctccagccttgaaaactttagagcctatgtggatggattcgaaccgaacggctgcattgagggcaagctttctcaaatgtcaaaagaagtgaacgccagaatcgagccatttctaaggacaacaccacgccctctcagattgcctgatgggcctccctgctctcagcggtcgaaattcttgctgatggatgctctgaaattaagcattgaggacccgagccacgaaggggaggggataccgctatatgatgcgatcaaatgcatgaaaacgttcttcgggtggaaggagcccaacattatcaaaccacatgagaaaggcataaaccccaattatctcctgacttggaagcaggtgctagcagaacttcaggacattgaaaatgaagagaagattccaaggacaaagaacatgaagaaaacaagccaattaaagtgggcactcggtgagaacatggcaccggagaaggtggactttgaggattgcaaagatgtcaacgacctgaaacagtacgacagtgatgagccagagcccagatcactagcatgttggatccagagtgaattcaacaaggcatgtgaattgactgattcaagctgggtagaacttgatgaaataggggaagatgttgctccaatcgaacacattgcaagcatgagacggaactatttcacagcagaggtgtcccactgcagggctactgaatatataatgaagggagtgtacataaatacagctttgctcaatgcatcttgtgcagccatggatgactttcaactgatcccaatgataagtaaatgcagaaccaaagaaggaagacggaaaacaaacctatatggattcattataaaaggaaggtctcacttgaggaatgatactgacgtggtaaactttgtaagtatggaattttcccttaccgacccaaggttggaaccacataaatgggaaaagtattgtgttcttgaaataggggacatgctcctgcgaactgcagtaggccaagtgtctagacccatgtttctgtatgtgagaaccaatgggacctccaagatcaagatgaaatggggtatggaaatgagacgctgccttcttcaatctctccaacagattgagagcatgattgaagctgaatcctccgtcaaagagaaagacctgaccaaggaattctttgaaaacaaatctgaaacatggccaattggagaatcacccaaaggagtggaggaaggttccatcgggaaggtgtgcagaaccttactagcaaaatctgtattcaacagtctatatgcatctccgcaactcgaggggttctcagctgaatcgagaaaactgctactcattgttcaggcgcttagggataacctggaacctggaaccttcgatcttgaagggctatatgaagcaatcgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 4 - DNA, 1775 bp, unidentified
  source 1..1775: mol_type = genomic DNA; note = Influenza virus subtype H1N1
agcaaaagcaggggaaaataaaaacaaccaaaatgaaggcaaaactactggtcctgttatatgcatttgtagctacagatgcagacacaatatgtataggctaccatgcgaacaactcaaccgacactgttgacacaatactcgagaagaatgtggcagtgacacattctgttaacctgctcgaagacagccacaacgggaaactatgtaaattaaaaggaatagccccactacaattggggaaatgtaacatcaccggatggctcttgggaaatccagaatgcgactcactgcttccagcgagatcatggtcctacattgtagaaacaccaaactctgagaatggagcatgttatccaggagatctcatcgactatgaggaactgagggagcaattgagctcagtatcatcattagaaagattcgaaatatttcccaaggaaagttcatggcccaaccacacattcaacggagtaacagtatcatgctcccataggggaaaaagcagtttttacagaaatttgctatggctgacgaagaagggggattcatacccaaagctgaccaattcctatgtgaacaataaagggaaagaagtccttgtactatggggtgttcatcacccgtctagcagtgatgagcaacagagtctctatagtaatggaaatgcttatgtctctgtagcgtcttcaaattataacaggagattcaccccggaaatagctgcaaggcccaaagtaagagatcaacatgggaggatgaactattactggaccttgctagaacccggagacacaataatatttgaggcaactggtaatctaatagcaccatggtatgctttcgcactgagtagagggtttgagtccggcatcatcacctcaaacgcgtcaatgcatgagtgtaacacgaagtgtcaaacaccccagggatctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacaaaatgccattaacgggattacaaacaaggtgaactctgttatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatgaaagaactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctgagattaggatttcagaaatataaggaaaaacacccttgtttctact

SEQ ID NO: 5 - DNA, 1565 bp, unidentified
  source 1..1565: mol_type = genomic DNA; note = Influenza virus subtype H1N1
agcaaaagcagggtagataatcactcacagagtgacatcgaaatcatggcgaccaaaggcaccaaacgatcttacgaacagatggagactgatggagaacgccagaatgccactgaaatcagagcatctgtcggaaaaatgattgatggaattggacgattctacatccaaatgtgcaccgaacttaaactcagtgattatgagggacggctgattcagaacagcttaacaatagagagaatggtgctctctgcttttgacgagaggaggaataaatatctagaagaacatcccagtgcggggaaagatcctaagaaaactggaggacctatatacaggagagtagatggaaagtggaggagagaactcatcctttatgacaaagaagaaataagacgaatctggcgccaagctaataatggtgacgatgcaacggctggtctgactcacatgatgatctggcactccaatttgaatgatgcaacttaccagaggacaagagctcttgttcgcacaggaatggatcccaggatgtgctcactgatgcagggttcaaccctccctaggaggtctggggccgcaggtgctgcagtcaaaggagttggaacaatggtgatggaattgatcagaatgatcaaacgtgggatcaatgatcggaacttctggaggggtgagaatggacggagaacaaggattgcttatgaaagaatgtgcaacattctcaaagggaaatttcaaacagctgcacaaagaacaatggtggatcaagtgagagagagccggaatccaggaaatgctgagttcgaagatctcatctttttagcacggtctgcactcatattgagagggtcagttgctcacaagtcctgcctgcctgcctgtgtgtatggatctgccgtagccagtggatacgactttgaaagagagggatactctctagtcggaatagaccctttcagactgcttcaaaacagccaagtatacagcctaatcagaccaaatgagaatccagcacacaagagtcaactggtgtggatggcatgccattctgctgcatttgaagatctaagagtatcaagcttcatcagagggacgaaagtggtcccaagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaacctacgttctcagtacagagaaatctcccttttgacagaccaaccattatggcagcattcactgggaatacagaggggagaacatctgacatgagaaccgaaatcataaggctgatggaaagtgcaagaccagaagatgtgtctttccaggggcggggagtcttcgagctctcggacgaaaaggcaacgagcccgatcgtgccctcctttgacatgagtaatgaaggatcttatttcttcggagacaatgcagaggagtacgacaattaaagaaaaatacccttgtttctact

SEQ ID NO: 6 - DNA, 1409 bp, unidentified
  source 1..1409: mol_type = genomic DNA; note = Influenza virus subtype H1N1
agcgaaagcaggagtttaaatgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaagggggacctttaaggacagaagcccttatagggccttaatgagctgccctgtcggtgaagctccgtccccgtacaattcaaggtttgaatcggttgcttggtcagcaagtgcatgtcatgatggaatgggctggctaacaatcggaatttctggtccagatgatggagcagtggctgtattaaaatacaaccgcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcgatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggcaatggtgtttggataggaaggactaaaagtgacagttccagacatgggtttgagatgatttgggatcctaatggatggacagagactgatagtaggttctctatgagacaagatgttgtggcaataactaatcggtcagggtacagcggaagtttcgttcaacatcctgagctaacagggctagactgtatgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtttgttcaaaaaactccttgtttctact

SEQ ID NO: 7 - DNA, 1027 bp, unidentified
  source 1..1027: mol_type = genomic DNA; note = Influenza virus subtype H1N1
agcaaaagcaggtagatattgaaagatgagtcttctaaccgaggtcgaaacgtacgttctctctatcgtcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacaacaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccgttgggactcatcctagctccagtgctggtctaaaagatgatcttcttgaaaatttgcaggcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaaaaaactaccttgtttctact

SEQ ID NO: 8 - DNA, 890 bp, unidentified
  source 1..890: mol_type = genomic DNA; note = Influenza virus subtype H1N1
agcaaaagcagggtgacaaagacataatggatccaaacactgtgtcaagctttcaggtagattgctttctttggcatgtccgcaaaagagttgcagaccaagaactaggtgatgccccattccttgatcggcttcgccgagatcagaagtccctaagaggaagaggcagcactcttggtctggacatcgaaacagccacccgtgctggaaagcaaatagtggagcggattctgaaggaagaatctgatgaggcactcaaaatgaccatggcctctgtacctgcatcgcgctacctaactgacatgactcttgaggaaatgtcaaggcactggttcatgctcatgcccaagcagaaagtggcaggccctctttgtatcagaatggaccaggcgatcatggataagaacatcatactgaaagcgaacttcagtgtgatttttgaccggctggagactctaatattactaagggccttcaccgaagaggggacaattgttggcgaaatttcaccactgccctctcttccaggacatactgatgaggatgtcaaaaatgcagttggggtcctcatcggaggacttgaatggaataataacacagttcgagtctctgaaactctacagagattcgcttggagaagcagtaatgagaatgggagacctccactcactccaaaacagaaacggaaaatggcgggaacaattaggtcagaagtttgaagaaataagatggttgattgaagaagtgagacacagactgaagataacagagaatagttttgagcaaataacatttatgcaagccttacaactattgcttgaagtggagcaagagataagaactttctcgtttcagcttatttaataataaaaaacacccttgtttctact

SEQ ID NO: 9 - DNA, 546 bp, synthetic construct
  source 1..546: mol_type = other DNA
agcaaaagcaggtcaaatatattcaatatggaaagaataaaagaactaagagatttgatgtcacagtctcgcactcgcgagatactgacaaaaacaactgtggaccatatggccataatcaagaaatatacatcaggaagacaggagaagaatcctgcccttaggatgaagtggatgatggcaatgaaatatccaattacggcagacaaaaggataatggagatgatcccggaaagaaatgagcaaggtcagaccctttggagcaagacaaatgatgctggatcagacagagtgatggtgtcacctcgggatttctgattctgggcaaagaaaacaaaagatatgggccagcattgagcatcaacgaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 10 - DNA, 596 bp, synthetic construct
  source 1..596: mol_type = other DNA
agcaaaagcaggtcaaatatattcaatatggaaagaataaaagaactaagagatttgatgtcacagtctcgcactcgcgagatactgacaaaaacaactgtggaccatatggccataatcaagaaatatacatcaggaagacaggagaagaatcctgcccttaggatgaagtggatgatggcaatgaaatatccaattacggcagacaaaaggataatggagatgatcccggaaagaaatgagcaaggtcagaccctttggagcaaccaagaggcttacggtgcttggaaaggatgcaggtgcattgatggaagaccccgatgagggaacagcaggagtggaatctgcggtattgaggggatttctgattctgggcaaagaaaacaaaagatatgggccagcattgagcatcaacgaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 11 - DNA, 910 bp, synthetic construct
  source 1..910: mol_type = other DNA
agcaaaagcaggtcaaatatattcaatatggaaagaataaaagaactaagagatttgatgtcacagtctcgcactcgcgagatactgacaaaaacaactgtggaccatatggccataatcaagaaatatacatcaggaagacaggagaagaatcctgcccttaggatgaagtggatgatggcaatgaaatatccaattacggcagacaaaaggataatggagatgatcccggaaagaaatgagcaaggtcagaccctttggagcaagacaaatgatgctggatcagacagagtgatggtgtcacctctggctgtgacgtggtggaacagaaatggaccaacgacaagcacagtccattatccaaaggtctataaaacctattttgaaaaggtcgaaaggcagatgcgtgacgtactgggaacatttgacactgtccaaataataaagctattaccatttgcagcagccccgccggagcagagtaggatgcagttctcttctctaactgtgaatgtgaggggttccggaatgagaatagttgtgagaggcaattctcctgtgttcaactacaacaaggcaaccaagaggcttacggtgcttggaaaggatgcaggtgcattgatggaagaccccgatgagggaacagcaggagtggaatctgcggtattgaggggatttctgattctgggcaaagaaaacaaaagatatgggccagcattgagcatcaacgaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 12 - DNA, 458 bp, synthetic construct
  source 1..458: mol_type = other DNA
agcaaaagcaggtcaaatatattcaatatggaaagaataaaagaactaagagatttgatgtcacagtctcgcactcgcgagatactgacaaaaacaactgtggaccatatggccataatcaagaaatatacatcaggaagacaggagaagaatcctgcccttaggatgaagtggatgatggcaatgaaatatccaattacggcagacaaaaggataatggagatgatcccggaaagaaatgagcaaggtcagaccctttggagcaagacaaatgatggaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 13 - DNA, 548 bp, synthetic construct
  source 1..548: mol_type = other DNA
agcaaaagcaggtcaaatatattcaatatggaaagaataaaagaactaagagatttgatgtcacagtctcgcactcgcgagatactgacaaaaacaactgtggaccatatggccataatcaagaaatatacatcaggaagacaggagaagaatcctgcccttaggatgaagtggatgatggcaatgaaatatccaattacggcagacaaaaggataatggagatgatcccggaaagaaatgagcaaggtcagaccctttggagcaagacaaatgatgctggatcagacagagtgatggtgtcacctcaggggatttctgattctgggcaaagaaaacaaaagatatgggccagcattgagcatcaacgaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 14 - DNA, 597 bp, synthetic construct
  source 1..597: mol_type = other DNA
agcaaaagcaggtcaaatatattcaatatggaaagaataaaagaactaagagatttgatgtcacagtctcgcactcgcgagatactgacaaaaacaactgtggaccatatggccataatcaagaaatatacatcaggaagacaggagaagaatcctgcccttaggatgaagtggatgatggcaatgaaatatccaattacggcagacaaaaggataatggagatgatcccggaaagaaatgagctgtgttcaactacaacaaggcaaccaagaggcttacggtgcttggaaaggatgcaggtgcattgatggaagaccccgatgagggaacagcaggagtggaatctgcggtattgaggggatttctgattctgggcaaagaaaacaaaagatatgggccagcattgagcatcaacgaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 15 - DNA, 751 bp, synthetic construct
  source 1..751: mol_type = other DNA
agcaaaagcaggtcaaatatattcaatatggaaagaataaaagaactaagagatttgatgtcacagtctcgcactcgcgagatactgacaaaaacaactgtggaccatatggccataatcaagaaatatacatcaggaagacaggagaagaatcctgcccttaggatgaagtggatgatggcaatgaaatatccaattacggcagacaaaaggataatggagatgatcccggaaagaaatgagcaaggtcagaccctttggagcaagacaaatgatgctggatcagacagagtaccatttgcagcagccccgccggagcagagtaggatgcagttctcttctctaactgtgaatgtgaggggttccggaatgagaatagttgtgagaggcaattctcctgtgttcaactacaacaaggcaaccaagaggcttacggtgcttggaaaggatgcaggtgcattgatggaagaccccgatgagggaacagcaggagtggaatctgcggtattgaggggatttctgattctgggcaaagaaaacaaaagatatgggccagcattgagcatcaacgaattgagcaatcttgcgaaaggagagaaggctaatgtgttgatagggcaaggagacgttgtgttggtgatgaaacggaaacgggactctagcatacttactgacagtcagacagcgaccaaaaggattcggatggccatcaattaatgtcgaattgtttaaaaacgaccttgtttctact

SEQ ID NO: 16 - DNA, 416 bp, synthetic construct
  source 1..416: mol_type = other DNA
agcaaaagcaggtactgatccaaaatggaagactttgtgcgacagtgcttcaatccaatgatcgtcgagcttgcggaaaaggcaatgaaagaatatggggaagatccgaaaatcgaaacaaacaaattcgcatcaatatgtattcaacagtctatatgcatctccgcaactcgaggggttctcagctgaatcgagaaaactgctactcattgttcaggcgcttagggataacctggaacctggaaccttcgatcttgaagggctatatgaagcaatcgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 17 - DNA, 481 bp, synthetic construct
  source 1..481: mol_type = other DNA
agcaaaagcaggtactgatccaaaatggaagactttgtgcgacaatgcttcaatccaatgatcgtcgagcttgcggaaaaggcaatgaaagaatatggggaagatccgaaaatcgaaacaaacaaattcgcatcaatatgcacacacttagaagtctgcttcatgtactctgatttccactgcagaaccttactagcaaaatctgtattcaacagtctatatgcatctccgcaactcgaggggttctcagctgaatcgagaaaactgctactcattgttcaggcgcttagggataacctggaacctggaaccttcgatcttgaagggctatatgaagcaatcgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 18 - DNA, 506 bp, synthetic construct
  source 1..506: mol_type = other DNA
agcaaaagcaggtactgatccaaaatggaagactttgtgcgacaatgcttcaatccaatgatcgtcgagcttgcggaaaaggcaatgaaagaatatggggaagatccgaaaatcgaaacaaacaaattcgcatcaatatgcacacacttagaagtctgcttcatgtactctgattggagtggaggaaggttccatcgggaaggtgtgcagaaccttactagcaaaatctgtattcaacagtctatatgcatctccgcaactcgaggggttctcagctgaatcgagaaaactgctactcattgttcaggcgcttagggataacctggaacctggaaccttcgatcttgaagggctatatgaagcaatcgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 19 - DNA, 623 bp, synthetic construct
  source 1..623: mol_type = other DNA
agcaaaagcaggtactgatccaaaatggaagactttgtgcgacaatgcttcaatccaatgatcgtcgagcttgcggaaaaggcaatgaaagaatatggggaagatccgaaaatcgaaacaaacaaattcgcatcaatatgcacacacttagaagtctgcttcatgtactctgatttccacttcatcgacgaacgaggcgaatcaactattatagaatctggcgatccaaatgtgctgctgaaacatctttgaaaacaaatctgaaacatggccaattggagaatcacccaaaggagtggaggaaggttccatcgggaaggtgtgcagaaccttactagcaaaatctgtattcaacagtctatatgcatctccgcaactcgaggggttctcagctgaatcgagaaaactgctactcattgttcaggcgcttagggataacctggaacctggaaccttcgatcttgaagggctatatgaagcaatcgaggagtgcctgattaatgatccctgggttttgcttaatgcatcttggttcaactccttcctcacacatgcactaagatagttgtggcaatgctactatttgctatccatactgtccaaaaaagtaccttgtttctact

SEQ ID NO: 20 - DNA, 458 bp, synthetic construct
  source 1..458: mol_type = other DNA
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacaaggaatcgctccattctcaacaccagccaaagggggattcttgaggacgaacagatgtaccagaagtgctgcaacctattcgaaaagttcttccccagcagttcgtacaggaggccagttggaatttccagcatggtggaggccatggtgtctagggcccgaattgatgcacgaattgacttcgaatctggaaggattaagaaagaagagttcgctgagatcatgaagatctgttccaccattgaagagctcagacggcaaaaatagtgaatttagcttgtccttcatgaaaaaatgccttgtttctact

SEQ ID NO: 21 - DNA, 470 bp, synthetic construct
  source 1..470: mol_type = other DNA
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacacagtcaacagaacacataaatactcagaaaaaggaaagtggacaacgaattcttgaggacgaacagatgtaccagaagtgctgcaacctattcgaaaagttcttccccagcagttcgtacaggaggccagttggaatttccagcatggtggaggccatggtgtctagggcccgaattgatgcacgaattgacttcgaatctggaaggattaagaaagaagagttcgctgagatcatgaagatctgttccaccattgaagagctcagacggcaaaaatagtgaatttagcttgtccttcatgaaaaaatgccttgtttctact

SEQ ID NO: 22 - DNA, 477 bp, synthetic construct
  source 1..477: mol_type = other DNA
agcaaaagcaggcaaaccatttgaatggatgtcaatccgactttacttttcttgccagtgcaaaatgctataagtaccactttcccttatactggagaccctccatacagccatggaacaggaacaggatacaccatggacacagtcaacagaacacataaatactcagaaaaaggaaagtggacaacgaacacagagactggagcaccccaactcaatccaattaccagaagtgctgcaacctattcgaaaagttcttccccagcagttcgtacaggaggccagttggaatttccagcatggtggaggccatggtgtctagggcccgaattgatgcacgaattgacttcgaatctggaaggattaagaaagaagagttcgctgagatcatgaagatctgttccaccattgaagagctcagacggcaaaaatagtgaatttagcttgtccttcatgaaaaaatgccttgtttctact

SEQ ID NO: 23 - DNA, 494 bp, synthetic construct
  source 1..494: mol_type = other DNA
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacacagtcaacagaacacataaatactcagaaaaaggaaagtggacaacgaacacagagactggagcaccccaactttcttgaggacgaacagatgtaccagaagtgctgcaacctattcgaaaagttcttccccagcagttcgtacaggaggccagttggaatttccagcatggtggaggccatggtgtctagggcccgaattgatgcacgaattgacttcgaatctggaaggattaagaaagaagagttcgctgagatcatgaagatctgttccaccattgaagagctcagacggcaaaaatagtgaatttagcttgtccttcatgaaaaaatgccttgtttctact

SEQ ID NO: 24 - DNA, 690 bp, synthetic construct
  source 1..690: mol_type = other DNA
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacacagtcaacagaacacataaatactcagaaaaaggaaagtggacaacgaacacagagactggagcaccccaactcaatccaattgatggaccattacccgaggacaacgagccgagtgggtatgcacaaacggattgtgtattggaagcaatggctttccttgaagaatctcacccagggatctttgaaaactcgtgtctcgaaacgatggaaattgttcagcaaacaagagtggataaactgacccaaggccgccagacctatgactggacgttgaatagaaatcagccggctgctaccgcattggccaacactatagaggtattcagatcgaatggcctgacagccaatgaatcaggaaggttgatcgatttcctcaaggacgtgatggattcaatggataaggaagaaatggagattacaacacatttccagaggaagagttcgctgagatcatgaagatctgttccaccattgaagagctcagacggcaaaaatagtgaatttagcttgtccttcatgaaaaaatgccttgtttctact

SEQ ID NO: 25 - DNA, 808 bp, synthetic construct
  source 1..808: mol_type = other DNA
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacacagtcaacagaacacataaatactcagaaaaaggaaagtggacaacgaacacagagactggagcaccccaactcaatccaattgatggaccattacccgaggacaacgagccgagtgggtatgcacaaacggattgtgtattggaagcaatggctttccttgaagaatctcacccagggatctttgaaaactcgtgtctcgaaacgatggaaattgttcagcaaacaagagtggataaactgacccaaggccgccagacctatgactggacgttgaatagaaatcagccggctgctaccgcattggccaacactatagaggtattcagatcgaatggcctgacagccaatgaatcaggaaggttgatcgatttcctcaaggacgtgatggattcaatggataaggaagaaatggagattacaacacatttcgaaaagttcttccccagcagttcgtacaggaggccagttggaatttccagcatggtggaggccatggtgtctagggcccgaattgatgcacgaattgacttcgaatctggaaggattaagaaagaagagttcgctgagatcatgaagatctgttccaccattgaagagctcagacggcaaaaatagtgaatttagcttgtccttcatgaaaaaatgccttgtttctact

SEQ ID NO: 26 - DNA, 925 bp, synthetic construct
  source 1..925: mol_type = other DNA
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacacagtcaacagaacacataaatactcagaaaaaggaaagtggacaacgaacacagagactggagcaccccaactcaatccaattgatggaccattacccgaggacaacgagccgagtgggtatgcacaaacggattgtgtattggaagcaatggctttccttgaagaatctcacccagggatctttgaaaactcgtgtctcgaaacgatggaaattgttcagcaaacaagagtggataaactgacccaaggccgccagacctatgactggacgttgaatagaaatcagccggctgctaccgcattggccaacactatagaggtattcagatcgaatggcctgacagccaatgaatcaggaaggttgatcgatttcctcaaggacgtgatggattcaatggataaggaagaaatggagattacaacacatttccagaggaagaggagagtgagggacaacatgaccaagaaaatggtcacacagagaacaataggaaagaaaaaacaaagactgaacaaaaggagctacctaataagagcactgacattgaacacaatgacaaaggatgctgaaagaggcaagctgaaaaggagggcaatcgcaacacccgggatgcaaatcagaggattcgtgtattttgtagaagcactagcgaggagcatctgtgagaaacttgagttcgctgagatcatgaagatctgttccaccattgaagagctcagacggcaaaaatagtgaatttagcttgtccttcatgaaaaaatgccttgtttctact

SEQ ID NO: 27 - DNA, 952 bp, synthetic construct
  source 1..952: mol_type = other DNA
agcaaaagcaggcaaaccatttgaatggatgtcaatccgactttacttttcttgaaagtgccagtgcaaaatgctataagtaccactttcccttatactggagaccctccatacagccatggaacaggaacaggatacaccatggacacagtcaacagaacacataaatactcagaaaaaggaaagtggacaacgaacacagagactggagcaccccaactcaatccaattgatggaccattacccgaggacaacgagccgagtgggtatgcacaaacggattgtgtattggaagcaatggctttccttgaagaatctcacccagggatctttgaaaactcgtgtctcgaaacgatggaaattgttcagcaaacaagagtggataaactgacccaaggccgccagacctatgactggacgttgaatagaaatcagccggctgctaccgcattggccaacactatagaggtattcagatcgaatggcctgacagccaatgaatcaggaaggttgatcgatttcctcaaggacgtgatggattcaatggataaggaagaaatggagattacaacacatttccagaggaagaggagagtgagggacaacatgaccaagaaaatggtcacacagagaacaataggaaagaaaaaacaaagactgaacaaaaggagctacctaataagagcactgacattgaacacaatgacaaaggatgctgaaagaggcaagctgaaaaggagggcaatcgcaacacccgggatgcaaatcagaggattcgtgtattttgtagaagcactagcgaggagcatctgtgagaaacttgagcaatctggaaggattaagaaagaagagttcgctgagatcatgaagatctgttccaccattgaagagctcagacggcaaaaatagtgaatttagcttgtccttcatgaaaaaatgccttgtttctact

SEQ ID NO: 28 - DNA, 18 bp, synthetic construct
  source 1..18: mol_type = other DNA
atggaaagaataaaagaa

SEQ ID NO: 29 - DNA, 21 bp, synthetic construct
  source 1..21: mol_type = other DNA
atcagaatggctattaactaa

SEQ ID NO: 30 - DNA, 18 bp, synthetic construct
  source 1..18: mol_type = other DNA
atggagcggatcaaggag

SEQ ID NO: 31 - DNA, 21 bp, synthetic construct
  source 1..21: mol_type = other DNA
attcggatggccatcaattaa

SEQ ID NO: 32 - DNA, 12 bp, synthetic construct
  source 1..12: mol_type = other DNA
agcaaaagcagg

(19) United States
(12) Patent Application Publication
(10) Pub. No.: US 2025/0313813 A1
(43) Pub. Date: Oct. 9, 2025

(54) RATIONALLY DESIGNED SINGLE-ROUND INFECTIOUS VIRUS AND METHODS OF USE THEREOF

(71) Applicants: Versitech Limited, Hong Kong (CN); Centre For Virology, Vaccinology and Therapeutics Limited, Hong Kong (CN)

(72) Inventors: Kin Hang Kok, Hong Kong (CN); Chun Kit Yuen, Hong Kong (CN); Kwok Yung Yuen, Hong Kong (CN); Wan Man Wong, Hong Kong (CN)

(21) Appl. No.: 18/728,849

(22) PCT Filed: Jan. 19, 2023
(86) PCT No.: PCT/CN2023/073098

§ 371 Date: Jul. 12, 2024

Related U.S. Application Data
(60) Provisional application No. 63/300,864, filed on Jan. 19, 2022.

(51) Int. Cl. C12N 7/00; A61K 39/00; A61K 39/145; A61P 37/04

(52) U.S. Cl. CPC C12N 7/00; A61K 39/145; A61P 37/04; A61K 2039/54; C12N 2760/16121; C12N 2760/16134; C12N 2760/16151

(57) ABSTRACT

Engineered, replication-deficient influenza viruses that are infectious and stimulate broadly-cross protective immunity against multiple different influenza strains have been developed. Compositions and methods for providing protective immune responses against influenza are provided. The methods deliver compositions of intact, replication-deficient influenza viruses by intradermal administration in an amount effective to elicit or stimulate a cross-protective immune response to influenza viruses in the recipient following a single administration. Because the engineered influenza viruses are non-replicating, they are safe and effective in immunocompromised subjects. Methods for delivering co-stimulatory molecules, growth factors, adjuvants and/or cytokines together with the non-replicating influenza viruses are also provided.

### CROSS-REFERENCE TO RELATED APPLICATIONS

This application is a National Phase application under 35 U.S.C. 371 of PCT/CN2023/073098, filed Jan. 19, 2023, which claims the benefit of and priority to U.S. Application No. 63/300,864 filed Jan. 19, 2022, the contents of which are incorporated herein by reference in their entirety.

### REFERENCE TO SEQUENCE LISTING

The Sequence Listing submitted as an XML file named “UHK_01161_371_ST26.xml,” created on Jan. 9, 2025, and having a size of 53,331 bytes is hereby incorporated by reference pursuant to 37 C.F.R. § 1.834(c)(1).

### FIELD OF THE INVENTION

The invention is generally in the field of vaccines and specifically in the area of using recombinant, non-replicating influenza viruses for inducing broadly cross-protective immunity to influenza viruses of different lineages.

### BACKGROUND OF THE INVENTION

Influenza is one of the leading global health concerns. Every year, seasonal influenza viruses infect the human population, causing millions of severe infections and several hundred thousand deaths. Each year in the United States, an estimated 36,000 deaths and millions of hospitalizations are due to influenza-related illness. Globally, influenza is associated with an estimated 250,000-500,000 deaths annually. In the case of influenza pandemics, viruses resulting from antigenic shift enter the human population to cause even greater mortality and morbidity, as witnessed in the 1918 Spanish flu and the 2011 swine flu pandemics.

The primary defense against influenza is mass vaccination, and tremendous effort has been spent on influenza vaccine development to control the disease spreading and to alleviate the associated economic loss. Current vaccine strategies against influenza viruses aim to induce immune protection based on a robust antibody response in the host. Currently, two major types of vaccines, namely inactivated/split influenza vaccine and live-attenuated influenza vaccine (LAIV), are commercially available and widely used. Inactivated vaccine mainly elicits humoral response by inducing production of serum neutralizing antibodies, the level of which is the major determinant of vaccine efficacy (Potter, Br Med Bull 35, 69-75 (1979); Hirota, et al., Vaccine 15, 962-967 (1997)). The merit of inactivated vaccine is that it is composed of destroyed virions and so causes little safety concern. LAIV, on the contrary, contains live viruses but temperature sensitive. The vaccination mimics a natural infection and is conceived to be superior to vaccination with destroyed viruses. Interestingly, LAIV was reported to trigger weaker humoral response compared with inactivated vaccine but could induce mucosal and cell-mediated immunity (He, et al. J Virol 80, 11756-11766 (2006)). However, LAIV is not recommended for people with compromised immunity due to possible viral replication.

Besides testing different vaccine regimens, various modifications of the existing vaccines have also been sought to improve vaccine efficacy. Based on the inactivated vaccines already in use, strategies including addition of adjuvants (e.g., FLUAD) and administration of higher vaccine dose (e.g., Fluzone High-Dose) have been adopted to either promote innate immune activation or increase neutralizing antibody production.

The route of administration has also been examined for its contribution to vaccination effectiveness. The current trivalent or quadrivalent inactivated vaccines are routinely administered through intramuscular route, whereas LAIV is delivered intranasally. Intriguingly, intradermal immunization has recently emerged with promising dose-sparing effect (Hung, et al., Hum Vaccin Immunother 14, 565-570 (2018)). Compared with intramuscular vaccination, intradermal administration of the same influenza vaccine with five-time spared dose showed similar immunogenicity (Hung, et al., Vaccine 30, 2707-2708 (2012); Kenney, et al., N Engl J Med 351, 2295-2301 (2004)). Furthermore, pre-clinical evaluation in mice and human clinical trials of intradermal trivalent vaccine clearly demonstrated that pretreatment of imiquimod, an activator of innate immune response, markedly improved the vaccine's immunogenicity as evidenced by the high seroconversion rate and better protection in phase 2b/3 clinical trial (Zhang, et al., Clin Vaccine Immunol 21, 570-579 (2014); Hung, et al., Clin Infect Dis 59, 1246-1255 (2014); Hung, et al., Lancet Infect Dis 16, 209-218 (2016)). However, none of the currently available vaccine reagents can be effectively administered intradermally. None of the currently available vaccine reagents and regimens can elicit heterologous protection against multiple different influenza viruses from different lineages.

One potential avenue for development of effective influenza vaccines is through the production and development of defective interfering (DI) virus. Influenza virus is a negative sense RNA virus featured with a segmented genome (Bouvier and Palese, Vaccine 26 (2008): D49-D53). Among the eight segments of the viral genome, defective genomes are mainly found in segments one to three which encode the polymerase subunits polymerase basic 2 (PB2), polymerase basic 1 (PB1) and polymerase acidic (PA) (Fields, and Winter, Cell 28.2 (1982): 303-313; Alnaji, et al., Journal of virology 93.11 (2019): e00354-19). The low fidelity of influenza RNA dependent RNA polymerase (RdRp) contributes to the error-prone replication of the influenza viral genome, which often occurs in the form of bulk internal deletions of the polymerase segments (Yang, et al., Frontiers in Microbiology (2019): 1852.). The defective viral genomes can be readily packaged into progeny virions, resulting in the formation of DI virus. DI virus can compete with standard genomes for replication, hence interfering with the life cycle of standard virus (Vignuzzi, and López, Nature microbiology 4.7 (2019): 1075-1087). DI virus is also a potent inducer of type I and III interferons (IFNs) as well as other pro-inflammatory cytokines such as interleukin (IL)-6 and IL-1β (Sun, et al. PLOS pathogens 11.9 (2015): e1005122). To date, there has been immense effort in exploring the antiviral and vaccine potential of DI virus, yet its translation into a clinical setting remains far-fetched (Dimmock, et al., Journal of Virology 82.17 (2008): 8570-8578; and Dimmock, et al., Antiviral research 96.3 (2012): 376-385). Different studies have attempted to evaluate the application potential of DI virus. The antiviral activities of DI244, a PB2 DI species derived from H1N1 (PR8), have been extensively studied (Dimmock, et al., Journal of Virology 82.17 (2008): 8570-8578). It has been shown that cloned DI virus bearing the DI244 genome can be generated by reverse genetics. The DI virus are then passaged in eggs alongside infectious helper virus, which are then subjected to ultraviolet irradiation to eliminate any standard virus activities remaining in the virus mixture. Prophylactic and therapeutic administration of DI244 protects mice and ferrets from lethal infection of pandemic 2009 influenza virus (Dimmock, et al., PLOS One 7.12 (2012): e49394). Complete protection can be observed in prophylactic administration and therapeutic administration 1 day after virus challenge, while treatment at 2 days after infection confers partial protection. The prophylactic protective efficacy is comparable to oseltamivir, the major therapeutic agent against influenza (Dimmock, et al., Antiviral research 96.3 (2012): 376-385). DI244 has also been shown to confer heterologous protection against paramyxoviridae virus and influenza B virus, which is likely mediated by the activation of innate immune response (Easton, et al., Vaccine 29.15 (2011): 2777-2784; Scott, et al., Journal of general virology 92.9 (2011): 2122-2132). The multifaceted approach of DI virus in viral inhibition makes it a fine candidate of broad-spectrum prophylactic agent against respiratory virus. However, there are many factors hindering the clinical application of DI virus. The presence of standard virus during DI virus production poses concerns over its safety and purity and the presence of full-length polymerase genome also poses a risk of genetic reassortment upon therapeutic treatment against standard virus infection in vivo.

Therefore, it is an object of the invention to provide vaccine reagents capable of inducing broad-acting immunity against multiple different influenza viruses.

It is also an object of the invention to provide methods for the efficient production of pure, defective interfering (DI) virus with high yield.

It is also an object of the invention to provide methods for providing long-term and broadly protective immunity against current and emerging influenza viruses.

### SUMMARY OF INVENTION

It has been established that intradermal immunization with live, modified, replication-deficient influenza virus generates a more-broadly-protective anti-influenza immune response and stronger protection of the immunized host at a much lower dose compared to conventional inactivated “split” influenza vaccines delivered via other injection routes.

Modified, intact, replication-deficient influenza viruses that infect normal human cells are provided. The virus includes an influenza virus genome that includes one or more mutations in one or more genes selected from the group including viral RNA polymerase PB1, viral RNA polymerase PB2, viral RNA polymerase PA, and nucleoprotein (NP), whereby the one or more mutations prevents or reduces replication of the virus in normal human cells by at least 90% as compared to the same virus in the absence of the mutation.

In some forms, the influenza virus genome includes one or more mutations in the viral RNA polymerase PB2 gene that prevents or reduces replication of the virus in normal human cells. In particular forms, the virus is 100% non-replicating in normal mammalian cells.

In an exemplary form, the influenza virus genome includes a viral RNA polymerase PB2 gene having the nucleic acid sequence of SEQ ID NO:1, or a nucleic acid sequence having at least 75% identity to SEQ ID NO:1. In other forms, the influenza virus genome includes a viral RNA polymerase PB2 gene having the nucleic acid sequence of any one of SEQ ID NOs: 9-15, or a nucleic acid sequence having at least 75% identity to any one of SEQ ID NOs: 9-15. In some forms, the influenza virus genome includes one or more mutations in the viral RNA polymerase PA gene that prevents or reduces replication of the virus in normal human cells. For example, in some forms, the influenza virus genome includes a viral RNA polymerase PA gene having the nucleic acid sequence of any one of SEQ ID NOs: 16-19, or a nucleic acid sequence having at least 75% identity to any one of SEQ ID NOs: 16-19. In some forms, the influenza virus genome includes one or more mutations in the viral RNA polymerase PB1 gene that prevents or reduces replication of the virus in normal human cells. For example, in some forms, the influenza virus genome includes a viral RNA polymerase PB1 gene having the nucleic acid sequence of any one of SEQ ID NOs: 20-27, or a nucleic acid sequence having at least 75% identity to any one of SEQ ID NOs: 20-27. In some forms, the influenza virus genome includes eight genomic segments, whereby between one and seven of the genomic segments are derived from a first influenza virus, and whereby between one and seven of the genomic segments are derived from a second influenza virus, and whereby the one or more mutations that prevent or reduce viral replication are present in the genomic segments derived from the second virus. For example, in some forms, the genome includes between five and seven genomic segments of the first influenza virus, wherein the first virus is a replication-competent influenza A virus selected from (i) the group including H1, H2, H3, H5, H6, H7, H9, and H10 subtypes; and (ii) the group including N1, N2, N6, N7, N8 and N9 subtypes. In some forms, the second virus is an influenza virus selected from the (i) group including H1, H2, H3, H5, H6, H7, H9, and H10 subtype, and (ii) the group including N1, N2, N6, N7, N8 and N9 subtypes.

An intact, replication-deficient virus that infects normal human cells, including an influenza virus genome, having eight genomic segments, whereby between one and seven of the segments are derived from a first influenza virus, and between one and seven of the segments are derived from a second influenza virus, wherein the genome includes one segment including a viral RNA polymerase PB1 gene and one segment including a viral RNA polymerase PB2 gene, wherein the influenza virus genome includes one or more mutations in the viral RNA polymerase PB1 gene and one or more mutations in the viral RNA polymerase PB2 gene, wherein the one or more mutations prevent or reduce replication of the virus in normal human cells by at least 90% as compared to the same virus in the absence of the mutation is also described. In some forms, the viral RNA polymerase PB1 gene has the nucleic acid sequence of any one of SEQ ID NOs: 20-27, or a nucleic acid sequence having at least 75% identity to any one of SEQ ID NOs: 20-27. In some forms, the viral RNA polymerase PB2 gene has the nucleic acid sequence of any one of SEQ ID NOs: 1, or 9-15 or a nucleic acid sequence having at least 75% identity to any one of SEQ ID NOs: 1, or 9-15. In some forms, the first influenza virus is a replication-competent influenza A virus selected from (i) the group including H1, H2, H3, H5, H6, H7, H9, and H10 subtypes; and (ii) the group including N1, N2, N6, N7, N8 and N9 subtypes. In some forms, the second virus is an influenza virus selected from (i) the group including H1, H2, H3, H5, H6, H7, H9, and H10 subtypes; and (ii) the group including N1, N2, N6, N7, N8 and N9 subtypes. In some forms, the influenza virus genome further includes one or more mutations in the one or more genes selected from viral RNA polymerase PA, and nucleoprotein (NP). For example, in some forms, the influenza virus genome includes a viral RNA polymerase PA gene having the nucleic acid sequence of any one of SEQ ID NOs: 16-19, or a nucleic acid sequence having at least 75% identity to any one of SEQ ID NOs: 16-19. In some forms, the genome includes between five and seven genomic segments of the first influenza virus. In some forms, the first and second viruses are of different subtypes. In other forms, the first and second viruses are different strains of the same subtype. In particular forms, the virus is 100% non-replicating in normal mammalian cells. In exemplary forms, the first influenza virus is selected from the group including N1N1, H3N1, H5N1, H7N9 and H2N2 subtypes. For example, in some forms, the first or second influenza virus is A/WSN/1933 (H1N1), or A/PR8/34 (H1N1), or A/HK/415742/2009 (H1N1), or (A/HK/4801/2014) (H3N2). In some forms, the influenza virus genome includes between one and three genomic segments derived from the second influenza virus, and the second virus is selected from the group including H5N1 and H7N9 subtypes. In some forms, the genome includes a mutated PB1, PB2 and/or PA gene derived from an H7N9 virus. In some forms, the virus includes one or more exogenous genes derived from a defective-interfering (DI) particle.

Exemplary intact, non-replicating viruses including an influenza virus genome wherein the virus infects normal human cells are described. In a first example, the influenza virus genome includes (i) genomic segments 4-8 of the A/WSN/1933 (H1N1) genome having a nucleic acid sequence of SEQ ID NOS: 4-8; and (ii) genomic segment 1 including a mutated PB2 gene having a nucleic acid sequence of any one of SEQ ID NOs: 1, or 9-15, or a nucleic acid sequence having at least 75% identity to any one of SEQ ID NO:1, or 9-15; and (iii) genomic segments 2 and 3 including the PB1 and PA genes of an H7N9 virus having a nucleic acid sequence of SEQ ID NOS: 2-3; and wherein the virus is completely non-replicating in normal human cells.

In a second example, the influenza virus genome includes (i) genomic segments 4 and 6 of the A/HK/4801/2014 (H3N2) genome; and (ii) genomic segment 1 including a mutated PB2 gene having a nucleic acid sequence of SEQ ID NO:11, 13 or 15, or a nucleic acid sequence having at least 75% identity to SEQ ID NO 11, 13 or 15, and (iii) genomic segments 2 and 3 including the PB1 and PA genes of an H7N9 virus having a nucleic acid sequence of SEQ ID NOS: 2-3; and wherein the virus is completely non-replicating in normal human cells. In some forms, the intact, non-replicating virus of includes genomic segments 7 and 8 from A/PR8/34 (H1N1).

In a third example, the influenza virus genome includes (i) genomic segments 4 and 6-8 of the A/HK/415742/2009 (H1N1) genome; and (ii) genomic segment 1 including a mutated PB2 gene having a nucleic acid sequence of SEQ ID NO:13, or a nucleic acid sequence having at least 75% identity to SEQ ID NO:13, and (iii) genomic segments 2 and 3 including the PB1 and PA genes of an H7N9 virus having a nucleic acid sequence of SEQ ID NOS: 2-3; and wherein the virus is completely non-replicating in normal human cells.

In a fourth example, the influenza virus genome includes (i) genomic segments 4 and 6-8 of the A/HK/415742/2009 (H1N1) genome; and (ii) genomic segment 2 including a mutated PB1 gene having a nucleic acid sequence of SEQ ID NO:26 or 27, or a nucleic acid sequence having at least 75% identity to SEQ ID NO:26 or 27, and (iii) genomic segments 1 and 3 including the PB2 and PA genes of an H7N9 virus; and wherein the virus is completely non-replicating in normal human cells.

In a fifth example, the influenza virus genome includes (i) genomic segments 4 and 6-8 of the A/PR8/34 (H1N1) genome; and (ii) genomic segment 2 including a mutated PB1 gene having a nucleic acid sequence of SEQ ID NO:22, or a nucleic acid sequence having at least 75% identity to SEQ ID NO:22, (iii) genomic segment 1 including a mutated PB2 gene having a nucleic acid sequence of SEQ ID NO:14, or a nucleic acid sequence having at least 75% identity to SEQ ID NO:14, and (iv) genomic segment 3 including the PA gene of an H7N9 virus having a nucleic acid sequence of SEQ ID NO:3; and wherein the virus is completely non-replicating in normal human cells.

In a sixth example, the influenza virus genome includes (i) genomic segments 4 and 6-8 of the A/PR8/34 (H1N1) genome; and (ii) genomic segment 1 including a mutated PB1 gene having a nucleic acid sequence of SEQ ID NO:1, or a nucleic acid sequence having at least 75% identity to SEQ ID NO:1, and (iii) genomic segments 2 and 3 including a PB2 and PA genes of an H7N9 virus having a nucleic acid sequence of SEQ ID NO:2 and 3, respectively; and wherein the virus is completely non-replicating in normal human cells.

In a seventh example, the influenza virus genome includes (i) genomic segments 4 and 6-8 of the A/PR8/34 (H1N1) genome; and (ii) genomic segment 2 including a mutated PB1 gene having a nucleic acid sequence of SEQ ID NO:22, or a nucleic acid sequence having at least 75% identity to SEQ ID NO:22, (iii) genomic segments 1 and 3 including the PA gene of an H7N9 virus; and wherein the virus is completely non-replicating in normal human cells.

In an eighth example, the influenza virus genome includes (i) genomic segments 4-8 of the A/WSN/1933 (H1N1) genome having a nucleic acid sequence of SEQ ID NOS: 4-8; and (ii) genomic segment 1 including a mutated PB2 gene having a nucleic acid sequence of SEQ ID NO:13, or a nucleic acid sequence having at least 75% identity to any one of SEQ ID NO:13; (iii) genomic segment 2 including a mutated PB1 gene having a nucleic acid sequence of SEQ ID NO:22, or a nucleic acid sequence having at least 75% identity to any one of SEQ ID NO:22; and (iv) genomic segment 3 including the PA gene of an H7N9 virus having a nucleic acid sequence of SEQ ID NO:3; and wherein the virus is completely non-replicating in normal human cells.

In a ninth example, the influenza virus genome includes (i) genomic segments 4-8 of the A/WSN/1933 (H1N1) genome having a nucleic acid sequence of SEQ ID NOS: 4-8; and (ii) genomic segment 2 including a mutated PB1 gene having a nucleic acid sequence of SEQ ID NO:22, or a nucleic acid sequence having at least 75% identity to any one of SEQ ID NO: 22; and (iii) genomic segments 1 and 3 including the PA gene of an H7N9 virus; and wherein the virus is completely non-replicating in normal human cells. Typically, the virus morphology includes a spherical or elliptical virion having a diameter of between about 80 nm and about 120 nm, inclusive.

Vaccine compositions that provide immunity to influenza viruses in a subject are also provided. Typically, the vaccines include (i) an intact, replication-deficient virus of, as described above, and (ii) a pharmaceutically acceptable excipient suitable for intradermal administration, whereby the composition is in an amount effective to induce a protective immune response to one or more influenza viruses in the subject following intradermal administration to the subject. In some forms, the composition is in an amount effective to induce a protective immune response to one or more of an H1/N1, H3/N2 or H5/N1 influenza virus. In some forms, the composition further including one or more additional agents selected from group including co-stimulatory molecules, growth factors, adjuvants, and cytokines. Exemplary additional agents include IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Flt3-ligand, and CCR4.

Methods for inducing or stimulating a protective immune response to an influenza virus in a subject, including administering to epidermal tissues of the subject the vaccine composition as described above, in an amount effective to induce or stimulate the immune response in the subject, are also provided. In some forms, the methods administer the vaccine composition to the subject by intradermal injection. In some forms, the methods further include administering to the subject one or more additional agents selected from the group including an anti-infective agent, a co-stimulatory molecule, a growth factor, an adjuvant and/or cytokine, wherein the one or more additional agents are administered before, at the same time, or after administering the vaccine composition. Exemplary additional agents include IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Flt3-ligand, and CCR4. In some forms, the methods include repeating the step of administering the vaccine composition to the subject, for example, at a time of one, two, three, four, five, six, seven, eight, nine, or ten days or weeks after the first administration. In some forms, the methods provide protective immunity to two or more different strains of influenza viruses. For example, in some forms the method provides protective immunity to one or more H1N1 influenza viruses and one or more H3N2 influenza viruses, and/or one or more H5N1 influenza viruses and/or one or more H7N9 influenza viruses.

Kits including the vaccine compositions as described above and optionally one or more devices for intradermal administration of the composition to a subject are also provided. Also provided is a dosage unit for immunization by intradermal administration including an effective amount of the vaccine composition ss described above for inducing or stimulating a protective immune response to an influenza virus in a subject.

Methods of making the described intact, replication-deficient virus including an influenza virus genome, having one or more mutations in one or more genes selected from viral RNA polymerase PB1, viral RNA polymerase PB2, viral RNA polymerase PA, and nucleoprotein (NP) are also described. Typically, the methods include one or more steps of (a) introducing into a first cell genes encoding 4-7 segments of a wild-type virus; and (b) introducing into a second cell gene(s) encoding a mutant PB1, PB2, PA, and/or NP, (c) co-culturing of the first and second cells; and (d) isolating the intact, replication-deficient virus. In some forms, the first and/or second cell is selected from the group including a MDCK cell and a 293FT cell. In exemplary forms, the mutant PB1, PB2, PA, and/or NP gene(s) is introduced into the cell by lentivirus transduction. Typically, the mutant PB1, PB2, PA, and/or NP gene(s) is within an expression vector which is driven by an RNA polymerase I promoter. In some forms, isolating in step (d) includes purification by filtration, for example, by passing through one or more 0.45 μm filter. In some forms, isolating in step (d) includes purification by ultracentrifugation, such as sucrose-cushioned ultracentrifugation, for example, at 28,000 rpm for 4 hours. An intact, replication-deficient virus and a cell expressing an intact, replication-deficient virus produced according to the methods of making is also described.

### BRIEF DESCRIPTION OF DRAWINGS

FIG. 1 is a schematic showing the viral genomes of parental and recombinant viruses. Solid lines represent viral segments of H1N1, and light and dark dotted lines represent viral segments of H7N9 and H5N1 respectively.

FIGS. 2A-2B show the structure of an engineered, non-functional PB2 gene. FIG. 2A shows the nucleotide sequence of the sense strand of the most abundantly expressed defective-interfering (DI) genome (SEQ ID NO:1), which was derived from the A/H7N9 PB2 segment (named as PB2-DI). The dotted line indicates the breakpoint. FIG. 2B is a schematic showing silent mutations of PB2 transgene. The nucleic acid sequences shown include ATGGAAAGAATAAAAGAA (SEQ ID NO:28); ATCAGAATGGCTATTAACTAA (SEQ ID NO:29); ATGGAGCGGATCAAGGAG (SEQ ID NO:30); and ATTCGGATGGCCATCAATTAA (SEQ ID NO:31). Nucleotides underlined represent the synonymous mutations introduced at 5′ and 3′ end of the PB2 transgene.

FIGS. 3A-3F are a schematic and data showing the steps of IDIV synthesis. FIG. 3A is a schematic of a reverse-genetics expression system using H7N9 and H1N1-WSN co-transfected to MDCK-2P and 293FT-2P to generate a recombinant PB2-DI virus. Expression plasmids for WSN segments (HA, NP, NA, M and NS; solid-lines) and H7N9 segments (PB2-DI, PB1 and PA; dotted-lines) were co-transfected into the co-culture of 293-PB2 and MDCK-PB2 cells that stably express PB2 protein. Viral supernatant was collected for plaque-purification. Plaque-purified IDIV was propagated in MDCK-PB2 cells and further purified by sucrose cushion ultracentrifugation. FIGS. 3B-3D are images of plaque assays showing IDIV can only propagated in MDCK-PB2 cells. Absence of replicative virus in the IDIV was confirmed by plaque assay on parental MDCK cells (FIG. 3B) and titer of IDIV before and after ultracentrifugation was determined by plaque assay on MDCK-PB2 stable cells, before (FIG. 3C) and after ultracentrifugation (FIG. 13D) cells, respectively. FIGS. 3E-3F are images of a gel confirming the presence of stable PB2-DI (FIG. 3E) and NP (FIG. 3F) in each of 5 passages (P1-P5) respectively, showing a negative control (−ve) and molecular size marker for bands in lanes corresponding to P1, P2, P3, P4, and P5, respectively.

FIGS. 4A-4J are graphs showing concentrations (in pg/mL) of cytokines produced by human peripheral blood-derived macrophages (PBDM) and monocyte-derived dendritic cells (MoDC) after IDIV infection. PBDM and MoDC derived from four donors (Donor A (▴); Donor B (▪); Donor C (▾); and Donor D (●)) were infected with IDIV and supernatant of infected cells were harvested at 0-, 3-, 6- and 12-hours post-infection (hpi). Concentrations (in pg/mL) of cytokines in the supernatant produced by PBDM were quantitated including IP10 (FIG. 4A, 0-20,000 μg/mL), IFNβ (FIG. 4B, 0-500 μg/mL), IFN λ 1 (FIG. 4C, 0-600 μg/mL), IFNα2 (FIG. 4D, 0-500 μg/mL), and IFN λ 2/3 (FIG. 4E, 0-500 μg/mL). Concentrations (in pg/mL) of cytokines in the supernatant produced by MoDC were quantitated including IP10 (FIG. 4F, 0-1,500 μg/mL), IFNβ (FIG. 4G, 0-800 μg/mL), IFN λ 1 (FIG. 4H, 0-1,500 μg/mL), IFNα2 (FIG. 4I, 0-500 μg/mL), and IFN λ 2/3 (FIG. 4J, 0-500 μg/mL).

FIGS. 5A-5H are graphs showing serum neutralizing antibody response in immunized mice. FIGS. 5A and 5B show hemagglutination inhibition (HAI) titer (FIG. 5A, 0-640) and microneutralization (MN) titer (FIG. 5B, 0-640) at day 28 post-vaccination in sera of BALB/c mice intra-dermally vaccinated with IDIV at increasing dose of 0, 2×10², 2×10⁴, 2×10⁶ or 2×10⁸ PFU/mouse (n=3); FIGS. 5C and 5D show HAI titer (FIG. 5C, 0-640) and MN titer (FIG. 5D, 0-640) in sera of BALB/c mice intra-dermally vaccinated with 2×10⁸ PFU IDIV (n=5) at day 7, 14, and 28 post-vaccination. FIGS. 5E and 5F show HAI titer (FIG. 5E, 0-640) and MN titer (FIG. 5F, 0-640) in sera of quadrivalent vaccine-immunized mice at day 7, 14, and 28 post-vaccination (n=5). FIGS. 5G and 5H show % NP positive cells (0-120%) in serum dilutions including 1:160, 1:320, 1:640, and 1:1,280 in IDIV vaccine-immunized mice (FIG. 5G), or quadrivalent vaccine-immunized mice (FIG. 5H) versus PBS-immunized mice using Fluorescent Focus Microneutralization (FFMN) assay after 28 days of immunization. Representative figure from one out of five mouse serum samples was shown.

FIGS. 6A-6O are dot plots showing immunoglobulin isotypes in sera of vaccinated mice. Concentrations (in μg/mL) of immunoglobulin isotypes including IgG2a (FIGS. 6A-6C), IgA (FIGS. 6D-6F), IgG1 (FIGS. 6G-61), IgM (FIGS. 6J-6L) and IgG2b (FIGS. 6M-60) antibodies in the sera of mock-vaccinated (PBS), IDIV-vaccinated (IDIV), and QIV-vaccinated (Quadrivalent Influenza Vaccine) mice were quantitated using bead-based immunoassay at day 7, 14, and 28 post-vaccination. Statistical analysis between PBS- and IDIV-vaccinated groups was performed using unpaired t-test. *, p<0.05; n.s., not significant.

FIGS. 7A-7I are graphs showing schematic timeline of immunization and infection after IDIV, body weight changes, and survival data of vaccination against homologous virus challenges. FIGS. 7A-7C show mice intra-dermally vaccinated with PBS or IDIV at 28-day prior to viral challenge with A/H1N1 (WSN) virus (FIG. 7A), and their body weight change (FIG. 7B, 70%-110%) and survival (FIG. 7C, 0%-100%) monitored over 14 days post infection. FIGS. 7D-7F show mice intra-dermally vaccinated with PBS or IDIV at 14-day prior to viral challenge with A/H1N1 (WSN) virus (FIG. 7D), and their body weight change (FIG. 7E, 70%-110%) and survival (FIG. 7F, 0%-100%) monitored over 14 days post infection. FIGS. 7G-7I show mice intra-dermally vaccinated with PBS or IDIV at 7-day prior to viral challenge with A/H1N1 (WSN) virus (FIG. 7G), and their body weight change (FIG. 7H, 70%-110%) and survival (FIG. 7I, 0%-100%) monitored over 14 days post infection. Survival of mice was recorded using 20% weight loss as cut-off (lower panels). N=3 for all groups in 28-day immunization. N=5 and 6 for PBS and IDIV groups respectively for 14-day immunization. N=6 for both PBS and IDIV groups for 7-day immunization.

FIGS. 8A-8R are graphs showing schematic timeline of immunization and infection after IDIV, body weight changes, and survival data of vaccination against heterologous virus challenges. FIGS. 8A-8C show mice intra-dermally vaccinated with PBS or IDIV at 28-day prior to lethal viral challenge with heterosubtypic H1N1/pdm09 virus (FIG. 8A), and their body weight change (FIG. 8B, 60%-110%) and survival (FIG. 8C, 0%-100%) monitored over 14 days post infection. FIGS. 8D-8F show mice intra-dermally vaccinated with PBS or IDIV at 28-day prior to lethal viral challenge with heterologous H5N1/VN04 virus (FIG. 8D), and their body weight change (FIG. 8E, 70%-110%) and survival (FIG. 8F, 0%-100%) monitored over 14 days post infection. FIGS. 8G-8I show mice intra-dermally vaccinated with PBS or IDIV-2 vaccine that contains H1N1/pdm09 HA and NA at 28-day prior to lethal viral challenge with homologous H1N1/pdm09 virus (FIG. 8G), and their body weight change (FIG. 8H, 70%-110%) and survival (FIG. 8I, 0%-100%) monitored over 14 days post infection. FIGS. 8J-8L show mice intra-dermally vaccinated with PBS or IDIV-2 vaccine that contains H1N1/pdm09 HA and NA at 28-day prior to lethal viral challenge with heterosubtypic H1N1/PR8 virus (FIG. 8J), and their body weight change (FIG. 8K, 70%-110%) and survival (FIG. 8L, 0%-100%) monitored over 14 days post infection. FIGS. 8M-80 show mice intra-dermally vaccinated with PBS or IDIV-2 vaccine that contains H1N1/pdm09 HA and NA at 28-day prior to lethal viral challenge with heterologous H5N1/VN04 virus (FIG. 8M), and their body weight change (FIG. 8N, 70%-110%) and survival (FIG. 8O, 0%-100%) monitored over 14 days post infection. FIGS. 8P-8R show mice intra-dermally vaccinated with PBS or IDIV-2 vaccine that contains H1N1/pdm09 HA and NA at 28-day prior to lethal viral challenge with heterologous H7N9/AH1 virus (FIG. 8P), and their body weight change (FIG. 8Q, 70%-110%) and survival (FIG. 8R, 0%-100%) monitored over 14 days post infection.

FIGS. 9A-9L are graphs showing cytokine/chemokine concentrations (in pg/mL) in sera of mice at 18 hours post mock-vaccination (PBS), IDIV-vaccination (IDIV), or QIV-vaccination (QIV), including CXCL10 (FIG. 9A, 0-10,000 μg/mL), CCL2 (FIG. 9B, 0-1,000 μg/mL), IFNγ (FIG. 9C, 0-1,000 μg/mL), IFNα (FIG. 9D, 0-2,000 μg/mL), TNFα (FIG. 9E, 0-500 μg/mL), IL-6 (FIG. 9F, 0-500 μg/mL), CXCL1 (FIG. 9G, 0-500 μg/mL), CCL5 (FIG. 9H, 0-500 μg/mL), IL1β (FIG. 9I, 0-500 μg/mL), IL-10 (FIG. 9J, 0-500 μg/mL), IFNβ (FIG. 9K, 0-500 μg/mL), and GM-CSF (FIG. 9L, 0-500 μg/mL). Statistical analysis between PBS- and IDIV-vaccinated groups was performed using unpaired t-test. ***, p<0.001; *, p<0.05; n.s., not significant; N.D., not detected.

FIGS. 10A-10B are graphs showing HAI assay showing HAI titer (0-640) (FIG. 10A) and MN titer (0-640) (FIG. 10B) in sera of A129 mice vaccinated with 2×10⁸ PFU IDIV intra-dermally at day 7 (●), 14 (▪), and 28 (▴) post-vaccination. FIGS. 10C-10D are graphs showing HAI titer (FIG. 10C) and MN titer (FIG. 10D) for a quadrivalent vaccine at day 7 (●), 14 (▪), and 28 (▴) post-vaccination.

FIGS. 11A-11C show the generation of H1N1/WSN/PB1-477. FIG. 11A is a schematic of a reverse-genetics expression system using H1N1 and WSN co-transfected to MDCK-2P and 293FT-2P to generate a recombinant PB1-DI virus. FIG. 11B is an image of a gel confirming the incorporation of the PB1-DI477 into the viral genome, showing molecular size marker for bands in lanes corresponding to PB2, PB1 and PA, with multiple DI bands of PB2 and PA. FIG. 11C is a schematic of PB2 DI bands sub-cloned into pGEM-T easy plasmids, indicating the relative sizes of each of the 5 DI PB2 species (PB2-DI910, PB2-DI751, PB2-DI597, PB2-DI546 and PB2-DI458 deletion mutants, respectively) that were identified by Sanger sequencing.

FIGS. 12A-12E show the rational design of a PB2/PB1 2DI. FIG. 12A is a schematic of a reverse-genetics expression system using H1N1 and WSN co-transfected to MDCK-2P and 293FT-2P to generate a recombinant PB1-DI virus. viral RNA segments from H1N1 (●) and WSN (▴) are shown, respectively. FIG. 12B is an image of a gel confirming the incorporation of PB2-DI548 and PB1-DI477 into the viral genome, respectively, showing molecular size marker for bands in lanes corresponding to PB2, PB1 and PA. FIGS. 12C-E are images of plaque assays of H1N1/WSN/PB2-597/PB1-477 performed in MDCK-2P (FIG. 12C), MDCK-PB2 (FIG. 12D), and MDCK (FIG. 12E) cells, respectively; the assay indicate that only MDCK-2P supports the replication of H1N1/WSN/PB2-597/PB1-477.

FIGS. 13A-13H show the generation of H1N1/PR8 DI. FIGS. 13A-13C are schematics of a reverse-genetics expression system using H7N9 and PR8 co-transfected to MDCK-2P and 293FT-2P to generate the recombinant PB2-DI virus (FIG. 13A), PB1-DI virus (FIG. 13B), and PB2/PB1-DI virus (FIG. 13C), respectively, to generate a recombinant PB1-DI virus; viral RNA segments from H7N9 (●) and H1N1/PR8 (▴) are shown, respectively. FIGS. 13D-13H are images of gels confirming the incorporation of PB2-322 (FIG. 13D), PB2-548 (FIG. 13E), PB2-597 (FIG. 13F), PB2-477 (FIG. 13G), and PB2-597/PB1-477 (FIG. 13H), into the viral genome, respectively, showing molecular size marker for bands in lanes corresponding to PB2, PB1 and PA.

FIGS. 14A-14E show the generation of H1N1/pdm09 DI. FIGS. 14A-14B are schematics of a reverse-genetics expression system using H7N9 and pdm09 co-transfected to MDCK-2P and 293FT-2P to generate the recombinant PB2-DI virus (FIG. 14A), and PB2-DI virus (FIG. 14B), respectively, to generate a recombinant virus; viral RNA segments from H7N9 (●) and H1N1/pdm09 (▴) are shown, respectively. FIGS. 14C-14E are images of gels confirming the incorporation of PB2-548 (FIG. 14C), PB1-952 (FIG. 14D), and PB1-925 (FIG. 14E), into the viral genome, respectively, showing molecular size marker for bands in lanes corresponding to PB2, PB1 and PA.

FIGS. 15A-15D show the generation of H1N1/pdm09 DI. FIGS. 15A-15D are schematics of a reverse-genetics expression system using Hong Kong/4801/14 (H3N2), H7N9 and PR8 co-transfected to SIAT-1 cells and 293FT-2P to generate the recombinant PB2-DI virus (FIG. 15A) having viral RNA segments from H7N9 (●), H3N2 (▪), and H1N1 PR8 (▴), respectively. FIGS. 15B-15D are images of gels confirming the incorporation of H3N2-4801/PB2-548 (FIG. 15B), H3N2-4801/PB1-751 (FIG. 15C), and H3N2-4801/PB2-910 (FIG. 15D), into the viral genome, respectively, showing molecular size marker for bands in lanes corresponding to PB2, PB1 and PA.

FIGS. 16A-16D show the generation of H1N1/pdm09 DI. FIGS. 16A-16D are schematics of a reverse-genetics expression system using Hong Kong/4801/14 (H3N2), H7N9 and WSN co-transfected to MDCK-2P cells and pCAGEN-H7-PA to generate the recombinant PA-DI virus (FIG. 16A) having viral RNA segments from H7N9 (●), H3N2 (▪), and H1N1 WSN (▴), respectively. FIGS. 16B-16D are images of gels confirming the incorporation of H1N1-WSN/PA-416 (FIG. 16B), H1N1-WSN/PA-481 (FIG. 16C), and H1N1-WSN/PA-623 (FIG. 16D), into the viral genome, respectively, showing molecular size marker for bands in lanes corresponding to PB2, PB1 and PA.

### DETAILED DESCRIPTION OF THE INVENTION

### I. Definitions

The term “non-replicating” influenza refers to an influenza virus that is not capable of replication to any significant extent in the majority of normal mammalian cells or normal primary human cells. For example, in some forms, the influenza virus has a replication capability of 5%, 2%, 1%, 0.5%, 0.1% or 0% compared to wild-type influenza virus in standardized assays.

The term “modified” virus refers to an influenza virus that has been altered in some way that changes one or more characteristics of the modified virus compared to the wild-type virus. These changes may have occurred naturally or through engineering.

The terms “influenza virus,” “influenza” and “flu virus” are used interchangeably and refer to the group of influenza virus A, influenza virus B, influenza virus C and influenza virus D. Human influenza A and B viruses cause seasonal epidemics of disease (termed the “flu season”) in humans almost every winter in the United States. Global epidemics of flu disease are termed “Flu pandemics,” and typically occur when a new and very different influenza A virus emerges that both infects humans and has the ability to spread efficiently between humans. Influenza A viruses are categorized as either the hemagglutinin subtype or the neuraminidase subtype based on the proteins involved, and there are 18 distinct subtypes of hemagglutinin and 11 distinct subtypes of neuraminidase. Influenza A is the primary cause of flu epidemics, and they constantly change and are difficult to predict.

The terms “PB2 gene” and “PB2 subunit” refer to the gene which encodes the influenza virus RNA polymerase PB2 component, which is located on segment 1 of the 8-segmented single-stranded influenza RNA genome.

The terms “genomic segment” or “segment,” used in the context of an influenza virus, refer to the eight single-stranded negative sense RNA molecules spanning approximately 13.5 kilobases (kb) that together encompass the influenza virus genome. The segments range in length from 890 to 2,341 nucleotides and encode a total of 11 proteins.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The terms “immunologic,” “immunological” or “immune” response refer to the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “T cell antigen” refers to a protein or fragment thereof which can be processed into a peptide that can bind to either Class I MHC, Class II MHC, non-classical MHC, or CD1 family molecules (collectively antigen presenting molecules), and in this combination can engage a T cell receptor on a T cell. Accordingly, a T cell mediated immune response is a response that occurs as a result of recognition of a T cell antigen bound to an antigen presenting molecule on the cell surface of an antigen presenting cell, coupled with other interactions between costimulatory molecules on the T cell and APC. This response serves to induce T cell proliferation, anatomic migration, and production of effector molecules, including cytokines and other factors that can injure cells.

The term “B cell antigen” refers to a protein, glycoprotein, carbohydrate, or lipid that can bind to cell surface antibody and can generate the production of soluble antibodies. A humoral immune response is the generation of an immune response that leads to high and sustained levels of circulating antibodies.

The terms “treat” or “treatment” of a disease, disorder or condition refer to improving one or more symptoms or the general condition of a subject having the disease. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. In the case of cancer, “treating” the cancer refers to inhibiting proliferation or metastasis of a cancer or tumor cells. In some embodiments, treatment leads to stasis, partial or complete remission of a tumor or inhibit metastatic spreading of the tumor. In the case of an infectious disease, “treating” the infectious disease means reducing the load of the infections agent in the subject. In some embodiments, the load is viral load, and reducing the viral load means, for example, reducing the number of cells infected with influenza virus or coronavirus, reducing the rate of replication of influenza virus or coronavirus, reducing the number of new virions produced or reducing the number of total viral genome copies in a cell, as compared to an untreated subject. In some embodiments, the load is influenza virus, or coronavirus, as compared to an untreated subject, or as compared to a healthy, uninfected subject.

The term “protect” or “protection of” a subject from developing a disease or from becoming susceptible to an infection means to partially or fully protect a subject. The phrase “fully protect” means that a treated subject does not develop a disease or infection caused by an agent such as a virus, bacterium, fungus, protozoa, helminth, and parasites, or caused by a cancer cell. To “partially protect” as used herein means that a certain subset of subjects may be fully protected from developing a disease or infection after treatment, or that the subject does not develop a disease or infection with the same severity as an untreated subject. The term “protective immune response” or “protective immunity” refers to an immune response to an antigen that is sufficient to provide immunological protection against re-exposure to the same or similar antigen, for example, subsequent infection by a pathogenic organism from which the antigen is derived.

The terms “effective amount” or “therapeutically effective amount” mean a dosage or other amount of an active agent that produces some desired effect at a reasonable benefit/risk ratio applicable to any medical treatment or diagnosis. Typically, an amount of an agent is therapeutically effective if it is sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered. One of ordinary skill in the art may empirically determine the effective amount of a particular compound without necessitating undue experimentation.

The terms “pharmaceutically acceptable” or “biocompatible” refer to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; and N-benzylphenethylamine.

The term “biodegradable” generally refers to a material that will degrade or erode under physiological conditions to smaller units or chemical species that are capable of being metabolized, eliminated, or excreted by the body. The degradation time of a material is a function of composition and morphology of the material.

The terms “inhibit” or “reduce” generally mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. In some embodiments, the inhibition and reduction are compared at mRNAs, proteins, cells, tissues and organs levels.

The terms “prevent,” “prevention” or “preventing” mean to administer a composition or method to a subject or a system at risk for or having a predisposition for one or more symptom caused by a disease or disorder, to decrease the likelihood the subject will develop one or more symptoms of the disease or disorder, or to reduce the severity, duration, or time of onset of one or more symptoms of the disease or disorder.

The terms “bioactive agent” and “active agent,” as used interchangeably include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The terms “protein” “polypeptide” or “peptide” refer to a natural or synthetic molecule including two or more amino acids linked by the carboxyl group of one amino acid to the alpha amino group of another.

The term “polynucleotide” or “nucleic acid” or “nucleic acid sequence” refers to a natural or synthetic molecule including two or more nucleotides linked by a phosphate group at 3′ position of one nucleotide to 5′ end of another nucleotide. The polynucleotide is not limited by length, and thus the polynucleotide can include deoxyribonucleic acid (DNA) or ribonucleic acid (RNA).

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

### II. Intra-Dermal Influenza Vaccine (IDIV) Compositions

It has been established that compositions of replication-deficient influenza viruses can provide long-term, cross-protective immunity to influenza viruses with multiple genetic backgrounds, when administered intradermally to epithelial tissue. Compositions of live, replication-deficient influenza viruses for inducing broadly cross-protective T-cell mediated immunity to influenza are described. Since live, replication-deficient influenza viruses are non-replicative in vivo, these reagents allows administration in vivo at high dose with minimal concern about adverse inflammatory effect caused by viral replication.

The compositions include live, replication-deficient influenza viruses, and optionally one or more additional active agents and/or adjuvants. In some embodiments, one or more additional molecules enhances or induces the immune response in the recipient when co-administered with the live, replication-deficient influenza viruses. Exemplary additional molecules include co-stimulatory molecules, growth factors, and cytokines. In some embodiments, the compositions include a pharmaceutically acceptable excipient for administration into the body by intradermal administration.

### A. Replication-Deficient, Intact Influenza Virus

The compositions include a live, intact influenza virus that has been modified to be replication-deficient in normal mammalian cells, such as normal human cells. In preferred forms, the modified influenza virus is capable of infecting normal mammalian cells, such as normal human cells.

The term “modified” influenza virus refers to influenza virus that has been altered in some way that changes one or more characteristics of the modified virus compared to the wild-type virus. These changes may have occurred naturally or through engineering. Typically, the live, intact, non-replicating or replication-impaired influenza virus is an engineered (i.e., recombinant and/or chimeric) virus.

In some forms, the modified virus is a chimeric virus, for example, based on a live influenza A or B virus, engineered to contain one or more mutations that inhibits or prevents viral replication in host cells. Typically, the one or more mutations does not alter or does not substantially alter the structure, infectivity, or antigenicity of the virus in host cells relative to a replication-competent virus lacking the one or more mutations. In some forms, the one or more mutations include deletions within the influenza RNA polymerase PB1 or PB2 subunit gene. Therefore, in some forms, the non-replicating or replication-impaired live influenza virus incudes an influenza genome including a truncated influenza RNA polymerase PB1 and/or PB2 subunit genes. The mutated and/or truncated RNA polymerase PB1 and/or PB2 subunit can be derived from the same, or a different influenza virus. In some forms, the mutated RNA polymerase gene(s) are exogenous gene(s). In other forms, the mutated RNA polymerase gene(s) are derived from the same strain of influenza. An exemplary mutated RNA polymerase PB2 subunit gene is derived from an H7N1 virus or from an H5N1 virus.

The terms “replication-deficient” and “non-replicating” influenza virus refer to an influenza virus that is not capable of replication to any significant extent in the majority of normal mammalian cells or normal primary human cells. The term “significant extent” means a replication capability of 75% or less as compared to wild-type, replication-competent influenza virus in standardized assays. In some embodiments, the influenza virus has a replication capability of 65%, 55%, 45%, 35%, 25%, or 15% compared to wild-type influenza virus. In some embodiments, the virus has a replication capability 10% or less, 5% or less, or 1% or less compared to an infectious, replication-competent wild-type influenza virus. The replication-efficacy of an engineered replication-deficient influenza virus can be compared to that of a control, such as a replication-competent influenza virus in the same host cell. An exemplary control virus is a wild-type influenza virus, for example, a human influenza virus capable of infecting and replicating within human cells in vitro and/or in vivo. An exemplary wild-type influenza virus is a non-modified virus, i.e., lacking the one or more mutations that inhibits replication in the engineered viruses. For example, in some forms, the one or more mutations reduces the replication of the virus in by 65%, 75%, 85%, 95%, 98%, or 99% compared to wild-type influenza virus. Non-replicating viruses are 100% replication deficient in normal primary human cells. The replication deficient, or non-replicating, or replication-impaired influenza virus are intact and viable particles, as opposed to virus that has been physically or chemically-inactivated, for example, by exposure to formalin or β-propiolactone, to destroy infectivity.

Viral replication assays are known in the art, and can be performed for influenza virus on e.g., primary keratinocytes, and are described in the Examples. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as MDCK cells.

In some embodiments, the modified influenza viruses may also have altered characteristics concerning aspects of the viral life cycle, such as target cell specificity, route of infection, rate of infection, rate of replication, rate of virion assembly and/or rate of viral spreading.

Typically, the intact replication-deficient influenza viruses have the same external structure/morphology as wild type viruses. For example, the intact replication-deficient influenza viruses generally exhibit the same, or substantially the same antigenic characteristics, and the same or substantially similar size, shape and mass as the wild-type influenza viruses from which they derive, or which they are engineered to imitate.

### 1. Backbone Influenza Viruses

The intact, non-replicating or replication-impaired influenza virus is typically derived from a “backbone,” replication-competent “wild-type” influenza virus. Exemplary backbone influenza viruses include all pre-existing, replication-competent “wild-type” influenza viruses, for example, an influenza virus subtype, clade and strain known in the art.

The backbone influenza virus is typically one of the four types of influenza viruses: A, B, C and D. Human influenza A and B viruses cause seasonal epidemics of disease in humans. Influenza A viruses are the only influenza viruses known to cause flu pandemics, i.e., global epidemics of flu disease. Influenza type C infections generally cause mild illness and are not thought to cause human flu epidemics Influenza D viruses primarily affect cattle and are not known to infect or cause illness in people (see website cdc.gov/flu/about/viruses/types.htm).

Influenza A viruses are divided into subtypes based on hemagglutinin (H) and neuraminidase (N) proteins on the surface of the virus. There are 18 different hemagglutinin subtypes and 11 different neuraminidase subtypes (H1 through H18, and N1 through N11, respectively). Therefore, in some forms, the live, non-replicating or replication-impaired influenza virus is derived from an influenza virus from any one or more of the H1 through H18 subtypes, including any of the H1, H2, H3, H4, H5, H6, H7, H8, H9, H10, H11, H12, H13, H14, H15, H16, H17 or H18 subtype viruses. In other forms, the live, non-replicating or replication-impaired influenza virus is derived from an influenza virus from any one or more of the N1 through N11 subtypes. While there are potentially 198 different influenza A subtype combinations, only 131 subtypes have been detected in nature. Current subtypes of influenza A viruses that routinely circulate in people include: A (H1N1) and A (H3N2). Therefore, in some forms, the live, non-replicating or replication-impaired influenza virus is derived from an A (H1N1) influenza virus, or an A (H3N2) influenza virus.

Influenza A viruses are further classified into multiple subtypes (e.g., H1N1, or H3N2), while influenza B viruses are classified into one of two lineages: B/Yamagata and B/Victoria. Both influenza A and B viruses can be further classified into specific clades and sub-clades. Clades and sub-clades can be alternatively called “groups” and “sub-groups,” respectively. An influenza clade or group is a further subdivision of influenza viruses (beyond subtypes or lineages) based on the similarity of their HA gene sequences. Clades and subclades are shown on phylogenetic trees as groups of viruses that usually have similar genetic changes (i.e., nucleotide or amino acid changes) and have a single common ancestor represented as a node in the tree. Clades and sub-clades that are genetically different from others are not necessarily antigenically different (i.e., viruses from a specific clade or sub-clade may not have changes that impact host immunity in comparison to other clades or sub-clades).

Circulating influenza A (H1N1) viruses are related to the pandemic 2009 H1N1 virus that emerged in spring of 2009 and caused a flu pandemic (See w.w.w.cdc.gov/flu/about/viruses/types.htm). This virus is known as “A (H1N1) pdm09 virus,” or “2009 H1N1,” and continued to circulate seasonally from 2009 to 2021. These H1N1 viruses have undergone relatively small genetic changes and changes to their antigenic properties over time. Of the influenza viruses that circulate and cause human disease, influenza A (H3N2) viruses tend to change more rapidly, both genetically and antigenically and have formed many separate, genetically different clades that continue to co-circulate. Therefore, in some forms, the live, non-replicating or replication-impaired influenza virus is derived from a currently circulating H1N1 influenza virus.

In other forms, the live, non-replicating or replication-impaired influenza virus is derived from a currently circulating H3N2 influenza virus. In preferred forms, the live, non-replicating or replication-impaired influenza virus is derived from a currently circulating H1N1 influenza virus or H3N2 influenza virus.

Influenza B viruses are classified into two lineages: B/Yamagata and B/Victoria. Influenza B viruses are further classified into specific clades and sub-clades. Influenza B viruses change more slowly in terms of genetic and antigenic properties than influenza A viruses. Surveillance data from recent years shows co-circulation of influenza B viruses from both lineages in the United States and around the world. Therefore, in some forms, the live, non-replicating or replication-impaired influenza virus is derived from an influenza B virus. In some forms, the live, non-replicating or replication-impaired influenza virus is derived from a currently circulating influenza B virus. In some forms, the live, non-replicating or replication-impaired influenza virus is derived from B/Yamagata or B/Victoria influenza viruses.

In some forms the live, non-replicating or replication-impaired influenza virus is derived from one or more zoonotic influenza viruses. Exemplary zoonotic influenza viruses include equine viruses, including equine influenza virus or equine herpesvirus: equine influenza virus type A/Alaska 91, equine influenza virus type A/Miami 63, or equine influenza virus type A/Kentucky 81. Exemplary cattle viruses include bovine parainfluenza virus type 3, and bovine parainfluenza virus type 3.

i. Backbone Influenza Virus Structure

The influenza virus genome is segmented, including 8 different segments of negative-sense, single-stranded viral RNA (vRNA), each coding for at least one of the influenza HA, NA, M2, NS2, NB, PB1, PB2, PA or NP genes. Segment 1 encodes RNA polymerase subunit (PB2); Segment 2 encodes RNA polymerase subunit (PB1) and the PB1-F2 protein, which induces cell death, by using different reading frames from the same RNA segment. Segment 3 encodes RNA polymerase subunit (PA) and the PA-X protein, which has a role in host transcription shutoff; Segment 4 encodes for HA (hemagglutinin); Segment 5 encodes NP, which is a nucleoprotein; Segment 6 encodes NA (neuraminidase); Segment 7 encodes two matrix proteins (M1 and M2); and Segment 8 encodes two distinct non-structural proteins (NS1 and NEP).

Wildtype influenza virions are typically spherical or elliptical shapes with 80-120 nm diameter (Noda, Front Microbiol 2, 269 (2011)), and include the classical influenza antigens hemagglutinin (HA) (See Genbank accession No. JO2132; Air, 1981, Proc. Natl. Acad. Sci. USA 78:7639-7643; Newton, et al., 1983, Virology 128:495-501), and neuraminidase (NA): the influenza A virion is studded with glycoprotein spikes of hemagglutinin (HA) and neuraminidase (NA), in a ratio of approximately four to one, projecting from a host cell-derived lipid membrane. A smaller number of matrix (M2) ion channels traverse the lipid envelope, with an M2: HA ratio on the order of one M2 channel per 101-102 HA molecules. The envelope and its three integral membrane proteins HA, NA, and M2 overlay a matrix of M1 protein, which encloses the virion core. Internal to the M1 matrix are found the nuclear export protein (NEP; also called nonstructural protein 2, NS2) and the ribonucleoprotein (RNP) complex, which includes of the viral RNA segments coated with nucleoprotein (NP) and the heterotrimeric RNA-dependent RNA polymerase, composed of two “polymerase basic” and one “polymerase acidic” subunits (PB1, PB2, and PA). The organization of the influenza B virion is similar, with four envelope proteins: HA, NA, and, instead of M2, NB and BM2.

Generally, the intact, non-replicating or replication-impaired influenza virus is structurally equivalent to the wild-type virus apart from the presence of one or more mutations that inhibit the replication of the virus. In some forms, the live, non-replicating or replication-impaired influenza virus virion is of the same shape and dimensions as a wild-type virion. For example, in some forms, a live, non-replicating or replication-impaired influenza virus virion has a spherical or elliptical shape, with a size of 80-120 nm diameter. Typically, the live, non-replicating or replication-impaired influenza virus includes the same number of RNA “segments” as a wild-type virion. For example, in some forms, a live, non-replicating or replication-impaired influenza virus virion includes 8 different segments of negative-sense, single-stranded viral RNA (vRNA), each coding for all or part of at least one of the influenza HA, NA, M2, NS2, NB, PB1, PB2, PA or NP genes.

### 2. Engineered Non-Replicating Viruses

The modified intact, replication-deficient influenza virus is typically an engineered virus, for example, a recombinant virus including one or more genetic mutations that prevents or substantially inhibits the ability of the virus to replicate in primary human cells. Typically, the engineered non-replicating or replication-impaired influenza virus includes one or more mutations in one or more genes that is necessary for viral replication in host cells.

i. Mutated Genes Inhibiting Replication

Typically, mutations that inhibit or reduce viral replication in normal mammalian cells disrupt the expression, assembly or function of the viral replication machinery, namely the viral RNA polymerase. Therefore, in some forms, the intact replication-deficient influenza viruses include mutations in one or more of the genes that encode the viral RNA polymerase, i.e., the PB2 gene (genome segment 1); PB1 gene (genome segment 2) and the PB1-F2 protein; the PA gene (genome segment 3) or PA-X protein and/or the nucleoprotein (NP) gene (genome-segment 5).

In preferred forms, the non-functional mutation associated with non-replication of the engineered influenza virus is in one or more of the influenza PA, PB1, PB2 or NP genes. In a particularly preferred form, the non-functional mutation associated with non-replication of the engineered influenza virus completely abrogates the function of the RNA viral polymerase. For example, in some forms, the mutation is in the PB2 gene. In some forms, the mutant PB2 gene includes one or more of a substitution, deletion, or addition of one or more amino acids within the PB2 gene that prevents or substantially alters the native function of the PB2 gene. In preferred forms, the one or more mutations in the PB2 gene completely abrogates the function of the viral RNA polymerase without altering the external structure of the virion, such that the influenza virus is infectious but completely non-replicating in primary human cells. An exemplary mutant PB2 segment is a PB2 gene with a nucleic acid sequence that lacks one or more nucleic acids present within the wild-type gene. For example, in some forms the mutated PB2 gene includes a deletion of multiple contiguous nucleic acids at positions from about 121 to about 2138, inclusive of the wild-type gene. The defective PB2 or NP gene is stable, such that the viral gene can be maintained within serial passages of a host cell.

a. Non-functional Mutant PB2 Genes

In some forms, the non-functional mutation associated with non-replication of the engineered influenza virus is derived from the A/H7N9 PB2 segment (segment 1). In some forms, the mutant PB2 segment is a mutated PB2 gene derived from the A/H7N9 influenza virus including a deletion of one or more of the nucleic acids from positions 121-2138 of the wild-type gene.

In some forms, the mutant PB2 segment also includes one or more silent mutations that are substitutions of the amino acids at 5′ and/or 3′ ends of the PB2 gene. For example, in some forms the mutations include one or more substitutions that introduce guanine at position 32, cytosine at position 33, guanine at position 35, cytosine at position 38, guanine at position 41, guanine at position 44, thymine at position 2288, cytosine at position 2289, guanine at position 2291, cytosine at position 2297, cytosine at position 2300 and thymine at position 2303 of the PB2 gene from A/H7N9 influenza virus.

In some forms, the mutated gene PB2 gene from A/H7N9 influenza virus is a truncated, non-functional PB2 gene of 322 nucleotides in length, having the nucleic acid sequence:

(SEQ ID NO: 1)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCGGGCCAGCATTGAGC
ATCAACGAATTGAGCAATCTTGCGAAAGGAGAGAAGGCTAATGTG
TTGATAGGGCAAGGAGACGTTGTGTTGGTGATGAAACGGAAACGG
GACTCTAGCATACTTACTGACAGTCAGACAGCGACCAAAAGGATT
CGGATGGCCATCAATTAATGTCGAATTGTTTAAAAACGACCTTGT
TTCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:1.

In some forms, the mutated gene PB2 gene from A/H7N9 influenza virus is a truncated, non-functional PB2 gene of 546 nucleotides in length (PB2 (H7N9 ZJ)-DI546), having the nucleic acid sequence:

(SEQ ID NO: 9)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCAAGAAATATACATCA
GGAAGACAGGAGAAGAATCCTGCCCTTAGGATGAAGTGGATGATG
GCAATGAAATATCCAATTACGGCAGACAAAAGGATAATGGAGATG
ATCCCGGAAAGAAATGAGCAAGGTCAGACCCTTTGGAGCAAGACA
AATGATGCTGGATCAGACAGAGTGATGGTGTCACCTCGGGATTTC
TGATTCTGGGCAAAGAAAACAAAAGATATGGGCCAGCATTGAGCA
TCAACGAATTGAGCAATCTTGCGAAAGGAGAGAAGGCTAATGTGT
TGATAGGGCAAGGAGACGTTGTGTTGGTGATGAAACGGAAACGGG
ACTCTAGCATACTTACTGACAGTCAGACAGCGACCAAAAGGATTC
GGATGGCCATCAATTAATGTCGAATTGTTTAAAAACGACCTTGTT
TCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:9.

In some forms, the mutated gene PB2 gene from A/H7N9 influenza virus is a truncated, non-functional PB2 gene of 596 nucleotides in length (PB2 (H7N9 ZJ)-DI596, having the nucleic acid sequence:

(SEQ ID NO: 10)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCAAGAAATATACATCA
GGAAGACAGGAGAAGAATCCTGCCCTTAGGATGAAGTGGATGATG
GCAATGAAATATCCAATTACGGCAGACAAAAGGATAATGGAGATG
ATCCCGGAAAGAAATGAGCAAGGTCAGACCCTTTGGAGCAACCAA
GAGGCTTACGGTGCTTGGAAAGGATGCAGGTGCATTGATGGAAGA
CCCCGATGAGGGAACAGCAGGAGTGGAATCTGCGGTATTGAGGGG
ATTTCTGATTCTGGGCAAAGAAAACAAAAGATATGGGCCAGCATT
GAGCATCAACGAATTGAGCAATCTTGCGAAAGGAGAGAAGGCTAA
TGTGTTGATAGGGCAAGGAGACGTTGTGTTGGTGATGAAACGGAA
ACGGGACTCTAGCATACTTACTGACAGTCAGACAGCGACCAAAAG
GATTCGGATGGCCATCAATTAATGTCGAATTGTTTAAAAACGACC
TTGTTTCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:10.

In some forms, the mutated gene PB2 gene from A/H7N9 influenza virus is a truncated, non-functional PB2 gene of 910 nucleotides in length (PB2 (H7N9 ZJ)-DI910), having the nucleic acid sequence:

(SEQ ID NO: 11)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCAAGAAATATACATCA
GGAAGACAGGAGAAGAATCCTGCCCTTAGGATGAAGTGGATGATG
GCAATGAAATATCCAATTACGGCAGACAAAAGGATAATGGAGATG
ATCCCGGAAAGAAATGAGCAAGGTCAGACCCTTTGGAGCAAGACA
AATGATGCTGGATCAGACAGAGTGATGGTGTCACCTCTGGCTGTG
ACGTGGTGGAACAGAAATGGACCAACGACAAGCACAGTCCATTAT
CCAAAGGTCTATAAAACCTATTTTGAAAAGGTCGAAAGGCAGATG
CGTGACGTACTGGGAACATTTGACACTGTCCAAATAATAAAGCTA
TTACCATTTGCAGCAGCCCCGCCGGAGCAGAGTAGGATGCAGTTC
TCTTCTCTAACTGTGAATGTGAGGGGTTCCGGAATGAGAATAGTT
GTGAGAGGCAATTCTCCTGTGTTCAACTACAACAAGGCAACCAAG
AGGCTTACGGTGCTTGGAAAGGATGCAGGTGCATTGATGGAAGAC
CCCGATGAGGGAACAGCAGGAGTGGAATCTGCGGTATTGAGGGGA
TTTCTGATTCTGGGCAAAGAAAACAAAAGATATGGGCCAGCATTG
AGCATCAACGAATTGAGCAATCTTGCGAAAGGAGAGAAGGCTAAT
GTGTTGATAGGGCAAGGAGACGTTGTGTTGGTGATGAAACGGAAA
CGGGACTCTAGCATACTTACTGACAGTCAGACAGCGACCAAAAGG
ATTCGGATGGCCATCAATTAATGTCGAATTGTTTAAAAACGACCT
TGTTTCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:11.

In some forms, the mutated gene PB2 gene from A/H7N9 influenza virus is a truncated, non-functional PB2 gene of 458 nucleotides in length (PB2 (H7N9 ZJ)-DI458), having the nucleic acid sequence:

(SEQ ID NO: 12)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCAAGAAATATACATCA
GGAAGACAGGAGAAGAATCCTGCCCTTAGGATGAAGTGGATGATG
GCAATGAAATATCCAATTACGGCAGACAAAAGGATAATGGAGATG
ATCCCGGAAAGAAATGAGCAAGGTCAGACCCTTTGGAGCAAGACA
AATGATGGAATTGAGCAATCTTGCGAAAGGAGAGAAGGCTAATGT
GTTGATAGGGCAAGGAGACGTTGTGTTGGTGATGAAACGGAAACG
GGACTCTAGCATACTTACTGACAGTCAGACAGCGACCAAAAGGAT
TCGGATGGCCATCAATTAATGTCGAATTGTTTAAAAACGACCTTG
TTTCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:12.

In some forms, the mutated gene PB2 gene from A/H7N9 influenza virus is a truncated, non-functional PB2 gene of 548 nucleotides in length (PB2 (H7N9 ZJ)-DI548), having the nucleic acid sequence:

(SEQ ID NO: 13)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCAAGAAATATACATCA
GGAAGACAGGAGAAGAATCCTGCCCTTAGGATGAAGTGGATGATG
GCAATGAAATATCCAATTACGGCAGACAAAAGGATAATGGAGATG
ATCCCGGAAAGAAATGAGCAAGGTCAGACCCTTTGGAGCAAGACA
AATGATGCTGGATCAGACAGAGTGATGGTGTCACCTCAGGGGATT
TCTGATTCTGGGCAAAGAAAACAAAAGATATGGGCCAGCATTGAG
CATCAACGAATTGAGCAATCTTGCGAAAGGAGAGAAGGCTAATGT
GTTGATAGGGCAAGGAGACGTTGTGTTGGTGATGAAACGGAAACG
GGACTCTAGCATACTTACTGACAGTCAGACAGCGACCAAAAGGAT
TCGGATGGCCATCAATTAATGTCGAATTGTTTAAAAACGACCTTG
TTTCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:13.

In some forms, the mutated gene PB2 gene from A/H7N9 influenza virus is a truncated, non-functional PB2 gene of 597 nucleotides in length (PB2 (H7N9 ZJ)-DI597), having the nucleic acid sequence:

(SEQ ID NO: 14)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCAAGAAATATACATCA
GGAAGACAGGAGAAGAATCCTGCCCTTAGGATGAAGTGGATGATG
GCAATGAAATATCCAATTACGGCAGACAAAAGGATAATGGAGATG
ATCCCGGAAAGAAATGAGCTGTGTTCAACTACAACAAGGCAACCA
AGAGGCTTACGGTGCTTGGAAAGGATGCAGGTGCATTGATGGAAG
ACCCCGATGAGGGAACAGCAGGAGTGGAATCTGCGGTATTGAGGG
GATTTCTGATTCTGGGCAAAGAAAACAAAAGATATGGGCCAGCAT
TGAGCATCAACGAATTGAGCAATCTTGCGAAAGGAGAGAAGGCTA
ATGTGTTGATAGGGCAAGGAGACGTTGTGTTGGTGATGAAACGGA
AACGGGACTCTAGCATACTTACTGACAGTCAGACAGCGACCAAAA
GGATTCGGATGGCCATCAATTAATGTCGAATTGTTTAAAAACGAC
CTTGTTTCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:14.

In some forms, the mutated gene PB2 gene from A/H7N9 influenza virus is a truncated, non-functional PB2 gene of 751 nucleotides in length (PB2 (H7N9 ZJ)-DI751), having the nucleic acid sequence:

(SEQ ID NO: 15)
AGCAAAAGCAGGTCAAATATATTCAATATGGAAAGAATAAAAGAA
CTAAGAGATTTGATGTCACAGTCTCGCACTCGCGAGATACTGACA
AAAACAACTGTGGACCATATGGCCATAATCAAGAAATATACATCA
GGAAGACAGGAGAAGAATCCTGCCCTTAGGATGAAGTGGATGATG
GCAATGAAATATCCAATTACGGCAGACAAAAGGATAATGGAGATG
ATCCCGGAAAGAAATGAGCAAGGTCAGACCCTTTGGAGCAAGACA
AATGATGCTGGATCAGACAGAGTACCATTTGCAGCAGCCCCGCCG
GAGCAGAGTAGGATGCAGTTCTCTTCTCTAACTGTGAATGTGAGG
GGTTCCGGAATGAGAATAGTTGTGAGAGGCAATTCTCCTGTGTTC
AACTACAACAAGGCAACCAAGAGGCTTACGGTGCTTGGAAAGGAT
GCAGGTGCATTGATGGAAGACCCCGATGAGGGAACAGCAGGAGTG
GAATCTGCGGTATTGAGGGGATTTCTGATTCTGGGCAAAGAAAAC
AAAAGATATGGGCCAGCATTGAGCATCAACGAATTGAGCAATCTT
GCGAAAGGAGAGAAGGCTAATGTGTTGATAGGGCAAGGAGACGTT
GTGTTGGTGATGAAACGGAAACGGGACTCTAGCATACTTACTGAC
AGTCAGACAGCGACCAAAAGGATTCGGATGGCCATCAATTAATGT
CGAATTGTTTAAAAACGACCTTGTTTCTACT.

In some forms, the mutated gene PB2 gene from A/H7N9 influenza virus has a nucleic acid sequence that is at least 70%, at least 80%, at least 90%, at least 95% at least 98%, or at least 99% identical to SEQ ID NO:15.

### B. Non-Functional Mutant PA Genes

In some forms, the non-functional mutation associated with non-replication of the engineered influenza virus is derived from the A/H7N9 PA segment (segment 3). In some forms, the mutant PA segment is a mutated PA gene derived from the A/H7N9 influenza virus including a deletion of the nucleic acids from the wild-type gene.

In some forms, the mutated gene PA gene from A/H7N9 influenza virus is a truncated, non-functional PA gene of 416 nucleotides in length (PA (H7N9 ZJ)-DI416), having the nucleic acid sequence:

(SEQ ID NO: 16)
AGCAAAAGCAGGTACTGATCCAAAATGGAAGACTTTGTGCGACAG
TGCTTCAATCCAATGATCGTCGAGCTTGCGGAAAAGGCAATGAAA
GAATATGGGGAAGATCCGAAAATCGAAACAAACAAATTCGCATCA
ATATGTATTCAACAGTCTATATGCATCTCCGCAACTCGAGGGGTT
CTCAGCTGAATCGAGAAAACTGCTACTCATTGTTCAGGCGCTTAG
GGATAACCTGGAACCTGGAACCTTCGATCTTGAAGGGCTATATGA
AGCAATCGAGGAGTGCCTGATTAATGATCCCTGGGTTTTGCTTAA
TGCATCTTGGTTCAACTCCTTCCTCACACATGCACTAAGATAGTT
GTGGCAATGCTACTATTTGCTATCCATACTGTCCAAAAAAGTACC
TTGTTTCTACT.

In some forms, the mutated gene PA gene from A/H7N9 influenza virus has a nucleic acid sequence that is at least 70%, at least 80%, at least 90%, at least 95% at least 98%, or at least 99% identical to SEQ ID NO:16.

In some forms, the mutated gene PA gene from A/H7N9 influenza virus is a truncated, non-functional PA gene of 481 nucleotides in length (PA (H7N9 ZJ)-DI481), having the nucleic acid sequence:

(SEQ ID NO: 17)
AGCAAAAGCAGGTACTGATCCAAAATGGAAGACTTTGTGCGACAA
TGCTTCAATCCAATGATCGTCGAGCTTGCGGAAAAGGCAATGAAA
GAATATGGGGAAGATCCGAAAATCGAAACAAACAAATTCGCATCA
ATATGCACACACTTAGAAGTCTGCTTCATGTACTCTGATTTCCAC
TGCAGAACCTTACTAGCAAAATCTGTATTCAACAGTCTATATGCA
TCTCCGCAACTCGAGGGGTTCTCAGCTGAATCGAGAAAACTGCTA
CTCATTGTTCAGGCGCTTAGGGATAACCTGGAACCTGGAACCTTC
GATCTTGAAGGGCTATATGAAGCAATCGAGGAGTGCCTGATTAAT
GATCCCTGGGTTTTGCTTAATGCATCTTGGTTCAACTCCTTCCTC
ACACATGCACTAAGATAGTTGTGGCAATGCTACTATTTGCTATCC
ATACTGTCCAAAAAAGTACCTTGTTTCTACT.

In some forms, the mutated gene PA gene from A/H7N9 influenza virus has a nucleic acid sequence that is at least 70%, at least 80%, at least 90%, at least 95% at least 98%, or at least 99% identical to SEQ ID NO:17.

In some forms, the mutated gene PA gene from A/H7N9 influenza virus is a truncated, non-functional PA gene of 506 nucleotides in length (PA (H7N9 ZJ)-DI506), having the nucleic acid sequence:

(SEQ ID NO: 18)
AGCAAAAGCAGGTACTGATCCAAAATGGAAGACTTTGTGCGACAA
TGCTTCAATCCAATGATCGTCGAGCTTGCGGAAAAGGCAATGAAA
GAATATGGGGAAGATCCGAAAATCGAAACAAACAAATTCGCATCA
ATATGCACACACTTAGAAGTCTGCTTCATGTACTCTGATTGGAGT
GGAGGAAGGTTCCATCGGGAAGGTGTGCAGAACCTTACTAGCAAA
ATCTGTATTCAACAGTCTATATGCATCTCCGCAACTCGAGGGGTT
CTCAGCTGAATCGAGAAAACTGCTACTCATTGTTCAGGCGCTTAG
GGATAACCTGGAACCTGGAACCTTCGATCTTGAAGGGCTATATGA
AGCAATCGAGGAGTGCCTGATTAATGATCCCTGGGTTTTGCTTAA
TGCATCTTGGTTCAACTCCTTCCTCACACATGCACTAAGATAGTT
GTGGCAATGCTACTATTTGCTATCCATACTGTCCAAAAAAGTACC
TTGTTTCTACT.

In some forms, the mutated gene PA gene from A/H7N9 influenza virus has a nucleic acid sequence that is at least 70%, at least 80%, at least 90%, at least 95% at least 98%, or at least 99% identical to SEQ ID NO:18.

In some forms, the mutated gene PA gene from A/H7N9 influenza virus is a truncated, non-functional PA gene of 623 nucleotides in length (PA (H7N9 ZJ)-DI623), having the nucleic acid sequence:

(SEQ ID NO: 19)
AGCAAAAGCAGGTACTGATCCAAAATGGAAGACTTTGTGCGACAA
TGCTTCAATCCAATGATCGTCGAGCTTGCGGAAAAGGCAATGAAA
GAATATGGGGAAGATCCGAAAATCGAAACAAACAAATTCGCATCA
ATATGCACACACTTAGAAGTCTGCTTCATGTACTCTGATTTCCAC
TTCATCGACGAACGAGGCGAATCAACTATTATAGAATCTGGCGAT
CCAAATGTGCTGCTGAAACATCTTTGAAAACAAATCTGAAACATG
GCCAATTGGAGAATCACCCAAAGGAGTGGAGGAAGGTTCCATCGG
GAAGGTGTGCAGAACCTTACTAGCAAAATCTGTATTCAACAGTCT
ATATGCATCTCCGCAACTCGAGGGGTTCTCAGCTGAATCGAGAAA
ACTGCTACTCATTGTTCAGGCGCTTAGGGATAACCTGGAACCTGG
AACCTTCGATCTTGAAGGGCTATATGAAGCAATCGAGGAGTGCCT
GATTAATGATCCCTGGGTTTTGCTTAATGCATCTTGGTTCAACTC
CTTCCTCACACATGCACTAAGATAGTTGTGGCAATGCTACTATTT
GCTATCCATACTGTCCAAAAAAGTACCTTGTTTCTACT.

In some forms, the mutated gene PA gene from A/H7N9 influenza virus has a nucleic acid sequence that is at least 70%, at least 80%, at least 90%, at least 95% at least 98%, or at least 99% identical to SEQ ID NO:19.

### C. Non-Functional Mutant PB1 Genes

In some forms, the non-functional mutation associated with non-replication of the engineered influenza virus is derived from the A/H7N9 PB1 segment (segment 2). In some forms, the mutant PB1 segment is a mutated PB1 gene derived from the A/H7N9 influenza virus including a deletion of the nucleic acids from the wild-type gene.

In some forms, the mutated gene PB1 gene from A/H7N9 influenza virus is a truncated, non-functional PB1 gene of 458 nucleotides in length (PB1 (H7N9 ZJ)-DI458), having the nucleic acid sequence:

(SEQ ID NO: 20)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACAAGGAATCGCTCCATTCTCAACACCAGCCAAAG
GGGGATTCTTGAGGACGAACAGATGTACCAGAAGTGCTGCAACCT
ATTCGAAAAGTTCTTCCCCAGCAGTTCGTACAGGAGGCCAGTTGG
AATTTCCAGCATGGTGGAGGCCATGGTGTCTAGGGCCCGAATTGA
TGCACGAATTGACTTCGAATCTGGAAGGATTAAGAAAGAAGAGTT
CGCTGAGATCATGAAGATCTGTTCCACCATTGAAGAGCTCAGACG
GCAAAAATAGTGAATTTAGCTTGTCCTTCATGAAAAAATGCCTTG
TTTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:20.

In some forms, the mutated gene PB1 gene from A/H7N9 influenza virus is a truncated, non-functional PB1 gene of 470 nucleotides in length (PB1 (H7N9 ZJ)-DI470), having the nucleic acid sequence:

(SEQ ID NO: 21)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACACAGTCAACAGAACACATAAATACTCAGAAAAA
GGAAAGTGGACAACGAATTCTTGAGGACGAACAGATGTACCAGAA
GTGCTGCAACCTATTCGAAAAGTTCTTCCCCAGCAGTTCGTACAG
GAGGCCAGTTGGAATTTCCAGCATGGTGGAGGCCATGGTGTCTAG
GGCCCGAATTGATGCACGAATTGACTTCGAATCTGGAAGGATTAA
GAAAGAAGAGTTCGCTGAGATCATGAAGATCTGTTCCACCATTGA
AGAGCTCAGACGGCAAAAATAGTGAATTTAGCTTGTCCTTCATGA
AAAAATGCCTTGTTTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:21.

In some forms, the mutated gene PB1 gene from A/H7N9 influenza virus is a truncated, non-functional PB1 gene of 477 nucleotides in length (PB1 (H7N9 ZJ)-DI477), having the nucleic acid sequence:

(SEQ ID NO: 22)
AGCAAAAGCAGGCAAACCATTTGAATGGATGTCAATCCGACTTTA
CTTTTCTTGCCAGTGCAAAATGCTATAAGTACCACTTTCCCTTAT
ACTGGAGACCCTCCATACAGCCATGGAACAGGAACAGGATACACC
ATGGACACAGTCAACAGAACACATAAATACTCAGAAAAAGGAAAG
TGGACAACGAACACAGAGACTGGAGCACCCCAACTCAATCCAATT
ACCAGAAGTGCTGCAACCTATTCGAAAAGTTCTTCCCCAGCAGTT
CGTACAGGAGGCCAGTTGGAATTTCCAGCATGGTGGAGGCCATGG
TGTCTAGGGCCCGAATTGATGCACGAATTGACTTCGAATCTGGAA
GGATTAAGAAAGAAGAGTTCGCTGAGATCATGAAGATCTGTTCCA
CCATTGAAGAGCTCAGACGGCAAAAATAGTGAATTTAGCTTGTCC
TTCATGAAAAAATGCCTTGTTTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:22.

In some forms, the mutated gene PB1 gene from A/H7N9 influenza virus is a truncated, non-functional PB1 gene of 494 nucleotides in length (PB1 (H7N9 ZJ)-DI494), having the nucleic acid sequence:

(SEQ ID NO: 23)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACACAGTCAACAGAACACATAAATACTCAGAAAAA
GGAAAGTGGACAACGAACACAGAGACTGGAGCACCCCAACTTTCT
TGAGGACGAACAGATGTACCAGAAGTGCTGCAACCTATTCGAAAA
GTTCTTCCCCAGCAGTTCGTACAGGAGGCCAGTTGGAATTTCCAG
CATGGTGGAGGCCATGGTGTCTAGGGCCCGAATTGATGCACGAAT
TGACTTCGAATCTGGAAGGATTAAGAAAGAAGAGTTCGCTGAGAT
CATGAAGATCTGTTCCACCATTGAAGAGCTCAGACGGCAAAAATA
GTGAATTTAGCTTGTCCTTCATGAAAAAATGCCTTGTTTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:23.

In some forms, the mutated gene PB1 gene from A/H7N9 influenza virus is a truncated, non-functional PB1 gene of 690 nucleotides in length (PB1 (H7N9 ZJ)-DI690), having the nucleic acid sequence:

(SEQ ID NO: 24)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACACAGTCAACAGAACACATAAATACTCAGAAAAA
GGAAAGTGGACAACGAACACAGAGACTGGAGCACCCCAACTCAAT
CCAATTGATGGACCATTACCCGAGGACAACGAGCCGAGTGGGTAT
GCACAAACGGATTGTGTATTGGAAGCAATGGCTTTCCTTGAAGAA
TCTCACCCAGGGATCTTTGAAAACTCGTGTCTCGAAACGATGGAA
ATTGTTCAGCAAACAAGAGTGGATAAACTGACCCAAGGCCGCCAG
ACCTATGACTGGACGTTGAATAGAAATCAGCCGGCTGCTACCGCA
TTGGCCAACACTATAGAGGTATTCAGATCGAATGGCCTGACAGCC
AATGAATCAGGAAGGTTGATCGATTTCCTCAAGGACGTGATGGAT
TCAATGGATAAGGAAGAAATGGAGATTACAACACATTTCCAGAGG
AAGAGTTCGCTGAGATCATGAAGATCTGTTCCACCATTGAAGAGC
TCAGACGGCAAAAATAGTGAATTTAGCTTGTCCTTCATGAAAAAA
TGCCTTGTTTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:24.

In some forms, the mutated gene PB1 gene from A/H7N9 influenza virus is a truncated, non-functional PB1 gene of 808 nucleotides in length (PB1 (H7N9 ZJ)-DI808), having the nucleic acid sequence:

(SEQ ID NO: 25)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACACAGTCAACAGAACACATAAATACTCAGAAAAA
GGAAAGTGGACAACGAACACAGAGACTGGAGCACCCCAACTCAAT
CCAATTGATGGACCATTACCCGAGGACAACGAGCCGAGTGGGTAT
GCACAAACGGATTGTGTATTGGAAGCAATGGCTTTCCTTGAAGAA
TCTCACCCAGGGATCTTTGAAAACTCGTGTCTCGAAACGATGGAA
ATTGTTCAGCAAACAAGAGTGGATAAACTGACCCAAGGCCGCCAG
ACCTATGACTGGACGTTGAATAGAAATCAGCCGGCTGCTACCGCA
TTGGCCAACACTATAGAGGTATTCAGATCGAATGGCCTGACAGCC
AATGAATCAGGAAGGTTGATCGATTTCCTCAAGGACGTGATGGAT
TCAATGGATAAGGAAGAAATGGAGATTACAACACATTTCGAAAAG
TTCTTCCCCAGCAGTTCGTACAGGAGGCCAGTTGGAATTTCCAGC
ATGGTGGAGGCCATGGTGTCTAGGGCCCGAATTGATGCACGAATT
GACTTCGAATCTGGAAGGATTAAGAAAGAAGAGTTCGCTGAGATC
ATGAAGATCTGTTCCACCATTGAAGAGCTCAGACGGCAAAAATAG
TGAATTTAGCTTGTCCTTCATGAAAAAATGCCTTGTTTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:25.

In some forms, the mutated gene PB1 gene from A/H7N9 influenza virus is a truncated, non-functional PB1 gene of 925 nucleotides in length (PB1 (H7N9 ZJ)-DI925), having the nucleic acid sequence:

(SEQ ID NO: 26)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACACAGTCAACAGAACACATAAATACTCAGAAAAA
GGAAAGTGGACAACGAACACAGAGACTGGAGCACCCCAACTCAAT
CCAATTGATGGACCATTACCCGAGGACAACGAGCCGAGTGGGTAT
GCACAAACGGATTGTGTATTGGAAGCAATGGCTTTCCTTGAAGAA
TCTCACCCAGGGATCTTTGAAAACTCGTGTCTCGAAACGATGGAA
ATTGTTCAGCAAACAAGAGTGGATAAACTGACCCAAGGCCGCCAG
ACCTATGACTGGACGTTGAATAGAAATCAGCCGGCTGCTACCGCA
TTGGCCAACACTATAGAGGTATTCAGATCGAATGGCCTGACAGCC
AATGAATCAGGAAGGTTGATCGATTTCCTCAAGGACGTGATGGAT
TCAATGGATAAGGAAGAAATGGAGATTACAACACATTTCCAGAGG
AAGAGGAGAGTGAGGGACAACATGACCAAGAAAATGGTCACACAG
AGAACAATAGGAAAGAAAAAACAAAGACTGAACAAAAGGAGCTAC
CTAATAAGAGCACTGACATTGAACACAATGACAAAGGATGCTGAA
AGAGGCAAGCTGAAAAGGAGGGCAATCGCAACACCCGGGATGCAA
ATCAGAGGATTCGTGTATTTTGTAGAAGCACTAGCGAGGAGCATC
TGTGAGAAACTTGAGTTCGCTGAGATCATGAAGATCTGTTCCACC
ATTGAAGAGCTCAGACGGCAAAAATAGTGAATTTAGCTTGTCCTT
CATGAAAAAATGCCTTGTTTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:26.

In some forms, the mutated gene PB1 gene from A/H7N9 influenza virus is a truncated, non-functional PB1 gene of 952 nucleotides in length (PB1 (H7N9 ZJ)-DI952), having the nucleic acid sequence:

(SEQ ID NO: 27)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACACAGTCAACAGAACACATAAATACTCAGAAAAA
GGAAAGTGGACAACGAACACAGAGACTGGAGCACCCCAACTCAAT
CCAATTGATGGACCATTACCCGAGGACAACGAGCCGAGTGGGTAT
GCACAAACGGATTGTGTATTGGAAGCAATGGCTTTCCTTGAAGAA
TCTCACCCAGGGATCTTTGAAAACTCGTGTCTCGAAACGATGGAA
ATTGTTCAGCAAACAAGAGTGGATAAACTGACCCAAGGCCGCCAG
ACCTATGACTGGACGTTGAATAGAAATCAGCCGGCTGCTACCGCA
TTGGCCAACACTATAGAGGTATTCAGATCGAATGGCCTGACAGCC
AATGAATCAGGAAGGTTGATCGATTTCCTCAAGGACGTGATGGAT
TCAATGGATAAGGAAGAAATGGAGATTACAACACATTTCCAGAGG
AAGAGGAGAGTGAGGGACAACATGACCAAGAAAATGGTCACACAG
AGAACAATAGGAAAGAAAAAACAAAGACTGAACAAAAGGAGCTAC
CTAATAAGAGCACTGACATTGAACACAATGACAAAGGATGCTGAA
AGAGGCAAGCTGAAAAGGAGGGCAATCGCAACACCCGGGATGCAA
ATCAGAGGATTCGTGTATTTTGTAGAAGCACTAGCGAGGAGCATC
TGTGAGAAACTTGAGCAATCTGGAAGGATTAAGAAAGAAGAGTTC
GCTGAGATCATGAAGATCTGTTCCACCATTGAAGAGCTCAGACGG
CAAAAATAGTGAATTTAGCTTGTCCTTCATGAAAAAATGCCTTGT
TTCTACT.

In some forms, the mutated gene PB1 gene from A/H7N9 influenza virus has a nucleic acid sequence that is at least 70%, at least 80%, at least 90%, at least 95% at least 98%, or at least 99% identical to SEQ ID NO:27.

ii. Chimeric Non-Replicating Influenza Viruses

In some forms the live, replication-deficient influenza virus is a chimeric virus. Chimeric influenza viruses include a combination of genetic components from two or more different influenza viruses, such as two wild-type influenza viruses. In preferred forms, the chimeric replication-deficient influenza virus includes an influenza genome having one or more of the genes from one or more “donor” virus, and a majority (e.g., four to seven) genes from a “backbone” virus. In some forms, the one or more genes from the donor virus includes one or more mutations that impact the replication efficacy of the chimeric virus.

Therefore, in some forms, live, chimeric, replication-deficient influenza viruses include between one and seven gene segments from a first influenza virus, and between one and seven gene segments from a second influenza virus.

In some forms, the chimeric replication-deficient influenza virus includes five genomic segments, typically including genome segments 4, 5, 6, 7 and 8, which include the viral coat, capsid, M2, NS2, NB, NP, NA, HA, matrix and NS genes, from a first backbone influenza virus, and three genomic segments, typically genome segments 1, 2 and 3, which include the viral RNA polymerase PA, PB1 and PB2 genes, from a second virus, where the segments 1, 2 and/or 3 include one or more mutated PA, PB1 and/or PB2 genes that inhibit or reduce viral replication in normal mammalian cells as compared to a wild-type influenza virus.

In some forms, the live, chimeric, replication-deficient influenza viruses include gene segments from three or more different influenza viruses. For example, in some forms, the live, chimeric, replication-deficient influenza viruses include between one and seven gene segments from a first influenza virus, and between one and seven gene segments from a second influenza virus, and between one and seven gene segments from a third of further influenza virus. Typically, the live chimeric replication-deficient influenza viruses include a total of eight gene segments.

Typically, the first and second, or further influenza backbone viruses are wild-type influenza viruses, for example, influenza virus subtypes, clades and strains known in the art. The gene segments can be derived from a particular influenza clade or strain, or can be synthetic genes, designed to correspond with highly conserved genes amongst multiple different influenza virus strains. Generally, the chimeric replication-deficient influenza viruses include at least one gene from a first virus that is mutated or altered to inhibit the replication ability of the chimeric virus.

a. Exemplary Chimeric H7N9/H1N1 Virus

An exemplary chimeric replication-deficient influenza virus includes the backbone (viral coat, capsid, M2, NS2, NB, NP, NA, HA, matrix and NS genes) from A/WSN/1933 (H1N1), and includes the viral RNA polymerase genes (PB1, PB2 and PA genes) from an H7N9 virus, whereby one or more of the polymerase genes include one or more mutations that abrogate the function of the viral RNA polymerase and reduce or prevent viral replication in normal human cells. In preferred forms, the chimeric replication-deficient influenza virus includes deletions in the PB2 gene located in the viral genome segment 1.

Therefore, an exemplary intact, non-replicating virus has an influenza genome including a mutant PB2 gene from an H7N9 having the nucleic acid sequence of SEQ ID NO: 1; and
a wild-type PB1 gene from an H7N9 virus, having the nucleic acid sequence of:

(SEQ ID NO: 2)
AGCAAAAGCAGGCAAACCATTTGAATGGATGTCAATCCGACTTTA
CTTTTCTTGAAAGTGCCAGTGCAAAATGCTATAAGTACCACTTTC
CCTTATACTGGAGACCCTCCATACAGCCATGGAACAGGAACAGGA
TACACCATGGACACAGTCAACAGAACACATAAATACTCAGAAAAA
GGAAAGTGGACAACGAACACAGAGACTGGAGCACCCCAACTCAAT
CCAATTGATGGACCATTACCCGAGGACAACGAGCCGAGTGGGTAT
GCACAAACGGATTGTGTATTGGAAGCAATGGCTTTCCTTGAAGAA
TCTCACCCAGGGATCTTTGAAAACTCGTGTCTCGAAACGATGGAA
ATTGTTCAGCAAACAAGAGTGGATAAACTGACCCAAGGCCGCCAG
ACCTATGACTGGACGTTGAATAGAAATCAGCCGGCTGCTACCGCA
TTGGCCAACACTATAGAGGTATTCAGATCGAATGGCCTGACAGCC
AATGAATCAGGAAGGTTGATCGATTTCCTCAAGGACGTGATGGAT
TCAATGGATAAGGAAGAAATGGAGATTACAACACATTTCCAGAGG
AAGAGGAGAGTGAGGGACAACATGACCAAGAAAATGGTCACACAG
AGAACAATAGGAAAGAAAAAACAAAGACTGAACAAAAGGAGCTAC
CTAATAAGAGCACTGACATTGAACACAATGACAAAGGATGCTGAA
AGAGGCAAGCTGAAAAGGAGGGCAATCGCAACACCCGGGATGCAA
ATCAGAGGATTCGTGTATTTTGTAGAAGCACTAGCGAGGAGCATC
TGTGAGAAACTTGAGCAATCTGGCCTCCCTGTCGGAGGGAATGAG
AAGAAAGCTAAATTGGCAAATGTTGTAAGGAAGATGATGACTAAT
TCACAAGATACAGAGCTCTCCTTCACAATTACTGGGGACAACACC
AAATGGAATGAGAATCAAAACCCCCGGATGTTTCTAGCAATGATA
ACATACATCACAAGAAACCAGCCAGAATGGTTTAGAAATGTCTTA
AGCATTGCTCCTATAATGTTCTCAAACAAGATGGCGAGATTAGGA
AAAGGGTACATGTTCGAAAGTAAGAGTATGAAGTTACGGACACAA
GTACCAGCGGAAATGCTCGCAAATATTGACCTGAAATACTTCAAC
AAATCAACAAGAGAGAAAATCGAGAAAATAAGACCTCTACTGATA
GATGGCACAGCCTCATTGAGTCCTGGAATGATGATGGGCATGTTC
AACATGTTGAGTACAGTCTTAGGAGTTTCAATTCTGAATCTCGGG
CAGAAGAAGTACACCAAAACCACATATTGGTGGGACGGACTCCAA
TCCTCAGATGACTTCGCCCTCATAGTGAATGCACCGAATCATGAG
GGAATACAGGCAGGAGTAGATAGGTTCTATAGAACCTGCAAATTA
GTTGGGATAAACATGAGCAAGAAGAAATCCTACATAAATCGGACA
GGAACATTCGAATTCACAAGCTTTTTCTACCGCTATGGATTCGTA
GCTAACTTCAGTATGGAGTTGCCCAGTTTTGGAGTGTCCGGGATT
AATGAGTCAGCTGACATGAGCGTTGGTGTTACAGTAATAAAGAAC
AATATGATAAACAACGATCTTGGACCAGCAACAGCCCAAATGGCC
CTTCAGCTATTTATCAAAGACTACAGATACACATACCGATGTCAC
AGGGGTGATACGCAAATTCAAACGAGGAGAGCATTCGAGCTGAAG
AAGCTGTGGGAGCAGACCCGTTCGAAGGCAGGACTGTTGGTTTCA
GATGGAGGGCCAAACCTGTACAATATCCGGAACCTCCACATTCCA
GAGGTCTGCTTGAAATGGGAATTGATGGATGAAGACTACCAAGGC
AGGTTGTGTAATCCTATGAACCCGTTTGTCAGTCATAAGGAAATT
GATTCAGTCAACAATGCTGTGGTGATGCCAGCTCATGGCCCAGCC
AAAAGCATGGAGTATGATGCCGTTGCAACCACACATTCATGGATT
CCTAAGAGGAATCGCTCCATTCTCAACACCAGCCAAAGGGGGATT
CTTGAGGACGAACAGATGTACCAGAAGTGCTGCAACCTATTCGAA
AAGTTCTTCCCCAGCAGTTCGTACAGGAGGCCAGTTGGAATTTCC
AGCATGGTGGAGGCCATGGTGTCTAGGGCCCGAATTGATGCACGA
ATTGACTTCGAATCTGGAAGGATTAAGAAAGAAGAGTTCGCTGAG
ATCATGAAGATCTGTTCCACCATTGAAGAGCTCAGACGGCAAAAA
TAGTGAATTTAGCTTGTCCTTCATGAAAAAATGCCTTGTTTCTACT;

and/or
a wild-type PA gene from an H7N9 virus, having the nucleic acid sequence of:

(SEQ ID NO: 3)
AGCAAAAGCAGGTACTGATCCAAAATGGAAGACTTTGTGCGACAA
TGCTTCAATCCAATGATCGTCGAGCTTGCGGAAAAGGCAATGAAA
GAATATGGGGAAGATCCGAAAATCGAAACAAACAAATTCGCATCA
ATATGCACACACTTAGAAGTCTGCTTCATGTACTCTGATTTCCAC
TTCATCGACGAACGAGGCGAATCAACTATTATAGAATCTGGCGAT
CCAAATGTGCTGCTGAAACACCGATTTGAAATAATCGAAGGGAGA
GACCGAACAATGGCCTGGACAGTGGTGAATAGTATCTGCAACACC
ACGGGAGTCGAAAAACCCAAATTTCTCCCGGATCTGTATGACTAC
AAGGAAAACCGATTCATTGAAATTGGAGTGACGAGGAGGGAAGTC
CACATATATTACCTAGAGAAAGCCAACAAAATAAAATCCGAGAAG
ACACACATCCACATTTTCTCATTCACTGGAGAAGAGATGGCCACC
AAAGCAGATTACACTCTTGACGAAGAAAGCAGGGCAAGAATCAAA
ACCAGGCTGTTCACCATAAGGCAGGAAATGGCCAGCAGGGGTCTA
TGGGATTCCTTTCGTCAGTCTGAAAGAGGCGAAGAAACAATTGAA
GAGAGATTTGAAATCACAGGAACCATGCGCAGGCTTGCCGACCAA
AGTCTCCCACCGAACTTCTCCAGCCTTGAAAACTTTAGAGCCTAT
GTGGATGGATTCGAACCGAACGGCTGCATTGAGGGCAAGCTTTCT
CAAATGTCAAAAGAAGTGAACGCCAGAATCGAGCCATTTCTAAGG
ACAACACCACGCCCTCTCAGATTGCCTGATGGGCCTCCCTGCTCT
CAGCGGTCGAAATTCTTGCTGATGGATGCTCTGAAATTAAGCATT
GAGGACCCGAGCCACGAAGGGGAGGGGATACCGCTATATGATGCG
ATCAAATGCATGAAAACGTTCTTCGGGTGGAAGGAGCCCAACATT
ATCAAACCACATGAGAAAGGCATAAACCCCAATTATCTCCTGACT
TGGAAGCAGGTGCTAGCAGAACTTCAGGACATTGAAAATGAAGAG
AAGATTCCAAGGACAAAGAACATGAAGAAAACAAGCCAATTAAAG
TGGGCACTCGGTGAGAACATGGCACCGGAGAAGGTGGACTTTGAG
GATTGCAAAGATGTCAACGACCTGAAACAGTACGACAGTGATGAG
CCAGAGCCCAGATCACTAGCATGTTGGATCCAGAGTGAATTCAAC
AAGGCATGTGAATTGACTGATTCAAGCTGGGTAGAACTTGATGAA
ATAGGGGAAGATGTTGCTCCAATCGAACACATTGCAAGCATGAGA
CGGAACTATTTCACAGCAGAGGTGTCCCACTGCAGGGCTACTGAA
TATATAATGAAGGGAGTGTACATAAATACAGCTTTGCTCAATGCA
TCTTGTGCAGCCATGGATGACTTTCAACTGATCCCAATGATAAGT
AAATGCAGAACCAAAGAAGGAAGACGGAAAACAAACCTATATGGA
TTCATTATAAAAGGAAGGTCTCACTTGAGGAATGATACTGACGTG
GTAAACTTTGTAAGTATGGAATTTTCCCTTACCGACCCAAGGTTG
GAACCACATAAATGGGAAAAGTATTGTGTTCTTGAAATAGGGGAC
ATGCTCCTGCGAACTGCAGTAGGCCAAGTGTCTAGACCCATGTTT
CTGTATGTGAGAACCAATGGGACCTCCAAGATCAAGATGAAATGG
GGTATGGAAATGAGACGCTGCCTTCTTCAATCTCTCCAACAGATT
GAGAGCATGATTGAAGCTGAATCCTCCGTCAAAGAGAAAGACCTG
ACCAAGGAATTCTTTGAAAACAAATCTGAAACATGGCCAATTGGA
GAATCACCCAAAGGAGTGGAGGAAGGTTCCATCGGGAAGGTGTGC
AGAACCTTACTAGCAAAATCTGTATTCAACAGTCTATATGCATCT
CCGCAACTCGAGGGGTTCTCAGCTGAATCGAGAAAACTGCTACTC
ATTGTTCAGGCGCTTAGGGATAACCTGGAACCTGGAACCTTCGAT
CTTGAAGGGCTATATGAAGCAATCGAGGAGTGCCTGATTAATGAT
CCCTGGGTTTTGCTTAATGCATCTTGGTTCAACTCCTTCCTCACA
CATGCACTAAGATAGTTGTGGCAATGCTACTATTTGCTATCCATA
CTGTCCAAAAAAGTACCTTGTTTCTACT;

and/or
a wild-type HA gene from an H1N1 virus, having the nucleic acid sequence of:

(SEQ ID NO: 4)
AGCAAAAGCAGGGGAAAATAAAAACAACCAAAATGAAGGCAAAAC
TACTGGTCCTGTTATATGCATTTGTAGCTACAGATGCAGACACAA
TATGTATAGGCTACCATGCGAACAACTCAACCGACACTGTTGACA
CAATACTCGAGAAGAATGTGGCAGTGACACATTCTGTTAACCTGC
TCGAAGACAGCCACAACGGGAAACTATGTAAATTAAAAGGAATAG
CCCCACTACAATTGGGGAAATGTAACATCACCGGATGGCTCTTGG
GAAATCCAGAATGCGACTCACTGCTTCCAGCGAGATCATGGTCCT
ACATTGTAGAAACACCAAACTCTGAGAATGGAGCATGTTATCCAG
GAGATCTCATCGACTATGAGGAACTGAGGGAGCAATTGAGCTCAG
TATCATCATTAGAAAGATTCGAAATATTTCCCAAGGAAAGTTCAT
GGCCCAACCACACATTCAACGGAGTAACAGTATCATGCTCCCATA
GGGGAAAAAGCAGTTTTTACAGAAATTTGCTATGGCTGACGAAGA
AGGGGGATTCATACCCAAAGCTGACCAATTCCTATGTGAACAATA
AAGGGAAAGAAGTCCTTGTACTATGGGGTGTTCATCACCCGTCTA
GCAGTGATGAGCAACAGAGTCTCTATAGTAATGGAAATGCTTATG
TCTCTGTAGCGTCTTCAAATTATAACAGGAGATTCACCCCGGAAA
TAGCTGCAAGGCCCAAAGTAAGAGATCAACATGGGAGGATGAACT
ATTACTGGACCTTGCTAGAACCCGGAGACACAATAATATTTGAGG
CAACTGGTAATCTAATAGCACCATGGTATGCTTTCGCACTGAGTA
GAGGGTTTGAGTCCGGCATCATCACCTCAAACGCGTCAATGCATG
AGTGTAACACGAAGTGTCAAACACCCCAGGGATCTATAAACAGCA
ATCTCCCTTTCCAGAATATACACCCAGTCACAATAGGAGAGTGCC
CAAAATATGTCAGGAGTACCAAATTGAGGATGGTTACAGGACTAA
GAAACATCCCATCCATTCAATACAGAGGTCTATTTGGAGCCATTG
CTGGTTTTATTGAGGGGGGATGGACTGGAATGATAGATGGATGGT
ATGGTTATCATCATCAGAATGAACAGGGATCAGGCTATGCAGCGG
ATCAAAAAAGCACACAAAATGCCATTAACGGGATTACAAACAAGG
TGAACTCTGTTATCGAGAAAATGAACACTCAATTCACAGCTGTGG
GTAAAGAATTCAACAACTTAGAAAAAAGGATGGAAAATTTAAATA
AAAAAGTTGATGATGGGTTTCTGGACATTTGGACATATAATGCAG
AATTGTTAGTTCTACTGGAAAATGAAAGAACTTTGGATTTCCATG
ACTTAAATGTGAAGAATCTGTACGAGAAAGTAAAAAGCCAATTAA
AGAATAATGCCAAAGAAATCGGAAATGGGTGTTTTGAGTTCTACC
ACAAGTGTGACAATGAATGCATGGAAAGTGTAAGAAATGGGACTT
ATGATTATCCAAAATATTCAGAAGAATCAAAGTTGAACAGGGAAA
AGATAGATGGAGTGAAATTGGAATCAATGGGGGTGTATCAGATTC
TGGCGATCTACTCAACTGTCGCCAGTTCACTGGTGCTTTTGGTCT
CCCTGGGGGCAATCAGTTTCTGGATGTGTTCTAATGGGTCTTTGC
AGTGCAGAATATGCATCTGAGATTAGGATTTCAGAAATATAAGGA
AAAACACCCTTGTTTCTACT;

and/or
a wild-type NP gene from an H1N1 virus, having the nucleic acid sequence of:

(SEQ ID NO: 5)
AGCAAAAGCAGGGTAGATAATCACTCACAGAGTGACATCGAAATC
ATGGCGACCAAAGGCACCAAACGATCTTACGAACAGATGGAGACT
GATGGAGAACGCCAGAATGCCACTGAAATCAGAGCATCTGTCGGA
AAAATGATTGATGGAATTGGACGATTCTACATCCAAATGTGCACC
GAACTTAAACTCAGTGATTATGAGGGACGGCTGATTCAGAACAGC
TTAACAATAGAGAGAATGGTGCTCTCTGCTTTTGACGAGAGGAGG
AATAAATATCTAGAAGAACATCCCAGTGCGGGGAAAGATCCTAAG
AAAACTGGAGGACCTATATACAGGAGAGTAGATGGAAAGTGGAGG
AGAGAACTCATCCTTTATGACAAAGAAGAAATAAGACGAATCTGG
CGCCAAGCTAATAATGGTGACGATGCAACGGCTGGTCTGACTCAC
ATGATGATCTGGCACTCCAATTTGAATGATGCAACTTACCAGAGG
ACAAGAGCTCTTGTTCGCACAGGAATGGATCCCAGGATGTGCTCA
CTGATGCAGGGTTCAACCCTCCCTAGGAGGTCTGGGGCCGCAGGT
GCTGCAGTCAAAGGAGTTGGAACAATGGTGATGGAATTGATCAGA
ATGATCAAACGTGGGATCAATGATCGGAACTTCTGGAGGGGTGAG
AATGGACGGAGAACAAGGATTGCTTATGAAAGAATGTGCAACATT
CTCAAAGGGAAATTTCAAACAGCTGCACAAAGAACAATGGTGGAT
CAAGTGAGAGAGAGCCGGAATCCAGGAAATGCTGAGTTCGAAGAT
CTCATCTTTTTAGCACGGTCTGCACTCATATTGAGAGGGTCAGTT
GCTCACAAGTCCTGCCTGCCTGCCTGTGTGTATGGATCTGCCGTA
GCCAGTGGATACGACTTTGAAAGAGAGGGATACTCTCTAGTCGGA
ATAGACCCTTTCAGACTGCTTCAAAACAGCCAAGTATACAGCCTA
ATCAGACCAAATGAGAATCCAGCACACAAGAGTCAACTGGTGTGG
ATGGCATGCCATTCTGCTGCATTTGAAGATCTAAGAGTATCAAGC
TTCATCAGAGGGACGAAAGTGGTCCCAAGAGGGAAGCTTTCCACT
AGAGGAGTTCAAATTGCTTCCAATGAAAACATGGAGACTATGGAA
TCAAGTACCCTTGAACTGAGAAGCAGATACTGGGCCATAAGGACC
AGAAGTGGAGGGAACACCAATCAACAGAGGGCTTCCTCGGGCCAA
ATCAGCATACAACCTACGTTCTCAGTACAGAGAAATCTCCCTTTT
GACAGACCAACCATTATGGCAGCATTCACTGGGAATACAGAGGGG
AGAACATCTGACATGAGAACCGAAATCATAAGGCTGATGGAAAGT
GCAAGACCAGAAGATGTGTCTTTCCAGGGGGGGGGAGTCTTCGAG
CTCTCGGACGAAAAGGCAACGAGCCCGATCGTGCCCTCCTTTGAC
ATGAGTAATGAAGGATCTTATTTCTTCGGAGACAATGCAGAGGAG
TACGACAATTAAAGAAAAATACCCTTGTTTCTACT;

and/or
a wild-type NA gene from an H1N1 virus, having the nucleic acid sequence of:

(SEQ ID NO: 6)
AGCGAAAGCAGGAGTTTAAATGAATCCAAACCAGAAAATAATAAC
CATTGGGTCAATCTGTATGGTAGTCGGAATAATTAGCCTAATATT
GCAAATAGGAAATATAATCTCAATATGGATTAGCCATTCAATTCA
AACCGGAAATCAAAACCATACTGGAATATGCAACCAAGGCAGCAT
TACCTATAAAGTTGTTGCTGGGCAGGACTCAACTTCAGTGATATT
AACCGGCAATTCATCTCTTTGTCCCATCCGTGGGTGGGCTATACA
CAGCAAAGACAATGGCATAAGAATTGGTTCCAAAGGAGACGTTTT
TGTCATAAGAGAGCCTTTTATTTCATGTTCTCACTTGGAATGCAG
GACCTTTTTTCTGACTCAAGGCGCCTTACTGAATGACAAGCATTC
AAGGGGGACCTTTAAGGACAGAAGCCCTTATAGGGCCTTAATGAG
CTGCCCTGTCGGTGAAGCTCCGTCCCCGTACAATTCAAGGTTTGA
ATCGGTTGCTTGGTCAGCAAGTGCATGTCATGATGGAATGGGCTG
GCTAACAATCGGAATTTCTGGTCCAGATGATGGAGCAGTGGCTGT
ATTAAAATACAACCGCATAATAACTGAAACCATAAAAAGTTGGAG
GAAGAATATATTGAGAACACAAGAGTCTGAATGTACCTGTGTAAA
TGGTTCATGTTTTACCATAATGACCGATGGCCCAAGTGATGGGCT
GGCCTCGTACAAAATTTTCAAGATCGAGAAGGGGAAGGTTACTAA
ATCGATAGAGTTGAATGCACCTAATTCTCACTACGAGGAATGTTC
CTGTTACCCTGATACCGGCAAAGTGATGTGTGTGTGCAGAGACAA
TTGGCACGGTTCGAACCGACCATGGGTGTCCTTCGACCAAAACCT
AGATTATAAAATAGGATACATCTGCAGTGGGGTTTTCGGTGACAA
CCCGCGTCCCAAAGATGGAACAGGCAGCTGTGGCCCAGTGTCTGC
TGATGGAGCAAACGGAGTAAAGGGATTTTCATATAAGTATGGCAA
TGGTGTTTGGATAGGAAGGACTAAAAGTGACAGTTCCAGACATGG
GTTTGAGATGATTTGGGATCCTAATGGATGGACAGAGACTGATAG
TAGGTTCTCTATGAGACAAGATGTTGTGGCAATAACTAATCGGTC
AGGGTACAGCGGAAGTTTCGTTCAACATCCTGAGCTAACAGGGCT
AGACTGTATGAGGCCTTGCTTCTGGGTTGAATTAATCAGGGGGCT
ACCTGAGGAGGACGCAATCTGGACTAGTGGGAGCATCATTTCTTT
TTGTGGTGTGAATAGTGATACTGTAGATTGGTCTTGGCCAGACGG
TGCTGAGTTGCCGTTCACCATTGACAAGTAGTTTGTTCAAAAAAC
TCCTTGTTTCTACT;

and/or
a wild-type M gene from an H1N1 virus, having the nucleic acid sequence of:

(SEQ ID NO: 7)
AGCAAAAGCAGGTAGATATTGAAAGATGAGTCTTCTAACCGAGGT
CGAAACGTACGTTCTCTCTATCGTCCCGTCAGGCCCCCTCAAAGC
CGAGATCGCACAGAGACTTGAAGATGTCTTTGCAGGGAAGAACAC
CGATCTTGAGGTTCTCATGGAATGGCTAAAGACAAGACCAATCCT
GTCACCTCTGACTAAGGGGATTTTAGGATTTGTGTTCACGCTCAC
CGTGCCCAGTGAGCGGGGACTGCAGCGTAGACGCTTTGTCCAAAA
TGCTCTTAATGGGAACGGAGATCCAAATAACATGGACAAAGCAGT
TAAACTGTATAGGAAGCTTAAGAGGGAGATAACATTCCATGGGGC
CAAAGAAATAGCACTCAGTTATTCTGCTGGTGCACTTGCCAGTTG
TATGGGCCTCATATACAACAGGATGGGGGCTGTGACCACTGAAGT
GGCATTTGGCCTGGTATGCGCAACCTGTGAACAGATTGCTGACTC
CCAGCATCGGTCTCATAGGCAAATGGTGACAACAACCAATCCACT
AATCAGACATGAGAACAGAATGGTTCTAGCCAGCACTACAGCTAA
GGCTATGGAGCAAATGGCTGGATCGAGTGAGCAAGCAGCAGAGGC
CATGGATATTGCTAGTCAGGCCAGGCAAATGGTGCAGGCGATGAG
AACCGTTGGGACTCATCCTAGCTCCAGTGCTGGTCTAAAAGATGA
TCTTCTTGAAAATTTGCAGGCCTATCAGAAACGAATGGGGGTGCA
GATGCAACGATTCAAGTGATCCTCTCGTCATTGCAGCAAATATCA
TTGGAATCTTGCACTTGATATTGTGGATTCTTGATCGTCTTTTTT
TCAAATGCATTTATCGTCGCTTTAAATACGGTTTGAAAAGAGGGC
CTTCTACGGAAGGAGTGCCAGAGTCTATGAGGGAAGAATATCGAA
AGGAACAGCAGAATGCTGTGGATGTTGACGATGGTCATTTTGTCA
ACATAGAGCTGGAGTAAAAAACTACCTTGTTTCTACT;

and/or
a wild-type NS gene from an H1N1 virus, having the nucleic acid sequence of:

(SEQ ID NO: 8)
AGCAAAAGCAGGGTGACAAAGACATAATGGATCCAAACACTGTGT
CAAGCTTTCAGGTAGATTGCTTTCTTTGGCATGTCCGCAAAAGAG
TTGCAGACCAAGAACTAGGTGATGCCCCATTCCTTGATCGGCTTC
GCCGAGATCAGAAGTCCCTAAGAGGAAGAGGCAGCACTCTTGGTC
TGGACATCGAAACAGCCACCCGTGCTGGAAAGCAAATAGTGGAGC
GGATTCTGAAGGAAGAATCTGATGAGGCACTCAAAATGACCATGG
CCTCTGTACCTGCATCGCGCTACCTAACTGACATGACTCTTGAGG
AAATGTCAAGGCACTGGTTCATGCTCATGCCCAAGCAGAAAGTGG
CAGGCCCTCTTTGTATCAGAATGGACCAGGCGATCATGGATAAGA
ACATCATACTGAAAGCGAACTTCAGTGTGATTTTTGACCGGCTGG
AGACTCTAATATTACTAAGGGCCTTCACCGAAGAGGGGACAATTG
TTGGCGAAATTTCACCACTGCCCTCTCTTCCAGGACATACTGATG
AGGATGTCAAAAATGCAGTTGGGGTCCTCATCGGAGGACTTGAAT
GGAATAATAACACAGTTCGAGTCTCTGAAACTCTACAGAGATTCG
CTTGGAGAAGCAGTAATGAGAATGGGAGACCTCCACTCACTCCAA
AACAGAAACGGAAAATGGCGGGAACAATTAGGTCAGAAGTTTGAA
GAAATAAGATGGTTGATTGAAGAAGTGAGACACAGACTGAAGATA
ACAGAGAATAGTTTTGAGCAAATAACATTTATGCAAGCCTTACAA
CTATTGCTTGAAGTGGAGCAAGAGATAAGAACTTTCTCGTTTCAG
CTTATTTAATAATAAAAAACACCCTTGTTTCTACT.

Iii. Defective-Interfering (DI) Particles

In some forms the live, non-replicating or replication-impaired influenza virus is, or is derived from a defective-interfering (DI) particle produced by a wild-type influenza virus. For example, in some forms the modified, intact, replication-deficient virus includes one or more truncated or otherwise mutated genes obtained from a defective-interfering (DI) particle.

Defective-interfering (DI) particles are defective viruses that contain internal truncations in viral genome. Despite being non-replicative due to the loss of essential viral gene expression, DI particles are able to enter cells in the same way as a standard virus does (Fazekas De St. Groth, et al., Nature 173, 637-638 (1954); Huang and Baltimore, Nature 226, 325-327 (1970)). Therefore, in some forms, live, non-replicating or replication-impaired influenza viruses include DI particles that are infectious but completely non-replicative in primary human cells.

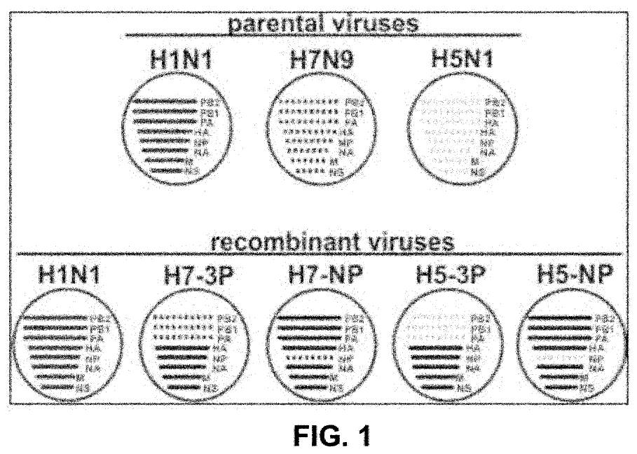

The presence of DI particles was only sporadically reported on certain laboratory strain viruses, and the amount of DI particles is typically small when compared with the prototypic virus. However, abundant presence of defective interfering genomes in influenza A/H7N9 virus-infected patient samples has been observed (Lui, et al., Emerg Microbes Infect 8, 662-674 (2019)). Therefore, in some forms, the DI particle is derived from an influenza virus having an increased or high level of polymerase activity. In some forms, defective genomes and DI particles are prepared, isolated and assessed in a panel of chimeric A/H1N1 viruses, for example, as described in the Examples and FIG. 1.

### B. Adjuvants

In some forms, the compositions including replication-deficient influenza viruses also include one or more adjuvants. Useful adjuvants but are not limited to, one or more set forth below.

In some forms, the compositions include one or more mineral containing adjuvants (MCA). Mineral containing adjuvant compositions include mineral salts, such as aluminum salts and calcium salts. Exemplary mineral salts include hydroxides (e.g., oxyhydroxides), phosphates (e.g., hydroxyphosphates, orthophosphates), sulfates, and the like or mixtures of different mineral compounds (e.g., a mixture of a phosphate and a hydroxide adjuvant, optionally with an excess of the phosphate), with the compounds taking any suitable form (e.g., gel, crystalline, amorphous, and the like), and with adsorption to the salt(s) being preferred. The mineral containing compositions can also be formulated as a particle of metal salt (See, e.g., International Publication No. WO/0023105 incorporated herein by reference in entirety.). Aluminum salts can be included in compositions of the invention such that the dose of Al³⁺ is between 0.2 mg and 1.0 mg, inclusive, per dose.

In some forms, the compositions include one or more Oil-Emulsion Adjuvants (OEA). Oil-emulsion adjuvants suitable for use as adjuvants in the invention can include squalene-water emulsions, such as MF59 (5% Squalene, 0.5% Tween 80, and 0.5% Span 85, formulated into submicron particles using a microfluidizer). See, e.g., International Publication No. WO90/14837; and Podda, Vaccine 19:2673-2680, 2001. Additional adjuvants for use in the compositions are submicron oil-in-water emulsions. Examples of submicron oil-in-water emulsions for use herein include squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-s- -n-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entirety. MF59 can contain 4-5% w/v Squalene (e.g., 4.3%), 0.25-0.5% w/v Tween 80, and 0.5% w/v Span 85 and optionally contains various amounts of MTP-PE, formulated into submicron particles using a microfluidizer such as Model 110Y microfluidizer (Microfluidics, Newton, Mass.). For example, MTP-PE can be present in an amount of about 0-500 μg/dose, or 0-250 μg/dose, or 0-100 μg/dose. Submicron oil-in-water emulsions, methods of making the same and immuno-stimulating agents, such as muramyl peptides, for use in the compositions, are described in detail in International Publication No. WO90/14837 and U.S. Pat. Nos. 6,299,884 and 6,451,325 which are incorporated herein by reference in their entirety.

In some forms, complete Freund's adjuvant (CFA) and/or incomplete Freund's adjuvant (IFA) are also be used as adjuvants.

In some forms, the compositions include one or more saponin adjuvants. Saponin Adjuvant Formulations can also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations can include purified formulations, such as QS21, as well as lipid formulations, such as Immunostimulating Complexes (ISCOMs; see below). Saponin compositions have been purified using High Performance Thin Layer Chromatography (HPLC) and Reversed Phase High Performance Liquid Chromatography (RP-HPLC). Specific purified fractions using these techniques have been identified, including QS7, QS17, QS18, QS21, QH-A, QH-B and QH-C. A method of production of QS21 is disclosed in U.S. Pat. No. 5,057,540. Saponin formulations can also include a sterol, such as cholesterol (see WO96/33739). Combinations of saponins and cholesterols can be used to form unique particles called ISCOMs. ISCOMs typically also include a phospholipid such as phosphatidylethanolamine or phosphatidylcholine. Any known saponin can be used in ISCOMs. For example, an ISCOM can include one or more of Quil A, QHA and QHC. ISCOMs are described in EP0109942, WO96/11711, and WO96/33739 which are incorporated herein by reference in their entirety. Optionally, the ISCOMS can be devoid of additional detergent. See WO00/07621. A description of the development of saponin based adjuvants can be found at Barr, et al., “ISCOMs and other saponin based adjuvants,” Advanced Drug Delivery Reviews 32:247-27, 1998. See also Sjolander, et al., “Uptake and adjuvant activity of orally delivered saponin and ISCOM vaccines,” Advanced Drug Delivery Reviews 32:321-338, 1998.

In some forms, the compositions include one or more Bacterial or Microbial Derivatives as adjuvants. Bacterial or Microbial Derivatives useful as adjuvants include: (i) Non-Toxic Derivatives of Enterobacterial Lipopolysaccharide (LPS); (ii) lipid derivatives, (iii) immunostimulatory oligonucleotides and ADP-Ribosylating Toxins and Detoxified Derivatives Thereof, (iv) ADP-Ribosylating Toxins and Detoxified Derivatives Thereof. Examples of Non-Toxic Derivatives of LPS Monophosphoryl lipid A (MPL) and 3-O-deacylated MPL (3 dMPL). 3 dMPL is a mixture of 3 De-O-acylated monophosphoryl lipid A with 4, 5 or 6 acylated chains. An example of a “small particle” form of 3 De-O-acylated monophosphoryl lipid A is disclosed in EP 0 689 454. Such “small particles” of 3 dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g., RC-529 (Johnson et al., Bioorg Med Chem Lett, 9:2273-2278, 1999). Examples of lipid A derivatives can include derivatives of lipid A from Escherichia coli such as OM-174. OM-174 is described for example in Meraldi et al., Vaccine 21:2485-2491, 2003; and Pajak, et al., Vaccine 21:836-842, 2003. Examples of immunostimulatory oligonucleotides nucleotide sequences containing a CpG motif (a sequence containing an unmethylated cytosine followed by guanosine and linked by a phosphate bond). Bacterial double stranded RNA or oligonucleotides containing palindromic or poly (dG) sequences have also been shown to be immunostimulatory.

The CpG's can include nucleotide modifications/analogs such as phosphorothioate modifications and can be double-stranded or single-stranded. Optionally, the guanosine can be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles,” Nucleic Acids Research 31:2393-2400, 2003; WO02/26757 and WO99/62923 for examples of analog substitutions. The adjuvant effect of CpG oligonucleotides is further discussed in Krieg, Nature Medicine (2003) 9 (7): 831-835; McCluskie, et al., FEMS Immunology and Medical Microbiology (2002) 32:179-185; WO98/40100; U.S. Pat. Nos. 6,207,646; 6,239,116 and 6,429,199. The CpG sequence can be directed to Toll-like receptor (TLR9), such as the motif GTCGTT or TTCGTT. See Kandimalla, et al., “Toll-like receptor 9: modulation of recognition and cytokine induction by novel synthetic CpG DNAs,” Biochemical Society Transactions (2003) 31 (part 3): 654-658. The CpG sequence can be specific for inducing a Th1 immune response, such as a CpG-A ODN, or it can be more specific for inducing a B cell response, such a CpG-B ODN. CpG-A and CpG-B ODNs are discussed in Blackwell, et al., J. Immunol. 170:4061-4068, 2003; Krieg, TRENDS in Immunology 23:64-65, 2002, and WO01/95935. In some aspects, the CpG oligonucleotide can be constructed so that the 5′ end is accessible for receptor recognition. Optionally, two CpG oligonucleotide sequences can be attached at their 3′ ends to form “immunomers.” See, for example, Kandimalla, et al., BBRC 306:948-95, 2003; Kandimalla, et al., Biochemical Society Transactions 31:664-658, 2003; Bhagat et al., “BBRC 300:853-861, 2003, and WO03/035836. Bacterial ADP-ribosylating toxins and detoxified derivatives thereof can be used as adjuvants in the invention. For example, the toxin can be derived from E. coli (i.e., E. coli heat labile enterotoxin (LT)), cholera (CT), or pertussis (PTX). The use of detoxified ADP-ribosylating toxins as mucosal adjuvants is described in WO95/17211 and as parenteral adjuvants in WO98/42375. In some aspects, the adjuvant can be a detoxified LT mutant such as LT-K63, LT-R72, and LTR192G. The use of ADP-ribosylating toxins and detoxified derivatives thereof, particularly LT-K63 and LT-R72, as adjuvants can be found in the following references, each of which is specifically incorporated by reference herein in their entirety: Beignon, et al., Infection and Immunity 70:3012-3019, 2002; Pizza, et al., Vaccine 19:2534-2541, 2001; Pizza, et al., Int. J. Med. Microbiol 290:455-461, 2003; Scharton-Kersten et al., Infection and Immunity 68:5306-5313, 2000; Ryan et al., Infection and Immunity 67:6270-6280, 2003; Partidos et al., Immunol. Lett. 67:09-216, 1999; Peppoloni et al., Vaccines 2:285-293, 2003; and Pine et al., J. Control Release 85:263-270, 2002.

In some forms, Bioadhesives and mucoadhesives are used as adjuvants. Suitable bioadhesives can include esterified hyaluronic acid microspheres (Singh et al., J. Cont. Rel. 70:267-276, 2001) or mucoadhesives such as cross-linked derivatives of poly(acrylic acid), polyvinyl alcohol, polyvinyl pyrollidone, polysaccharides and carboxymethylcellulose. Chitosan and derivatives thereof can also be used as adjuvants in the invention disclosed for example in WO99/27960.

Additional adjuvants include polyoxyethylene ethers and polyoxyethylene esters, for example, as described in WO99/52549. Such formulations can further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). In some aspects, polyoxyethylene ethers can include: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, or polyoxyethylene-23-lauryl ether.

PCPP formulations for use as adjuvants are described, for example, in Andrianov et al., Biomaterials 19:109-115, 1998.1998. Examples of muramyl peptides suitable for use as adjuvants in the invention can include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s- -n-glycero-3-hydroxyphosphoryloxy)-ethylamine MTP-PE). Examples of imidazoquinolone compounds suitable for use as adjuvants in the invention can include Imiquimod and its homologues, described further in Stanley, “Imiquimod and the imidazoquinolones: mechanism of action and therapeutic potential” Clin Exp Dermatol 27:571-577, 2002 and Jones, “Resiquimod 3M,” Curr Opin Investig Drugs 4:214-218, 2003.

Adjuvant Combinations: The adjuvants are used in come preferred embodiments as combinations. For example, adjuvant compositions can include: a saponin and an oil-in-water emulsion (WO99/11241); a saponin (e.g., QS21)+a non-toxic LPS derivative (e.g., 3 dMPL) (see WO94/00153); a saponin (e.g., QS21)+a non-toxic LPS derivative (e.g., 3 dMPL)+a cholesterol; a saponin (e.g., QS21)+3 dMPL+IL-12 (optionally+a sterol) (WO98/57659); combinations of 3 dMPL with, for example, QS21 and/or oil-in-water emulsions (See European patent applications 0835318, 0735898 and 0761231); SAF, containing 10% Squalane, 0.4% Tween 80, 5% pluronic-block polymer L121, and thr-MDP, either microfluidized into a submicron emulsion or vortexed to generate a larger particle size emulsion. Ribi adjuvant system (RAS), (Ribi Immunochem) containing 2% Squalene, 0.2% Tween 80, and one or more bacterial cell wall components including monophosphorylipid A (MPL), trehalose dimycolate (TDM), and cell wall skeleton (CWS), preferably MPL+CWS (Detox); and one or more mineral salts (such as an aluminum salt)+a non-toxic derivative of LPS (such as 3 dPML).

Aluminum salts and MF59 are examples of adjuvants for use with injectable influenza vaccines. Bacterial toxins and bioadhesives are examples of adjuvants for use with mucosally-delivered vaccines, such as nasal vaccines. All adjuvants noted above and others as generally known in the art to one of ordinary skill can be formulated for intradermal administration using techniques well known in the art.

### C. Immunostimulatory Molecules

In certain forms the compositions including non-replicating influenza viruses include one or more immunostimulatory molecules, such as cytokines. For example, in some forms, cytokines can be administered separately, locally, or systemically to the host. It may be desirable to administer a substantially pure preparation of the immunomodulator to boost vaccine efficacy.

Therefore, in some forms, the compositions include one or more costimulatory molecules including, but not limited to, B7-1, B7-2, ICAM-1, CD40, CD40L, LFA-3, CD72, OX40L (with or without OX40). Examples of cytokines and growth factors include, but are not limited to, granulocyte macrophage-colony stimulating factor (GM-CSF), granulocyte-colony stimulating factor (G-CSF), macrophage-colony stimulating factor (M-CSF), tumor necrosis factors (TNFα and TNFβ), transforming growth factors (TGFα and TGFβ), insulin-like growth factors (IGF-I and IGF-II), growth hormone, interleukins 1 to 15 (IL-1 to IL-15), interferons α, β, γ (IFN-α IFN-β and IFN-γ), brain-derived neurotrophic factor, neurotrophins 3 and 4, hepatocyte growth factor, erythropoictin, EGF-like mitogens, TGF-like growth factors, PDGF-like growth factors, melanocyte growth factor, mammary-derived growth factor 1, prostate growth factors, cartilage-derived growth factor, chondrocyte growth factor, bone-derived growth factor, osteosarcoma-derived growth factor, glial growth-promoting factor, colostrum basic growth factor, endothelial cell growth factor, tumor angiogenesis factor, hematopoietic stem cell growth factor, B-cell stimulating factor 2, B-cell differentiation factor, leukemia-derived growth factor, myelomonocytic growth factor, macrophage-derived growth factor, macrophage-activating factor, erythroid-potentiating activity, keratinocyte growth factor, ciliary neurotrophic growth factor, Schwann cell-derived growth factor, vaccinia virus growth factor, bombyxin, neu differentiation factor, v-Sis, glial growth factor/acetylcholine receptor-inducing activity, transferrin, bombesin and bombesin-like peptides, angiotensin II, endothelin, atrial natriuretic factor (ANF) and ANF-like peptides, vasoactive intestinal peptide, RANTES, Bradykinin and related growth factors.

In some forms, the co-stimulatory molecule, growth factor, adjuvant or cytokine is IL-1, IL-2, IL-4, IL-7, IL1-9, IL-12, IL-15, IL-18, IL-23, IL-27, IL-31, IL-33, B7-1, B7-2, B7-H3, LFA-3, B7-H3, CD40, CD40L, ICOS-ligand, OX-40L, 4-1BBL, GM-CSF, SCF, FGF, Flt3-ligand, CCR4, QS-7, QS-17, QS-21, CpG oligonucleotides, ST-246, AS-04, LT R192G mutant, Montanide ISA 720, heat shock proteins, synthetic mycobacterial cordfactor (CAF01), Lipid A mimetics, Salmonella enterica serovar Typhimurium flagellin (FliC), Montanide 720, Levamisole (LMS), Imiquimod, Diphtheria Toxin, IMP321, AS02A, AS01B, AS15-SB, Alhydrogel, Montanide ISA, Aluminum hydroxide, MF59, ISCOMATRIX, MLPA, MPL and other TLR-4 ligands, MDP and other TLR-2 ligands, CpG and TLR9 ligands, imiquimod and other TLR7 ligands, resiquimod and TLR8 ligands, AS02A, AS01B, Heat Liable Toxin LTK63 and LT-R192G.

In some forms, the compositions include OX40. OX40 is used as an immunostimulatory a primary co-stimulator of T cells that have encountered antigen, rather than naive T cells, and promotes T-cell expansion after T cell tolerance is induced. (Bansal-Pakal et al., Nature Med. 7:907-12 (2001)). OX40L plays a role during T cell activation by a) sustaining the long-term proliferation of CD4⁺ and CD8⁺ T cells, b) enhancing the production of Th1 cytokines such as IL-2, IGN-γ, and TNF-α from both CD4⁺ and CD8⁺ T cells without changing IL-4 expression, c) protecting T cells from apoptosis. In certain embodiments, the combination of B7-1, ICAM-1, LFA-3, and OX40L enhances initial activation and then further potentiates sustained activation of naive and effector T cells.

### D. Pharmaceutical Excipients

In some forms, compositions including non-replicating influenza viruses include one or more other pharmaceutically acceptable carriers, including any suitable diluent or excipient.

For intradermal injection, or injection at the site of affliction, the compositions including non-replicating influenza viruses are typically formulated in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants, and/or other additives can be included, as required.

Administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

Preferably, the pharmaceutically acceptable carrier does not itself induce a physiological response, e.g., an immune response, nor result in any adverse or undesired side effects and/or does not result in undue toxicity. Pharmaceutically acceptable carriers include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition).

### III. Methods of Making Replication-Deficient Influenza Vaccine Compositions

### A. Protocol to Engineer Non-Replicating Influenza Viruses

Protocols to engineer a non-replicating chimeric influenza virus is provided for in the examples. Typically, the protocol includes the steps of:
(a) Construction of a chimeric virus using a target backbone influenza virus (e.g., a laboratory strain), with the three polymerase subunits or nucleoprotein from a strain with a highly-active polymerase (e.g., A/H7N9 or A/H5N1);
(b) Identification and purification of defective-interfering (DI) particles, (chimeric backbone/H7 or H5 viruses) possessing either H5N1 or H7N9 polymerases or nucleoprotein;
(c) Determination of the sequence of the most abundant defective (DI) particle viral genome, for example, using third-generation Single Molecule Real-Time (SMRT) long-read sequencing technology (as described in Lui, et al., Emerg Microbes Infect 8, 662-674 (2019), the content of which is incorporated herein by reference in its entirety) to identify the sequence of the most abundant defective genome; and
(d) large-scale production of high-titer non-replicating influenza viruses.

In an exemplary method, a modified reverse genetics system is used to produce high-titer non-replicating influenza viruses prepared according to the steps (a)-(d), without contamination with standard virus. In some forms, a stable cell line is used, for example, MDCK or 293-T cells trans-complemented with a PB2 or NP transgene are established.

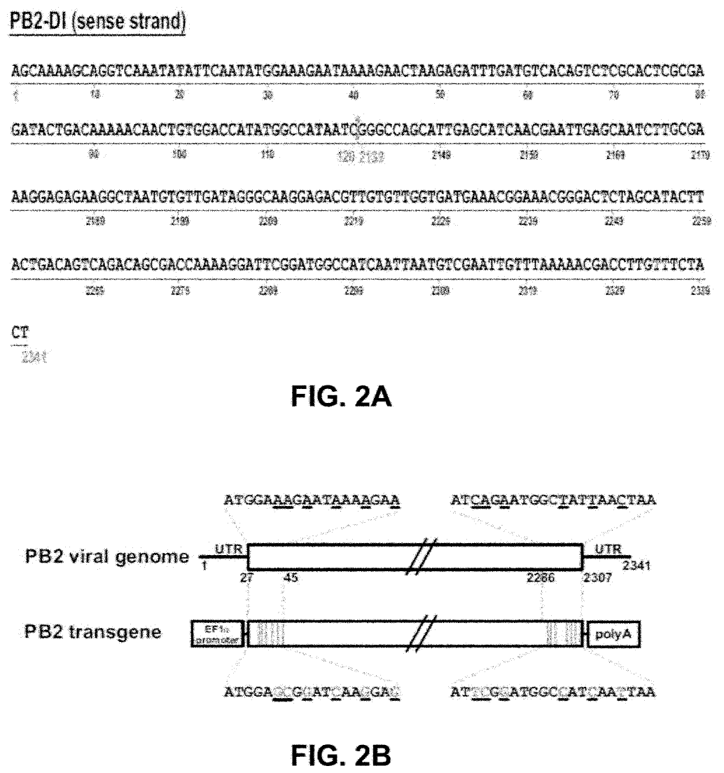

In some forms, the methods introduce additional mutations into the PB2 or NP mutant genes. For example, in some forms, between one and one hundred mutations next to the start and stop codons are introduced into the PB2 or NP transgene to minimize the chance of its recombination with the viral genome during chimeric viral production (See, Examples and FIGS. 2A and B).

In some forms, the chimeric non-replicating virus is produced by transfecting the reverse genetic plasmids into a suitable cell line, for example, with the functional-PB2 plasmid replaced with a non-functional PB2 plasmid. An exemplary non-functional PB2 is a PB2 that is a truncated. An exemplary truncated PB2 is the truncated non-functional PB2 having SEQ ID NO: 1, or a derivative thereof.

In some forms, the methods produce the replication-deficient virus in cell-lines and purify the recombinant replication-deficient virus. In an exemplary method, replication-deficient virus is produced in the cell-lines, and isolated by purification. Viruses can be purified by any method known in the art. For example, in some forms, virus produced is purified by plaque-purification. In other forms, purification is by sucrose-cushioned ultracentrifugation. Typically, the non-replicating influenza viruses produced and purified according to the methods have a size similar to wildtype virions, which were reported to be in spherical or elliptical shapes with a diameter of from about 80 nm to about 120 nm, inclusive.

### B. Culturing of Non-Replicating Influenza Viruses

In some forms the methods further propagate the virus in a suitable cell-line. A suitable cell line includes MDCK-PB2 cells. In an exemplary form, the methods pool and concentrate the virus to a titer of between 1×10³ and 2.5×10⁹ PFU/ml, inclusive. The recombinant virus is completely replication-defective as evidenced by the absence of plaques in parental host cell lines, but present in MDCK-PB2 cells. The non-replicating influenza viruses produced and purified according to the methods remain infectious and stable over serial passaging. In preferred forms, the methods do not produce replicating viruses that have reverted to full-length PB2, nor do the methods produce viruses having further truncation of PB2. Most viral vaccines such as attenuated or recombinant viruses are manufactured from cell culture systems. The cells used for virus/vaccine production may be cell lines, i.e. cells that grow continuously in vitro, either as single-cell suspension culture in bioreactors or as a monolayer on a cell-support surface of tissue culture flasks or roller-bottles. In some forms, primary animal cells are used for the manufacture of vaccines. For example, in some forms, influenza viruses are amplified in cell cultures of primary or secondary chicken embryo fibroblasts (CEF), or African green monkey kidney (Vero) cells. In some forms, influenza viruses are amplified in cell cultures of primary or secondary MDCK cells, or 293 cells.

In some forms, CEK cells are obtained from embryos of chicken eggs that are incubated for 10 to 12 days. The cells of the embryos are then dissociated and purified. These primary CEF cells can either be used directly or after one further cell passage as secondary CEF cells. Subsequently, the primary or secondary CEF cells are infected with the replication-deficient influenza viruses. In some forms, the replication-deficient influenza viruses are not propagated on human cells since there is a concern that the viruses might become replication competent in cells of human origin. Viruses that have regained the ability to replicate in human cells represent a health risk if administered to humans, in particular if the individuals are immune compromised. For this reason, in some forms, the replication-deficient influenza viruses are propagated in CEF cells if intended for human use.

Once a recombinant replication-deficient virus has been produced and purified, a variety of methods well known in the art can be used to characterize the recombinant virus. These methods include, for example, black plaque assay (an in situ enzyme immunoassay performed on viral plaques), Western blot analysis, enzyme immunoassay (EIA), radioimmunoprecipitation (RIPA), or functional assay such as CTL assay.

An exemplary method of making an intact, replication-deficient virus having an influenza virus genome with one or more mutations in one or more genes selected from viral RNA polymerase PB1, viral RNA polymerase PB2, viral RNA polymerase PA, and nucleoprotein (NP), includes the steps of
(a) introducing into a first cell genes encoding 4-7 segments of a wild-type virus; and
(b) introducing into a second cell gene(s) encoding a mutant PB1, PB2, PA, and/or NP, and
(c) co-culturing of the first and second cells; and
(d) isolating the intact, replication-deficient virus.

In some forms, the first and/or second cell is selected from a primary or secondary chicken embryo fibroblasts (CEF), an African green monkey kidney (Vero) cell, a MDCK cell and a 293FT cell.

In some forms, the mutant PB1, PB2, PA, and/or NP gene(s) is introduced into the cell by lentivirus transduction, for example, within an expression vector which is driven by an RNA polymerase I promoter.

In some forms, the isolating in step (d) includes one or more steps of purification by filtration, for example, by passage through one or more 0.45 μm filter.

In some forms, the isolating in step (d) includes purification by ultracentrifugation, such as by sucrose-cushioned ultracentrifugation at 28,000 rpm for 4 hours.

### IV. Methods of Treatment

Methods for inducing or stimulating a protective, broadly cross-reactive T cell mediated immune response to influenza viruses in epidermal tissues have been established. Typically, the methods deliver compositions including intact, non-replicating or replication impaired, infectious influenza viruses via intradermal administration. The vaccine composition is typically administered in an amount sufficient to stimulate a protective immune response in the recipient.

Because the replication-deficient influenza viruses have the same or substantially the same external structures as the wild-type backbone viruses on which they are based, they raise an immune response in a recipient subject against one or more of the influenza antigens present on the wild-type virus, including one or more of the NA, HA, and M genes.

Criteria that have been identified for development of next-generation influenza vaccines include: i) optimal activation of innate immune response; ii) increased dose of antigen delivered; and iii) intradermal route of administration.

As demonstrated in the Examples, it has been established that rationally designed and productively generated vaccine prototypes (named IntraDermal Influenza Vaccine (IDIV)), including a single-round infectious influenza virus, induce optimal innate immune responses by the activation of skin-associated immune cells such as dendritic cells and macrophages, followed by monocyte infiltration and differentiation into monocyte-derived dendritic cells (MoDC) for antigen capture and presentation. It has been demonstrated that replication-incompetent IDIV activate innate immune responses in human blood-derived macrophages and dendritic cells. Since IDIV is non-replicative, it allows intradermal administration at high dose with minimal concern about adverse inflammatory effect caused by viral replication. High-dose IDIV administration in mice did not trigger adverse effects such as inflammation but induced high level of serum interferons and chemokines within one day post-vaccination. The early elevation of serum type-I/II interferons and chemokines level suggested an optimal innate immune activation, which could be beneficial to the subsequent development of adaptive immune response. As expected, expedited seroconversion and high-titer neutralizing antibody production were observed as early as 7 days post-vaccination. It was also demonstrated that single dose of IDIV, without the application of exogenous adjuvants, could protect mice from lethal influenza A virus infection as early as 7 days post-vaccination. Most importantly, IDIV elicited protection against heterologous H1N1, H5N1 and H7N9 lethal challenge. Collectively, IDIV displayed unprecedented performance, shedding new light on the development of new influenza vaccines and so merits further characterization.

Methods for inducing or stimulating a protective immune response to an influenza virus in a subject include administering to epidermal tissues of the subject a vaccine composition including intact, replication-deficient virus having an influenza virus genome, whereby the virus infects normal human cells, whereby the influenza virus genome includes one or more mutations in one or more genes including viral RNA polymerase PB1, viral RNA polymerase PB2, viral RNA polymerase PA, and nucleoprotein (NP), and whereby the one or more mutations prevents or reduces replication of the virus in normal human cells by at least 90% as compared to the same virus in the absence of the mutation. The methods administer an amount of the vaccine composition effective to induce or stimulate the immune response in the subject. In preferred forms, the vaccine composition is administered to the subject by intradermal injection.

Typically, the methods provide protective immunity to two or more different strains of influenza viruses in the recipient. In some forms, the methods provide protective immunity to one or more H1N1 influenza viruses and one or more H3N2 influenza viruses, and/or one or more H5N1 influenza viruses and/or one or more H7N9 influenza viruses.

In some forms, the methods administer a co-stimulatory molecule, a growth factor, an adjuvant and/or a cytokine, before, at the same time, or after the administration of the replication deficient influenza virus. The co-administered agent can be in the same, or different composition, and can be administered at the same or a distant site. Suitable stimulatory molecules, growth factors, adjuvants and cytokines include IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Flt3-ligand, and CCR4.

In some forms, the methods repeat the step of administering the non-replicating influenza virus composition. The repeated administration can be via intradermal administration, or by any other route, such as intramuscular, intravenous, subcutaneous or enteral administration.

Typically, the second administration is carried out at a time of one, two, three, four, five, six, seven, eight, nine, or ten days or weeks after the first administration.

In preferred forms, the methods provide protective immunity to two or more different influenza viruses. For example, in some forms, the methods provide protective immunity to two, three, four, five, six, seven, eight, nine, ten or more than ten different influenza viruses, following one or more administrations of the same replication-deficient influenza virus.

Typically, the methods induce or stimulate a broadly-cross reactive immune response in the recipient subject against one or more of the influenza antigens present on the wild-type virus, including one or more of the NA, HA, and M gene

### A. Individuals to be Vaccinated

The methods administer intact, replication-deficient influenza viruses to a subject having or at risk of having an influenza infection. Because the compositions are typically administered to a human subject in the form of a vaccine, the subject may be referred to as a recipient or a vaccinee.

A subject having an infection is a subject that has been exposed to an infectious microorganism and has acute or chronic detectable levels of the microorganism in his/her body or has signs and symptoms of the infectious microorganism. Methods of assessing and detecting infections in a subject are known by those of ordinary skill in the art. A subject at risk of having an infection is a subject that may be expected to come in contact with infectious microorganisms. Examples of such subjects are medical workers or those traveling to parts of the world where the incidence of infection is high. In some embodiments, the subject is at an elevated risk of an infection because the subject has one or more risk factors to have an infection. Examples of risk factors to be infected include immunosuppression, immunocompromised, age (advanced or very young), trauma, burns, surgery, and cancer. The degree of risk of infection depends on the multitude and the severity or the magnitude of the risk factors that the subject has. Risk charts and prediction algorithms are available for assessing the risk of an infection in a subject based on the presence and severity of risk factors. In some forms, subjects include the elderly (e.g., >65 years old), or young children (e.g., <5 years old). Other methods of assessing the risk of infection in a subject are known by those of ordinary skill in the art. In some embodiments, the subject who is at an elevated risk of an infection may be an apparently healthy subject. An apparently healthy subject is a subject who has no signs or symptoms of disease.

### B. Administration and Dosages

Typically, the methods administer the compositions of intact, replication-deficient influenza viruses by intradermal injection. Intradermal injections (ID) are injections administered into the dermis, just below the epidermis. The ID injection route has the longest absorption time of all parenteral routes. Direct delivery of a first dose can be accomplished by injection (e.g., intradermal injection). A second or further dose can be delivered by the transdermal route, or by other routes (e.g., subcutaneously, intraperitoneally, intravenously, intramuscularly, or to the interstitial space of a tissue).

In some forms, administration includes two or more vaccinations that are part of an immunization schedule. For example, in some forms, a repeated administration is carried out at a time of one, two, three, four, five, six, seven, eight, nine, or ten days or weeks or months or years after the first administration. In some forms, the non-replicating influenza virus and/or compositions thereof are administered on a dosage schedule, for example, an initial administration of a first dose, with subsequent booster administrations of a second or further dose. In some forms, the first and second or further doses are the same, or different.

It has been established that intradermal administration of non-replicating intact influenza viruses to a subject enhances the efficacy of vaccination and provides a broader and more fully protective immunity in the recipient, as compared with administration of the same amount of the same non-replicating intact influenza viruses to the subject by other routes. Therefore, in some forms the methods induce or stimulate broadly cross reactive immunity to influenza in a subject by administering a smaller dose of the compositions by the intradermal route than would be necessary to achieve the same immunity when administering the same compositions by other routes. For example, in some forms, an immune response to influenza viruses can be generated by administering between about 2-fold to about a 100-fold less pfu (plaque forming units) of the non-replicating influenza virus, when applied by intradermal administration, as compared to conventional injection routes. In an exemplary method, intradermal administration of the same non-replicating influenza virus provides equivalent similar immunogenicity to a dose that is 50%, 100%, 200%, 300%, 400%, 500%, 600%, 750%, or 1000% or more greater when administered by the intramuscular route. In certain forms, a specific immune response to influenza viruses can be generated by administering between about 90-fold, 80-fold, 70-fold, 60-fold, 50-fold, 40-fold, 30-fold, 20-fold, 10-fold, 5-fold less pfu of the non-replicating influenza virus when applied by intradermal administration, compared to conventional injection routes.

In some forms a single deposition of non-replicating influenza virus is required to elicit a long-lasting, potent influenza-specific immune response in the subject. In an exemplary form, the dose of non-replicating influenza virus is from about 1.0×10⁵ PFU to about 2.5×10¹⁰ PFU, inclusive, for example, a high-dose is from about 2×10⁷ PFU to about 2×10⁹ PFU, inclusive, such as 2×10⁸ PFU, and a low-dose is from about 2×10⁵ PFU to about 5×10⁶ PFU, inclusive, such as 2×10⁶ PFU.

In some forms the amount of the non-replicating influenza virus administered is effective to decrease or inhibit the infection, viability, proliferation or a combination thereof of a wild-type influenza virus in the subject compared to an untreated control subject. In some forms, the amount of the non-replicating influenza virus administered is effective to reduce, slow or halt infection, viability, proliferation, or a combination thereof of a wild-type influenza virus, or to reduce disease burden, morbidity or mortality in the recipient, or a combination thereof. In some forms, the amount of the non-replicating influenza virus administered to a subject as a vaccine is effective to alter a measurable biochemical or physiological marker in the subject, as compared to an untreated control subject who has not been administered the same vaccine. In some forms, the result achieved by the administration of non-replicating influenza virus vaccine is effective to increase the antigen-specific antibody concentration in the blood of the recipient, produce a greater amount of antigen-specific T-cells in the recipient, produce a greater amount of antigen-specific B-cells in the recipient, or a combination thereof, as compared to the results achieved by administering conventional split influenza vaccine, or no vaccine to a control subject. For example, the amount of non-replicating influenza virus vaccine administered to a subject can be effective to increase one or more of the level or concentration of influenza-specific antibodies in the blood, the level or concentration of influenza-specific T-cells in the recipient, the level or concentration of influenza-specific B-cells in the recipient, or a combination thereof compared to the level(s) or concentration(s) in the blood prior to treatment, or compared to the level(s) or concentration(s) in the blood in the absence of the vaccine.

### C. Methods for Determining Immune Responses

Methods for determining immune responses are known in the art. In some embodiments, the immune response is measured by detecting and/or quantifying the relative amount of an antibody, which specifically recognizes an antigen in the sera of a subject who has been treated by administering the non-replicating intact influenza viruses, relative to the amount of the antibody in an untreated control subject.

Techniques for the assaying antibodies and antibody filters in a sample are known in the art and include, for example, sandwich assays, ELISA and ELISpot. Polyclonal sera are relatively easily prepared by injection of a suitable laboratory animal with an effective amount of the immune effector, or antigenic part thereof, collecting serum from the animal and isolating specific sera by any of the known immuno-adsorbent techniques. Antibodies produced by this method are utilizable in virtually any type of immunoassay.

The use of monoclonal antibodies in an immunoassay is preferred because of the ability to produce them in large quantities and the homogeneity of the product. The preparation of hybridoma cell lines for monoclonal antibody production derived by fusing an immortal cell line and lymphocytes sensitized against the immunogenic preparation can be achieved by techniques which are well known to those who are skilled in the art. In other embodiments, ELISA assays may be used to determine the level of isotype specific antibodies using methods known in the art.

CTL assays can be used to determine the lytic activity of CTLs, measuring specific lysis of target cells expressing a certain antigen. Immune-assays may be used to measure the activation (e.g., degree of activation) of sample immune cells. Sample immune cells refer to immune cells contained in samples from any source, including from a human patient, human donor, animal, or tissue cultured cell line. The immune cell sample can be derived from peripheral blood, lymph nodes, bone marrow, thymus, any other tissue source including in situ or excised tumor, or from tissue or organ cultures. The sample may be fractionated or purified to generate or enrich a particular immune cell subset before analysis. The immune cells can be separated and isolated from their source by standard techniques.

Immune cells include both non-resting and resting cells, and cells of the immune system that may be assayed, including, but not limited to, B lymphocytes, T lymphocytes, natural killer (NK) cells, lymphokine-activated killer (LAK) cells, monocytes, macrophages, neutrophils, granulocytes, mast cells, platelets, Langerhans cells, stem cells, dendritic cells, and peripheral blood mononuclear cells.

Immune cell activity that may be measured include, but is not limited to (1) cell proliferation by measuring the cell or DNA replication; (2) enhanced cytokine production, including specific measurements for cytokines, such as γIFN, GM-CSF, or TNF-alpha, IFN-alpha, IL-6, IL-10, IL-12; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; (9) chemokine secretion such as IP-10; (10) expression of costimulatory molecules (e.g., CD80, CD 86) and maturation molecules (e.g., CD83), (11) upregulation of class II MHC expression; and (12) apoptosis, which refers to fragmentation of activated immune cells under certain circumstances, as an indication of abnormal activation.

Flow cytometry can also be used to measure proliferation by measuring DNA with light scatter, Coulter volume and fluorescence, all of which are techniques that are well known in the art.

Enhanced Cytokine Production Assay: A measure of immune cell stimulation is the ability of the cells to secrete cytokines, lymphokines, or other growth factors. Cytokine production, including specific measurements for cytokines, such as γIFN, GM-CSF, or TNF-alpha, may be made by radioimmunoassay (RIA), enzyme-linked immunoabsorbent assay (ELISA), bioassay, or measurement of messenger RNA levels. In general, with these immunoassays, a monoclonal antibody to the cytokine to be measured is used to specifically bind to and thus identify the cytokine. Immunoassays are well known in the art and can include both competitive assays and immunometric assays, such as forward sandwich immunoassays, reverse sandwich immunoassays and simultaneous immunoassays.

In each of the above assays, the sample-containing cytokine is incubated with the cytokine-specific monoclonal antibody under conditions and for a period of time sufficient to allow the cytokines to bind to the monoclonal antibodies. In general, it is desirable to provide incubation conditions sufficient to bind as much cytokine and antibody as possible, since this will maximize the signal. Of course, the specific concentrations of antibodies, the temperature and time of incubation, as well as other such assay conditions, can be varied, depending upon various factors including the concentration of cytokine in the sample, the nature of the sample, and the like. Those skilled in the art will be able to determine operative and optimal assay conditions for each determination by employing routine experimentation.

Immune cells express a variety of cell surface molecules which can be detected with either monoclonal antibodies or polyclonal antisera. Immune cells that have undergone differentiation or activation can also be enumerated by staining for the presence of characteristic cell surface proteins by direct immunofluorescence in fixed smears of cultured cells.

The above-described methods and other additional methods to determine an immune response are well known in the art.

D. Additional Agents to Administer with Replication-Deficient Influenza Viruses

In some forms, the method(s) for treating or preventing infections (or diseases described herein may be used in combination with one or more anti-bacterial agents, anti-viral agents, anti-fungal agents, or anti-protozoal agents.

### V. Kits

In some forms, the intact, replication-deficient influenza viruses are provided within a kit. An exemplary kit includes one or more containers filled with one or more of the following components: a live, modified, intact, replication-deficient influenza virus, and optionally including an additional active agent, such as an adjuvant, immune modulatory, or co-stimulatory molecule. The agents are typically either in dried form (e.g., lyophilized), as a salt, or in a solution, or optionally with a solution or gel to dissolve or admix the intact, replication-deficient influenza viruses. In some forms, the kits additionally contain a device for intradermal administration. Associated with such a kit can be instructions on how to use the kit and optionally a notice in the form prescribed by a governmental agency regulating the manufacture, use or sale of pharmaceuticals or biological products, which notice reflects approval by the agency of manufacture, use or sale for human administration.

The disclosed compositions and methods can be further understood through the following numbered paragraphs:

1. An intact, replication-deficient virus including an influenza virus genome, wherein the virus infects normal human cells,
wherein the influenza virus genome includes one or more mutations in one or more genes selected from the group including viral RNA polymerase PB1, viral RNA polymerase PB2, viral RNA polymerase PA, and nucleoprotein (NP), and
wherein the one or more mutations prevents or reduces replication of the virus in normal human cells by at least 90% as compared to the same virus in the absence of the mutation.

2. The replication-deficient virus of paragraph 1, wherein the influenza virus genome includes one or more mutations in the viral RNA polymerase PB2 gene that prevents or reduces replication of the virus in normal human cells.

3. The replication-deficient virus of paragraph 1 or 2, wherein the virus is 100% non-replicating in normal mammalian cells.

4. The replication-deficient virus of any one of paragraphs 1-3, wherein the influenza virus genome includes a viral RNA polymerase PB2 gene having the nucleic acid sequence of SEQ ID NO: 1, or a nucleic acid sequence having at least 75% identity to SEQ ID NO: 1.

5. The replication-deficient virus of any one of paragraphs 1-3, wherein the influenza virus genome includes a viral RNA polymerase PB2 gene having the nucleic acid sequence of SEQ ID NO:9, or a nucleic acid sequence having at least 75% identity to SEQ ID NO: 9.

6. The replication-deficient virus of any one of paragraphs 1-3, wherein the influenza virus genome includes a viral RNA polymerase PB2 gene having the nucleic acid sequence of SEQ ID NO: 10, or a nucleic acid sequence having at least 75% identity to SEQ ID NO: 10.

7. The replication-deficient virus of any one of paragraphs 1-3, wherein the influenza virus genome includes a viral RNA polymerase PB2 gene having the nucleic acid sequence of SEQ ID NO: 11, or a nucleic acid sequence having at least 75% identity to SEQ ID NO: 11.

8. The replication-deficient virus of any one of paragraphs 1-3, wherein the influenza virus genome includes a viral RNA polymerase PB2 gene having the nucleic acid sequence of SEQ ID NO: 12, or a nucleic acid sequence having at least 75% identity to SEQ ID NO: 12.

9. The replication-deficient virus of any one of paragraphs 1-3, wherein the influenza virus genome includes a viral RNA polymerase PB2 gene having the nucleic acid sequence of SEQ ID NO: 13, or a nucleic acid sequence having at least 75% identity to SEQ ID NO: 13.

10. The replication-deficient virus of any one of paragraphs 1-3, wherein the influenza virus genome includes a viral RNA polymerase PB2 gene having the nucleic acid sequence of SEQ ID NO:14, or a nucleic acid sequence having at least 75% identity to SEQ ID NO: 14.

11. The replication-deficient virus of any one of paragraphs 1-10, wherein the influenza virus genome includes a viral RNA polymerase PB2 gene having the nucleic acid sequence of SEQ ID NO: 15, or a nucleic acid sequence having at least 75% identity to SEQ ID NO: 15.

12. The replication-deficient virus of any one of paragraphs 1-11, wherein the influenza virus genome includes one or more mutations in the viral RNA polymerase PA gene that prevents or reduces replication of the virus in normal human cells.

13. The replication-deficient virus of any one of paragraphs 1-11, wherein the influenza virus genome includes a viral RNA polymerase PA gene having the nucleic acid sequence of SEQ ID NO: 16, or a nucleic acid sequence having at least 75% identity to SEQ ID NO: 16.

14. The replication-deficient virus of any one of paragraphs 1-11, wherein the influenza virus genome includes a viral RNA polymerase PA gene having the nucleic acid sequence of SEQ ID NO: 17, or a nucleic acid sequence having at least 75% identity to SEQ ID NO: 17.

15. The replication-deficient virus of any one of paragraphs 1-11, wherein the influenza virus genome includes a viral RNA polymerase PA gene having the nucleic acid sequence of SEQ ID NO: 18, or a nucleic acid sequence having at least 75% identity to SEQ ID NO: 18.

16. The replication-deficient virus of any one of paragraphs 1-11, wherein the influenza virus genome includes a viral RNA polymerase PA gene having the nucleic acid sequence of SEQ ID NO: 19, or a nucleic acid sequence having at least 75% identity to SEQ ID NO: 19.

17. The replication-deficient virus of any one of paragraphs 1-16, wherein the influenza virus genome includes one or more mutations in the viral RNA polymerase PB1 gene that prevents or reduces replication of the virus in normal human cells.

18. The replication-deficient virus of any one of paragraphs 1-17, wherein the influenza virus genome includes a viral RNA polymerase PB1 gene having the nucleic acid sequence of SEQ ID NO:20, or a nucleic acid sequence having at least 75% identity to SEQ ID NO: 20.

19. The replication-deficient virus of any one of paragraphs 1-17, wherein the influenza virus genome includes a viral RNA polymerase PB1 gene having the nucleic acid sequence of SEQ ID NO:21, or a nucleic acid sequence having at least 75% identity to SEQ ID NO: 21.

20. The replication-deficient virus of any one of paragraphs 1-17, wherein the influenza virus genome includes a viral RNA polymerase PB1 gene having the nucleic acid sequence of SEQ ID NO:22, or a nucleic acid sequence having at least 75% identity to SEQ ID NO: 22.

21. The replication-deficient virus of any one of paragraphs 1-17, wherein the influenza virus genome includes a viral RNA polymerase PB1 gene having the nucleic acid sequence of SEQ ID NO:23, or a nucleic acid sequence having at least 75% identity to SEQ ID NO: 23.

22. The replication-deficient virus of any one of paragraphs 1-17, wherein the influenza virus genome includes a viral RNA polymerase PB1 gene having the nucleic acid sequence of SEQ ID NO:23, or a nucleic acid sequence having at least 75% identity to SEQ ID NO: 23.

23. The replication-deficient virus of any one of paragraphs 1-17, wherein the influenza virus genome includes a viral RNA polymerase PB1 gene having the nucleic acid sequence of SEQ ID NO:24, or a nucleic acid sequence having at least 75% identity to SEQ ID NO: 24.

24. The replication-deficient virus of any one of paragraphs 1-17, wherein the influenza virus genome includes a viral RNA polymerase PB1 gene having the nucleic acid sequence of SEQ ID NO:25, or a nucleic acid sequence having at least 75% identity to SEQ ID NO: 25.

25. The replication-deficient virus of any one of paragraphs 1-17, wherein the influenza virus genome includes a viral RNA polymerase PB1 gene having the nucleic acid sequence of SEQ ID NO:26, or a nucleic acid sequence having at least 75% identity to SEQ ID NO: 26.

26. The replication-deficient virus of any one of paragraphs 1-17, wherein the influenza virus genome includes a viral RNA polymerase PB1 gene having the nucleic acid sequence of SEQ ID NO:27, or a nucleic acid sequence having at least 75% identity to SEQ ID NO: 27.

27. The replication-deficient virus of any one of paragraphs 1-26, wherein the influenza virus genome includes eight genomic segments,
wherein between one and seven of the genomic segments are derived from a first influenza virus, and
wherein between one and seven of the genomic segments are derived from a second influenza virus, and
wherein the one or more mutations that prevent or reduce viral replication are present in the genomic segments derived from the second virus.

28. The replication-deficient virus of paragraph 27, wherein the genome includes between five and seven genomic segments of the first influenza virus, and wherein the first virus is a replication-competent influenza A virus selected from the group including H1, H2, H3, H5, H6, H7, H9, and H10 subtypes.

29. The replication-deficient virus of paragraph 28, wherein the first virus is selected from the group including N1, N2, N6, N7, N8 and N9 subtypes.

30. The intact, non-replicating virus of any one of paragraphs 27 to 29, wherein the second virus is an influenza virus selected from the group including H1, H2, H3, H5, H6, H7, H9, and H10 subtypes.

31. The intact, non-replicating virus of paragraph 30, wherein the second virus is selected from the group including N1, N2, N6, N7, N8 and N9 subtypes.

32. An intact, replication-deficient virus including an influenza virus genome,
wherein the virus infects normal human cells,
wherein the influenza virus genome includes eight genomic segments,
wherein between one and seven of the genomic segments are derived from a first influenza virus,
wherein between one and seven of the genomic segments are derived from a second influenza virus,
wherein the influenza virus genome includes one segment including a viral RNA polymerase PB1 gene and one segment including a viral RNA polymerase PB2 gene,
wherein the influenza virus genome includes one or more mutations in the viral RNA polymerase PB1 gene and one or more mutations in the viral RNA polymerase PB2 gene,
wherein the one or more mutations prevent or reduce replication of the virus in normal human cells by at least 90% as compared to the same virus in the absence of the mutation.

33. The replication-deficient virus of paragraph 32, wherein the viral RNA polymerase PB1 gene has the nucleic acid sequence of SEQ ID NO:20, or a nucleic acid sequence having at least 75% identity to SEQ ID NO:20.

34. The replication-deficient virus of paragraph 32, wherein the viral RNA polymerase PB1 gene has the nucleic acid sequence of SEQ ID NO:21, or a nucleic acid sequence having at least 75% identity to SEQ ID NO:21.

35. The replication-deficient virus of paragraph 32, wherein the viral RNA polymerase PB1 gene has the nucleic acid sequence of SEQ ID NO:22, or a nucleic acid sequence having at least 75% identity to SEQ ID NO:22.

36. The replication-deficient virus of paragraph 32, wherein the viral RNA polymerase PB1 gene has the nucleic acid sequence of SEQ ID NO:23 or a nucleic acid sequence having at least 75% identity to SEQ ID NO:23.

37. The replication-deficient virus of paragraph 32, wherein the viral RNA polymerase PB1 gene has the nucleic acid sequence of SEQ ID NO:24, or a nucleic acid sequence having at least 75% identity to SEQ ID NO:24.

38. The replication-deficient virus of paragraph 32, wherein the viral RNA polymerase PB1 gene has the nucleic acid sequence of SEQ ID NO:25, or a nucleic acid sequence having at least 75% identity to SEQ ID NO:25.

39. The replication-deficient virus of paragraph 32, wherein the viral RNA polymerase PB1 gene has the nucleic acid sequence of SEQ ID NO:26, or a nucleic acid sequence having at least 75% identity to SEQ ID NO:26.

40. The replication-deficient virus of paragraph 32, wherein the viral RNA polymerase PB1 gene has the nucleic acid sequence of SEQ ID NO:27, or a nucleic acid sequence having at least 75% identity to SEQ ID NO:27.

41. The replication-deficient virus of any one of paragraphs 32-40, wherein the viral RNA polymerase PB2 gene has the nucleic acid sequence of SEQ ID NO:1, or a nucleic acid sequence having at least 75% identity to SEQ ID NO:1.

42. The replication-deficient virus of any one of paragraphs 32-40, wherein the viral RNA polymerase PB2 gene has the nucleic acid sequence of SEQ ID NO:9, or a nucleic acid sequence having at least 75% identity to SEQ ID NO:9.

43. The replication-deficient virus of any one of paragraphs 32-40, wherein the viral RNA polymerase PB2 gene has the nucleic acid sequence of SEQ ID NO:10, or a nucleic acid sequence having at least 75% identity to SEQ ID NO:10.

44. The replication-deficient virus of any one of paragraphs 32-40, wherein the viral RNA polymerase PB2 gene has the nucleic acid sequence of SEQ ID NO:11, or a nucleic acid sequence having at least 75% identity to SEQ ID NO:11.

45. The replication-deficient virus of any one of paragraphs 32-40, wherein the viral RNA polymerase PB2 gene has the nucleic acid sequence of SEQ ID NO:12, or a nucleic acid sequence having at least 75% identity to SEQ ID NO: 12.

46. The replication-deficient virus of any one of paragraphs 32-40, wherein the viral RNA polymerase PB2 gene has the nucleic acid sequence of SEQ ID NO: 13, or a nucleic acid sequence having at least 75% identity to SEQ ID NO:13.

47. The replication-deficient virus of any one of paragraphs 32-40, wherein the viral RNA polymerase PB2 gene has the nucleic acid sequence of SEQ ID NO:14, or a nucleic acid sequence having at least 75% identity to SEQ ID NO:14.

48. The replication-deficient virus of any one of paragraphs 32-40, wherein the viral RNA polymerase PB2 gene has the nucleic acid sequence of SEQ ID NO:15, or a nucleic acid sequence having at least 75% identity to SEQ ID NO:15.

49. The intact, non-replicating virus of any one of paragraphs 32-48, wherein the first influenza virus is a replication-competent influenza A virus selected from the group including H1, H2, H3, H5, H6, H7, H9, and H10 subtypes.

50. The intact, non-replicating virus of any one of paragraphs 32-49, wherein the first virus is selected from the group including N1, N2, N6, N7, N8 and N9 subtypes.

51. The intact, non-replicating virus of any one of paragraphs 32-50, wherein the second virus is an influenza virus selected from the group including H1, H2, H3, H5, H6, H7, H9, and H10 subtypes.

52. The intact, non-replicating virus of any one of paragraphs 32 to 51, wherein the second virus is selected from the group including N1, N2, N6, N7, N8 and N9 subtypes.

53. The intact, non-replicating virus of any one of paragraphs 32-52, wherein the influenza virus genome further includes one or more mutations in the one or more genes selected from viral RNA polymerase PA, and nucleoprotein (NP).

54. The replication-deficient virus of paragraph 53, wherein the influenza virus genome includes a viral RNA polymerase PA gene having the nucleic acid sequence of SEQ ID NO: 16, or a nucleic acid sequence having at least 75% identity to SEQ ID NO:16.

55. The replication-deficient virus of paragraph 53, wherein the influenza virus genome includes a viral RNA polymerase PA gene having the nucleic acid sequence of SEQ ID NO: 17, or a nucleic acid sequence having at least 75% identity to SEQ ID NO:17.

56. The replication-deficient virus of paragraph 53, wherein the influenza virus genome includes a viral RNA polymerase PA gene having the nucleic acid sequence of SEQ ID NO: 18, or a nucleic acid sequence having at least 75% identity to SEQ ID NO:18.

57. The replication-deficient virus of paragraph 53, wherein the influenza virus genome includes a viral RNA polymerase PA gene having the nucleic acid sequence of SEQ ID NO: 19, or a nucleic acid sequence having at least 75% identity to SEQ ID NO:19.

58. The intact, non-replicating virus of any one of paragraphs 32 to 57, wherein the genome includes between five and seven genomic segments of the first influenza virus.

59. The intact, non-replicating virus of any one of paragraphs 27-58, wherein the first and second viruses are of different subtypes.

60. The intact, non-replicating virus of any one of paragraphs 27-58, wherein the first and second viruses are different strains of the same subtype.

61. The replication-deficient virus of any one of paragraphs 32-60, wherein the virus is 100% non-replicating in normal mammalian cells.

62. The replication-deficient virus of any one of paragraphs 27-61, wherein the first influenza virus is selected from the group including H1N1, H3N1, H5N1, H7N9 and H2N2 subtypes.

63. The replication-deficient virus of any one of paragraphs 27-62, wherein the first or second influenza virus is A/WSN/1933 (H1N1).

64. The replication-deficient virus of any one of paragraphs 27-62, wherein the first or second influenza virus is A/PR8/34 (H1N1).

65. The replication-deficient virus of any one of paragraphs 27-62, wherein the first or second influenza virus is A/HK/415742/2009 (H1N1).

66. The replication-deficient virus of any one of paragraphs 27-62, wherein the first or second influenza virus is A/HK/4801/2014 (H3N2).

67. The replication-deficient virus of any one of paragraphs 27-66, wherein the genome includes between one and three genomic segments derived from the second influenza virus, and wherein the second virus is selected from the group including H5N1 and H7N9 subtypes.

68. The replication-deficient virus of any one of paragraphs 1-67, wherein the genome includes a mutated PB1, PB2 and/or PA gene derived from an H7N9 virus.

69. The replication-deficient virus of any one of paragraphs 1-68, wherein the virus includes one or more exogenous genes derived from a defective-interfering (DI) particle.

70. An intact, non-replicating virus including an influenza virus genome,
wherein the virus infects normal human cells,
wherein the influenza virus genome comprises
   (i) genomic segments 4-8 of the A/WSN/1933 (H1N1) genome having a nucleic acid sequence of SEQ ID NOS: 4-8; and
   (ii) genomic segment 1 including a mutated PB2 gene having a nucleic acid sequence of any one of SEQ ID NOs: 1, or 9-15, or a nucleic acid sequence having at least 75% identity to any one of SEQ ID NO: 1, or 9-15; and
   (iii) genomic segments 2 and 3 including the PB1 and PA genes of an H7N9 virus having a nucleic acid sequence of SEQ ID NOS: 2-3; and wherein the virus is completely non-replicating in normal human cells.

71. An intact, non-replicating virus including an influenza virus genome,
wherein the virus infects normal human cells,
wherein the influenza virus genome comprises
   (i) genomic segments 4 and 6 of the A/HK/4801/2014 (H3N2) genome; and
   (ii) genomic segment 1 including a mutated PB2 gene having a nucleic acid sequence of SEQ ID NO:11, 13 or 15, or a nucleic acid sequence having at least 75% identity to SEQ ID NO 11, 13 or 15, and
   (iii) genomic segments 2 and 3 including the PB1 and PA genes of an H7N9 virus having a nucleic acid sequence of SEQ ID NOS: 2-3; and
wherein the virus is completely non-replicating in normal human cells.

72. The intact, non-replicating virus of paragraph 71, including genomic segments 7 and 8 from A/PR8/34 (H1N1).

73. An intact, non-replicating virus including an influenza virus genome,
wherein the virus infects normal human cells,
wherein the influenza virus genome comprises
   (i) genomic segments 4 and 6-8 of the A/HK/415742/2009 (H1N1) genome; and
   (ii) genomic segment 1 including a mutated PB2 gene having a nucleic acid sequence of SEQ ID NO:13, or a nucleic acid sequence having at least 75% identity to SEQ ID NO:13, and
   (iii) genomic segments 2 and 3 including the PB1 and PA genes of an H7N9 virus having a nucleic acid sequence of SEQ ID NOS: 2-3; and
wherein the virus is completely non-replicating in normal human cells.

74. An intact, non-replicating virus including an influenza virus genome,
wherein the virus infects normal human cells,
wherein the influenza virus genome comprises
   (i) genomic segments 4 and 6-8 of the A/HK/415742/2009 (H1N1) genome; and
   (ii) genomic segment 2 including a mutated PB1 gene having a nucleic acid sequence of SEQ ID NO:26 or 27, or a nucleic acid sequence having at least 75% identity to SEQ ID NO:26 or 27, and
   (iii) genomic segments 1 and 3 including the PB2 and PA genes of an H7N9 virus; and
wherein the virus is completely non-replicating in normal human cells.

75. An intact, non-replicating virus including an influenza virus genome,
wherein the virus infects normal human cells,
wherein the influenza virus genome comprises
   (i) genomic segments 4 and 6-8 of the A/PR8/34 (H1N1) genome; and
   (ii) genomic segment 2 including a mutated PB1 gene having a nucleic acid sequence of SEQ ID NO:22, or a nucleic acid sequence having at least 75% identity to SEQ ID NO:22,
   (iii) genomic segment 1 including a mutated PB2 gene having a nucleic acid sequence of SEQ ID NO:14, or a nucleic acid sequence having at least 75% identity to SEQ ID NO:14, and
   (iv) genomic segment 3 including the PA gene of an H7N9 virus having a nucleic acid sequence of SEQ ID NO:3; and
wherein the virus is completely non-replicating in normal human cells.

76. An intact, non-replicating virus including an influenza virus genome,
wherein the virus infects normal human cells,
wherein the influenza virus genome comprises
   (i) genomic segments 4 and 6-8 of the A/PR8/34 (H1N1) genome; and
   (ii) genomic segment 1 including a mutated PB1 gene having a nucleic acid sequence of SEQ ID NO:1, or a nucleic acid sequence having at least 75% identity to SEQ ID NO:1, and
   (iii) genomic segments 2 and 3 including a PB2 and PA genes of an H7N9 virus having a nucleic acid sequence of SEQ ID NO:2 and 3, respectively; and
wherein the virus is completely non-replicating in normal human cells.

77. An intact, non-replicating virus including an influenza virus genome,
wherein the virus infects normal human cells,
wherein the influenza virus genome comprises
   (i) genomic segments 4 and 6-8 of the A/PR8/34 (H1N1) genome; and
   (ii) genomic segment 2 including a mutated PB1 gene having a nucleic acid sequence of SEQ ID NO:22, or a nucleic acid sequence having at least 75% identity to SEQ ID NO:22,
   (iii) genomic segments 1 and 3 including the PA gene of an H7N9 virus; and
wherein the virus is completely non-replicating in normal human cells.

78. An intact, non-replicating virus including an influenza virus genome,
wherein the virus infects normal human cells,
wherein the influenza virus genome comprises
   (i) genomic segments 4-8 of the A/WSN/1933 (H1N1) genome having a nucleic acid sequence of SEQ ID NOS: 4-8; and
   (ii) genomic segment 1 including a mutated PB2 gene having a nucleic acid sequence of SEQ ID NO:13, or a nucleic acid sequence having at least 75% identity to any one of SEQ ID NO: 13;
   (iii) genomic segment 2 including a mutated PB1 gene having a nucleic acid sequence of SEQ ID NO:22, or a nucleic acid sequence having at least 75% identity to any one of SEQ ID NO:22; and
   (iv) genomic segment 3 including the PA gene of an H7N9 virus having a nucleic acid sequence of SEQ ID NO:3; and
wherein the virus is completely non-replicating in normal human cells.

79. An intact, non-replicating virus including an influenza virus genome,
wherein the virus infects normal human cells,
wherein the influenza virus genome comprises
   (i) genomic segments 4-8 of the A/WSN/1933 (H1N1) genome having a nucleic acid sequence of SEQ ID NOS: 4-8; and
   (ii) genomic segment 2 including a mutated PB1 gene having a nucleic acid sequence of SEQ ID NO:22, or a nucleic acid sequence having at least 75% identity to any one of SEQ ID NO:22; and
   (iii) genomic segments 1 and 3 including the PA gene of an H7N9 virus; and
wherein the virus is completely non-replicating in normal human cells.

80. The intact, non-replicating virus of any one of paragraphs 1-79, wherein the virus morphology includes a spherical or elliptical virion having a diameter of between about 80 nm and about 120 nm, inclusive.

81. A vaccine composition for providing immunity to influenza viruses in a subject, including
(i) the intact, replication-deficient virus of any one of paragraphs 1-80, and
(ii) a pharmaceutically acceptable excipient suitable for intradermal administration,
wherein the composition is in an amount effective to induce a protective immune response to one or more influenza viruses in the subject following intradermal administration to the subject.

82. The vaccine composition of paragraph 81, wherein the composition is in an amount effective to induce a protective immune response to one or more of an H1/N1, H3/N2 or H5/N1 influenza virus.

83. The vaccine composition of paragraph 81 or 82, further including one or more additional agents selected from group including co-stimulatory molecules, growth factors, adjuvants, and cytokines.

84. The vaccine composition of paragraph 83, wherein the additional agent is selected from the group including IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Flt3-ligand, and CCR4.

85. A method for inducing or stimulating a protective immune response to an influenza virus in a subject, including administering to epidermal tissues of the subject the vaccine composition of any one of paragraphs 81-84, in an amount effective to induce or stimulate the immune response in the subject.

86. The method of paragraph 85, wherein the vaccine composition is administered to the subject by intradermal injection.

87. The method of paragraph 85 or 86, further including administering to the subject one or more additional agents selected from the group including an anti-infective agent, a co-stimulatory molecule, a growth factor, an adjuvant and/or cytokine,
wherein the one or more additional agents are administered before, at the same time, or after administering the vaccine composition.

88. The method of paragraph 87, wherein the additional agent is selected from the group including IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Flt3-ligand, and CCR4.

89. The method of any one of paragraphs 85-88, including repeating the step of administering the vaccine composition to the subject.

90. The method of paragraph 89, wherein the repeated administration is carried out at a time of one, two, three, four, five, six, seven, eight, nine, or ten days or weeks after the first administration.

91. The method of any one of paragraphs 85-90, wherein the method provides protective immunity to two or more different strains of influenza viruses.

92. The method of any one of paragraphs 85-91, wherein the method provides protective immunity to one or more H1N1 influenza viruses and one or more H3N2 influenza viruses.

93. The method of any one of paragraphs 85-92, wherein the method provides protective immunity to one or more H5N1 influenza viruses and/or one or more H7N9 influenza viruses.

94. A kit including the vaccine composition of any one of paragraphs 81-84 and optionally one or more devices for intradermal administration of the composition to a subject.

95. A dosage unit for immunization by intradermal administration including an effective amount of the vaccine composition of any one of paragraphs 81-84 for inducing or stimulating a protective immune response to an influenza virus in a subject.

96. A method of making an intact, replication-deficient virus including an influenza virus genome,
wherein the influenza virus genome includes one or more mutations in one or more genes selected from the group including viral RNA polymerase PB1, viral RNA polymerase PB2, viral RNA polymerase PA, and nucleoprotein (NP), including
   (a) introducing into a first cell genes encoding 4-7 segments of a wild-type virus; and
   (b) introducing into a second cell gene(s) encoding a mutant PB1, PB2, PA, and/or NP, and
   (c) co-culturing of the first and second cells; and
   (d) isolating the intact, replication-deficient virus.

97. The method of paragraph 96, wherein the first and/or second cell is selected from the group including a MDCK cell and a 293FT cell.

98. The method of paragraphs 96 or 97, wherein the mutant PB1, PB2, PA, and/or NP gene(s) is introduced into the cell by lentivirus transduction.

99. The method of any one of paragraphs 96 to 98, wherein the mutant PB1, PB2, PA, and/or NP gene(s) is within an expression vector which is driven by an RNA polymerase I promoter

100. The method of any one of paragraphs 96-99, wherein isolating in step (d) includes purification by filtration.

101. The method of paragraph 100, wherein the filtration includes passing through one or more 0.45 μm filter.

102. The method of any one of paragraphs 96-99, wherein isolating in step (d) includes purification by ultracentrifugation.

103. The method of paragraph 102, wherein the ultracentrifugation is sucrose-cushioned ultracentrifugation.

104. The method of paragraph 102 or 103, wherein the ultracentrifugation is at 28,000 rpm for 4 hours.

105. A cell expressing an intact, replication-deficient virus produced according to the method of any one of paragraphs 96-104.

106. A an intact, replication-deficient virus produced according to the method of any one of paragraphs 96-104.

The present invention is further illustrated by the following non-limiting examples. The entire contents of all of the references (including literature references, issued patents, published patent applications, and co-pending patent applications) cited throughout this application are hereby expressly incorporated by reference.

### EXAMPLES

### Example 1: Rational Design and Productive Synthesis of IDIV

The development and characterization of a novel live attenuated IntraDermal Influenza Vaccine, IDIV, a regimen combining the use of a rationally designed single-round infectious virus and intradermal route of administration are described.

### Methods

### Cell-Lines

293FT and MDCK cell-lines were obtained from Thermo Fisher Scientific and American Type Culture Collection respectively. 293FT cells were cultured in Dulbecco's Modified Eagle's Medium (DMEM) supplemented with 10% Fetal bovine serum (FBS) (Thermo Fisher Scientific), while MDCK cells were cultured in Minimum Essential Medium (MEM) (Thermo Fisher Scientific) supplemented with 10% FBS in 37° C. humidified incubator with 5% CO₂. To establish 293-PB2 and MDCK-PB2 cell-lines stably expressing PB2 protein of H7N9 (A/Zhejiang/01/2013), 293FT and MDCK cells were transduced with lentivirus encoding H7N9 PB2 transgene. To minimize the chance of recombinant between PB2 transgene and PB2 viral genome, 12 synonymous substitutions were introduced to both 5′ and 3′ ends of the coding sequence of PB2 transgene. Transduced cells were further selected for three days in complete medium supplemented with 2 μg/ml of puromycin (Thermo Fisher Scientific). Protein expression of PB2 in stable cell-lines was validated by Western blotting.

### Preparation of Human Blood-Derived Macrophages and Monocyte-Derived Dendritic Cells

Human peripheral blood of four donors were obtained from Blood Transfusion Service, Hong Kong Red Cross. Protocol was approved by the Institutional Review Board of the University of Hong Kong. Briefly, peripheral blood mononuclear cells (PBMCs) were isolated from buffy coat using Ficoll-Plaque reagent (GE Healthcare). Monocytes were further enriched using CD14 magnetic beads (Miltenyi Biotec). To generate peripheral blood-derived macrophages (PBDMs) or monocyte-derived Dendritic cells (MoDC), the CD14+ monocytes isolated were cultured for 6 days in RPMI with 10% FBS, supplemented with recombinant GM-CSF protein (Miltenyi Biotec) alone or recombinant GM-CSF plus recombinant IL-4 proteins (Miltenyi Biotec) respectively.

### Animals

Groups of six to eight-week-old female BALB/c mice were provided by Laboratory Animal Unit of The University of Hong Kong. Mice were housed in specific pathogen-free animal facility with 12 hr light-dark cycle and had free access to food and water. All mouse experiments strictly followed the procedures approved by Committee on the Use of Live Animals in Teaching and Research (CULATR), The University of Hong Kong.

### Generation of Wild-Type and Chimeric Viruses

Recombinant influenza H1N1 and its chimeric viruses harboring reassorted segments from WSN (A/WSN/1933), H7N9 (A/Zhejiang/01/2013) or H5N1 (A/VietNam/1203/2004) were generated by reverse genetics as previously described (Song, et al., Nat Commun 5, 5509 (2014); K. P. Mok, et al., J Infect Dis 200, 1104-1112 (2009)). Briefly, reverse genetics plasmids of indicated viral segments were co-transfected into 293 FT/MDCK co-culture. The co-cultured cells were then incubated for 2 to 3 days in plain MEM supplemented with TPCK-treated trypsin. All the wild-type and chimeric viruses were quantitated by both plaque assay and hemagglutination assay. Mouse-adapted pdm09 H1N1 (A/415742Md/Hong Kong/2009), which was used for microneutralization assay, was previously described (Li, et al., Front Immunol 9, 2370 (2018)).

### Production of IDIV

By applying reverse genetics, high-titer homogenous IDIV was successfully produced in 293-PB2 and MDCK-PB2 cells stably expressing PB2 protein. First, PB2-DI was inserted into an expression plasmid driven by RNA polymerase I promotor (pPolI-PB2-DI). Co-culture of 293-PB2 and MDCK-PB2 stable cells were co-transfected with pPoll-PB2-DI, pPoll plasmids for WSN segments (HA, NP, NA, M and NS) and bi-directional plasmids for H7N9 segments (PB1 and PA) using Lipofectamine 2000 (Thermo Fisher Scientific). Transfected cells were then maintained in plain MEM supplemented with TPCK-treated trypsin. Supernatant was collected at day 3 post-transfection. IDIV was then plaque-purified and propagated in MDCK-PB2 stable cells. Absence of replicative virus was confirmed by plaque assay using parental MDCK cells and reverse transcription-polymerase chain reaction. To further purify the IDIV, 0.45 μm-filtered DI virus was pelleted by ultra-centrifugation at 28,000 rpm for 4 hours at 4° C. against 25% sucrose bed using Optima XPN-100 ultracentrifuge (Beckman Coulter) and resuspended in sterile PBS. Aliquots of IDIV were stored at −80° C. freezer and quantitated by plaque assay using MDCK-PB2 cells.

### Serial Passaging of IDIV and RT-PCR

Purified IDIV was serially passaged five times. MDCK-PB2 cell-line was initially inoculated at MOI=0.001. Supernatant was collected when approximately 70% CPE is observed. The virus was further passaged for 4 times at 1:1000 dilution. Viral RNA was extracted from supernatant of each passage using QIAamp Viral RNA Mini Kit (Qiagen). Extracted vRNA was reverse transcribed using PrimeScript RT reagent kit with gDNA Eraser (Takara) with uni-12 primer 5′-AGCAAAAGCAGG-3′ (SEQ ID NO:32). PCR was performed with DreamTaq Green DNA Polymerase (Thermo Fisher Scientific) or PrimeSTAR GXL DNA Polymerase (Takara).

### Electron Microscopy

Recombinant IDIV was paraformaldehyde-fixed and concentrated by ultracentrifugation as described above. Absence of viable virions was confirmed by plaque assay on MDCK-PB2 cells. The downstream negative staining was serviced by Electron Microscopy Unit, The University of Hong Kong. Briefly, equal volume of 3% aqueous phosphotungstic acid was added to the sample. The mixture was then loaded onto 400 mesh carbon-formvar coated copper grids, UV-irradiated and air-dried. The morphology of virions was examination using Philips CM100 Transmission Electron Microscope.

### Mouse Immunization, Blood Sample Collection and Viral Infection

Mice were shaved at lower back one day prior to vaccination. On the day of vaccination, mouse anesthetized with ketamine (100 mg/kg of mouse body weight) and xylazine (10 mg/kg) was intradermally injected with a total of 100 μl of IDIV, 100 μl of PBS, or 100 μl of commercial quadrivalent vaccine Fluarix Tetra (GSK, UK) at three independent sites. At different time-point post-vaccination, blood was collected by facial vein puncture. Serum samples were aliquoted and stored at −80° C. freezer. At the indicated time post-immunization, mice were intranasally inoculated with 20 μl of diluted virus under anesthesia. Body weight and disease signs of infected mice were monitored daily for 14 days.

### Hemagglutination Microneutralization and Fluorescent Focus Microneutralization Assays

Mouse sera were treated with 3-time volume of receptor-destroying enzyme (RDE) II (Denka Seiken) at 37° C. for 20 hours, followed by inactivation at 56° C. for 30 min. The RDE-treated sera were then diluted with 6-time volume of PBS to attain 1:10 dilution, and further 2-fold serially diluted for 8 dilutions. For hemagglutination inhibition (HAI) assay, 25 μl of diluted RDE-treated sera were mixed with 25 μl of 4-HA-unit virus for 1 hour incubation at room temperature. WSN and pdm09 virus were used for IDIV and commercial quadrivalent vaccine respectively. After incubation, 50 μl of 0.5% Turkey red blood cells (Lampire Biological Laboratories) was added. Hemagglutination was recorded 45 min after incubation at room temperature. For microneutralization (MN) assay, 30 μl of serially diluted RDE-treated sera were mixed with 100 TCID50 of either WSN or pdm09 virus in 30 μl and incubated at room temperature for 1 hour. WSN and pdm09 virus were used for IDIV and commercial quadrivalent vaccine respectively. The virus-serum mix was added to PBS-washed MDCK cells in 96-well plates, followed by 1 hour incubation in 37° C. humidified incubator with 5% CO₂ to allow viral adsorption. The inoculum was then aspirated and replaced with MEM containing 1 μg/mL TPCK-treated trypsin and 100 U/ml of Penicillin-Streptomycin. The plates were incubated in 37° C. humidified incubator with 5% CO₂ for 3 days, followed by fixation by 4% paraformaldehyde. Adherent cells were visualized by 0.5% (w/v) Crystal Violet staining. For fluorescent focus microneutralization (FFMN) assay, 30 μL of RDE-treated sera were incubated with 30 μL of WSN or pdm09 virus at room temperature for 1 hour. MDCK cells seeded on chamber-slides were then infected with the serum-treated virus at MOI=1. After 5 hours, cells were fixed with 4% paraformaldehyde and permeabilized by NP-40. Cells were then stained with mouse anti-NP antibody (in-house) and Alexa Fluor® 488-conjungated donkey anti-mouse IgG antibody (Abcam) with DAPI counterstain. Percentage of green fluorescence-positive cells were counted under fluorescent microscope.

### Mouse Immunoglobulin Isotyping

Five classes of immunoglobulin in sera obtained from immunized mice were quantitated using multiplex bead-based immunoassay (LEGENDplex, BioLegend) according to the manufacturer's instruction. In brief, sera of immunized mice were incubated with a mix of capture beads for six immunoglobulins including IgG1, IgG2a, IgG2b, IgG3, IgA, and IgM. After washing, the samples were stained with biotinylated detection antibody cocktail followed by streptavidin-phycoerythrin (SA-PE). The intensity of PE was detected by fluorescence-activated cell sorting (FACS) (BD LSRFortessa) and the amount of each isotype antibody in the serum was determined according to the individual standard curves using LEGENDplex Data Analysis Software (BioLegend).

### Cytokine Profiling Assays

The cytokine profile in cell supernatant or mouse sera was determined by multiplex bead-based immunoassay (LEGENDplex, BioLegend) according to the manufacturer's instruction. In brief, cell supernatant or mouse sera was incubated with a mix of capture beads for various cytokines. After washing, the samples were stained with biotinylated detection antibody cocktail followed by streptavidin-phycoerythrin (SA-PE). The intensity of PE was detected by FACS (BD LSRFortessa) and the amount of each cytokine in the cell supernatant or sera was determined according to the individual standard curves using LEGENDplex Data Analysis Software (BioLegend).

### SMRT Sequencing

The library preparation of SMRT sequencing was performed as previously described (Lui, et al., Emerg Microbes Infect 8, 662-674 (2019)). Briefly, the viral RNA was extracted using QIAamp Viral RNA Mini Kit (Qiagen) and reverse transcribed. The viral genome was PCR-amplified, purified, and barcoded in the second round DNA amplification, followed by generation of circular single stranded DNA. One sequel SMRT cell was used to sequence 2 amplicons. The SMRTbell adapter sequences were removed, and CCS reads within 150-2400 bp were selected for further analysis. Sequences were aligned against the reference genome of H7N9 A/Anhui/1/2013 strain (EPI439503-5; EPI439508). The histograms of numbers of counts and length of read length were generated using R.

### Results

Influenza viruses were reported to produce defective-interfering (DI) particles, which are defective viruses that contain internal truncations in viral genome (Fazekas De St. Groth, and Graham, Nature 173, 637-638 (1954)). These viruses, despite being non-replicative due to the loss of essential viral gene expression, are able to enter cells in the same way as a standard virus does (Huang and Baltimore, Nature 226, 325-327 (1970)). Defective particle is therefore an ideal candidate of the IDIV, which has to be infectious but completely non-replicative. The obstacle for efficient IDIV production is that the presence of DI particles was only sporadically reported on certain laboratory strain viruses, and the amount of DI particles was often little when compared with the prototypic virus. However, in a previous study, abundant presence of defective interfering genomes was observed in influenza A/H7N9 virus-infected patient samples (Lui, et al., Emerg Microbes Infect 8, 662-674 (2019)).

To design a vaccine prototype that can be efficiently produced in vitro, the abundance of defective genomes was examined in a panel of chimeric A/H1N1 viruses. Each chimeric virus was constructed in A/H1N1/WSN backbone (a laboratory strain), but with the three polymerase subunits or nucleoprotein from either A/H1N1 (WSN), A/H7N9 or A/H5N1 (as illustrated in FIG. 1). Viral RNAs were extracted from viral supernatant and defective interfering genomes were visualized by RT-PCR and gel electrophoresis. As expected, large quantity of defective genome was detected in the viral stock of chimeric H1N1 virus possessing either H5N1 or H7N9 polymerases (H5-3P and H7-3P). Next the defective viral genome was analyzed using a recently established DI analysis platform that utilizes third-generation Single Molecule Real-Time (SMRT) long-read sequencing technology (Lui, et al., Emerg Microbes Infect 8, 662-674 (2019)). Using this platform, the sequence of the most abundant defective genome was precisely identified, which was presumably a stable species that can be efficiently replicated and packaged into virions. SMRT sequencing of viral defective genomes provided histograms depicting the size distribution of defective H7-3P genomes and H5-3P genome. Each peak in the histograms represented a unique defective genome sequence, while the height of the peak indicated its abundancy. The most abundantly expressed was H7-3P PB2 defective interfering genome with the size of 322 nucleotides long. The most abundantly expressed DI genome, which was derived from the A/H7N9 PB2 segment (named as PB2-DI), was chosen as the blueprint of the novel IDIV (FIG. 2A).

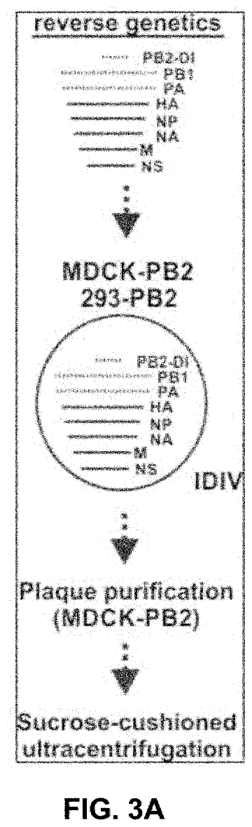

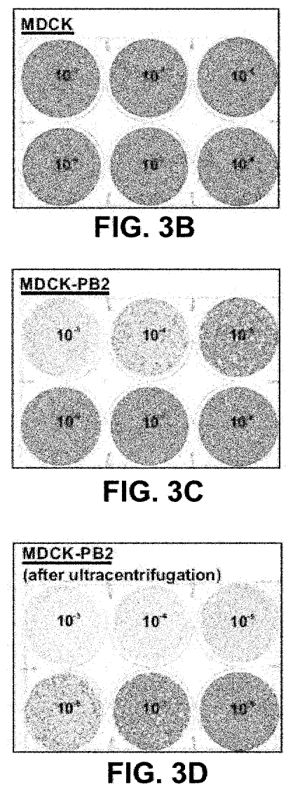

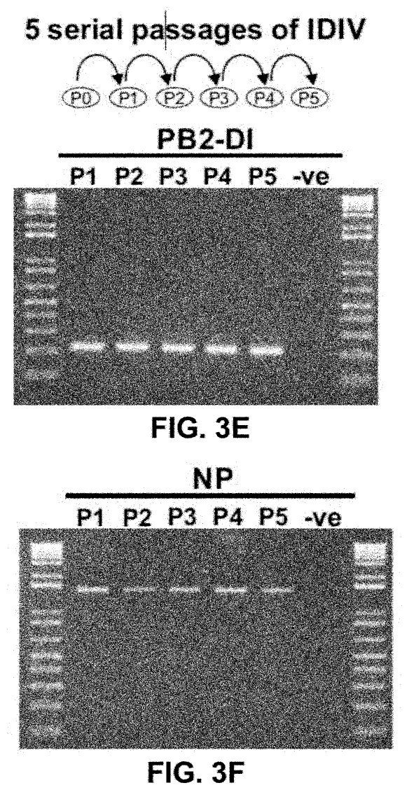

To produce high-titer non-replicating IDIV without contamination with standard virus, a modified reverse genetics system was used. Stable MDCK and 293 cells trans-complemented with H7N9 PB2 transgene were first established. 12 silent mutations next to the start and stop codons were introduced into the H7N9 PB2 transgene to minimize the chance of its recombination with the viral genome during IDIV production (FIGS. 2A and 2B). IDIV was then produced by transfecting the reverse genetic plasmids into MDCK-PB2/293-PB2 co-cultured cells, but with the pPolI-PB2 plasmid replaced with pPolI-PB2-DI. The DI virus produced was then plaque-purified and further propagated in MDCK-PB2 cells (FIGS. 3A-3F). The recombinant IDIV was completely replication-defective as evidenced by the absence of plaques in parental MDCK cells but present in MDCK-PB2 cells (FIGS. 3B-3D). After purification by sucrose-cushioned ultracentrifugation, titer of IDIV reached 2.5×10⁹ PFU/ml. Most importantly, purified IDIV remained infectious and stable over serial passaging. Plaque-purified IDIV was propagated for 5 serial passages (P1 to P5) in MDCK-PB2 cells. Stability of PB2-DI viral genome was evaluated by RT-PCR and gel electrophoresis (FIGS. 3E-3F). Neither revertant to full-length PB2 nor further truncation of PB2-DI could be observed. Negative-staining electron microscopy also showed that the virions of IDIV displayed morphology and size similar to wildtype virions, which were reported to be in spherical or elliptical shapes with 80-120 nm diameter (Noda, Front Microbiol 2, 269 (2011)).

### Potent Cytokine Induction by Single-Round IDIV Infection in Human Immune Cells

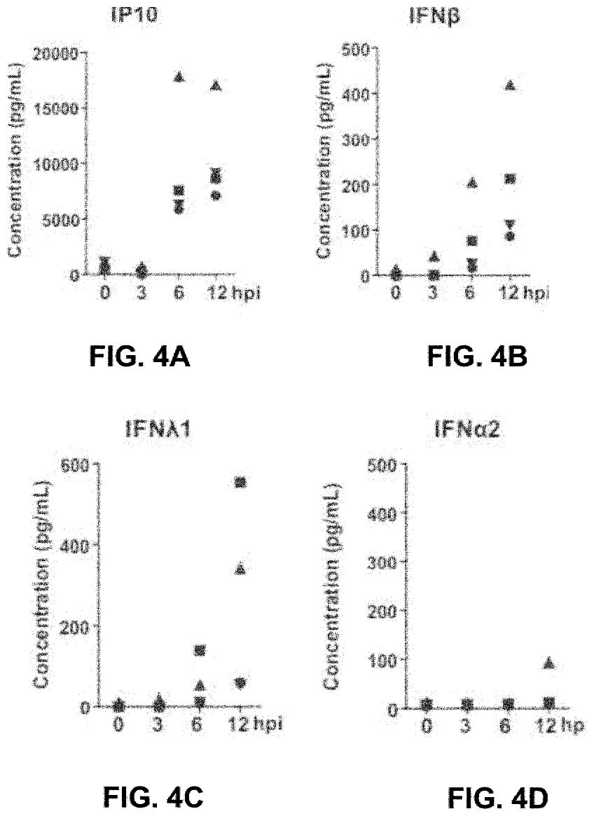

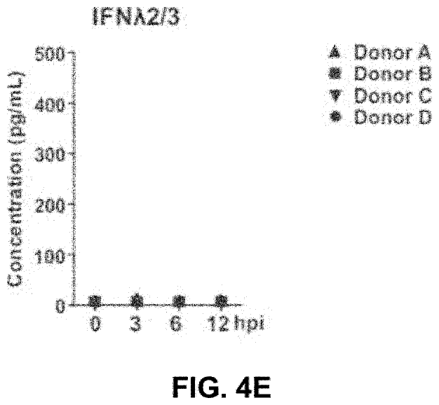

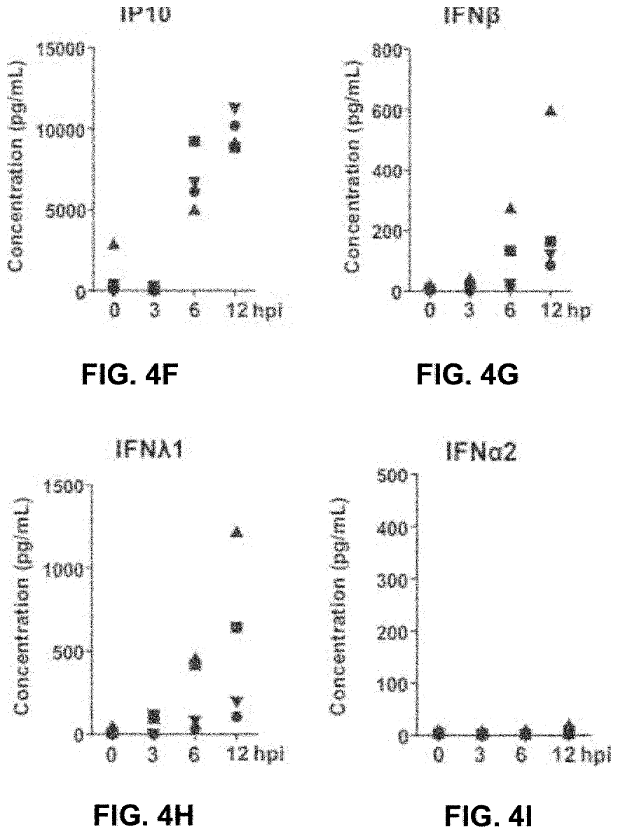

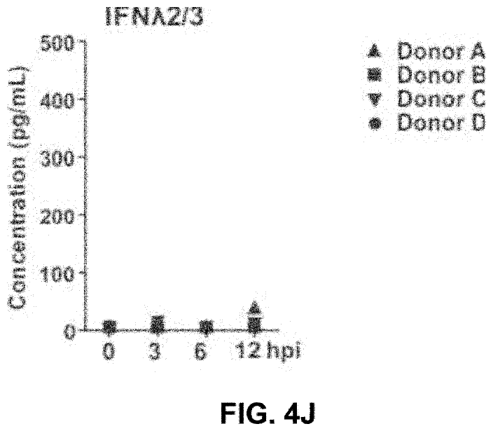

Innate immune response can be initiated by the recognition of influenza viral replicating genomes or small viral RNAs (Rehwinkel, et al., Cell 140, 397-408 (2010); Baum, et al., Proc Natl Acad Sci USA 107, 16303-16308 (2010)). It is unclear whether a single-round infectious IDIV is able to elicit innate immune response, which is important for successful immunization. To examine the immunostimulatory potential of IDIV in vitro, human peripheral blood-derived macrophages (PBDM) and monocyte-derived dendritic cells (MoDC) from four healthy donors were infected with IDIV. Activation of innate immune response in these antigen-presenting cells (APCs) was reflected by induction of cytokine secretion. Surprisingly, despite the complete abortion of viral shedding due to the lack of an intact PB2 viral genome and so PB2 protein expression, infection of IDIV was able to robustly induce the secretion of chemokine (IP10), type-I and type-III interferons (IFNB and IFNL1 respectively) in both PBDM (FIGS. 4A-4E) and MoDC (FIGS. 4F-4J). The single-rounded infection of IDIV, plus its robust activation of APCs hinted the worthiness of evaluating the potential use of IDIV as a novel vaccine.

### IDIV Elicits Expedited Seroconversion and Induces High-Titer Neutralizing Antibody Production in Mice

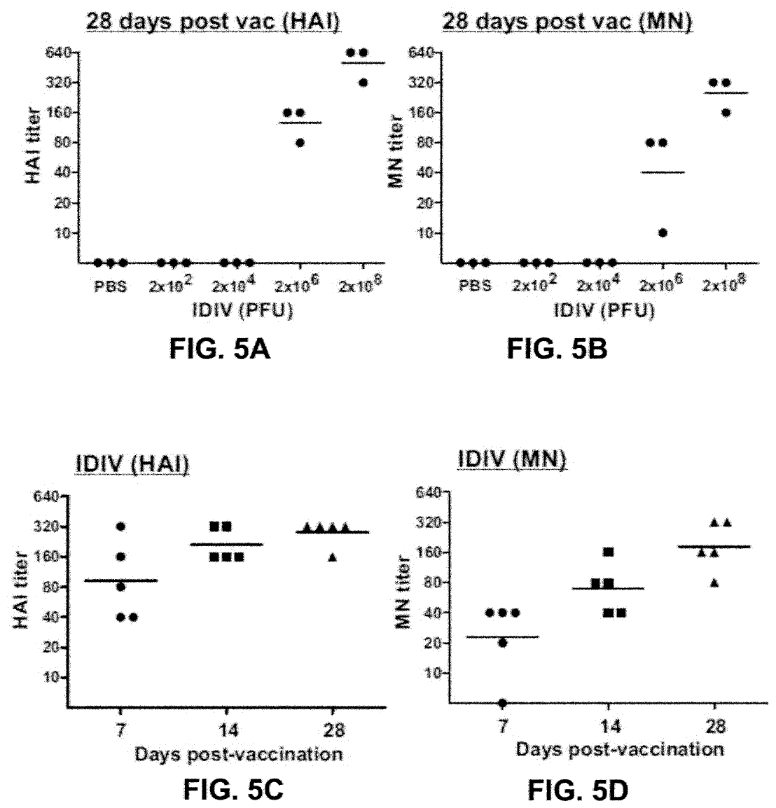

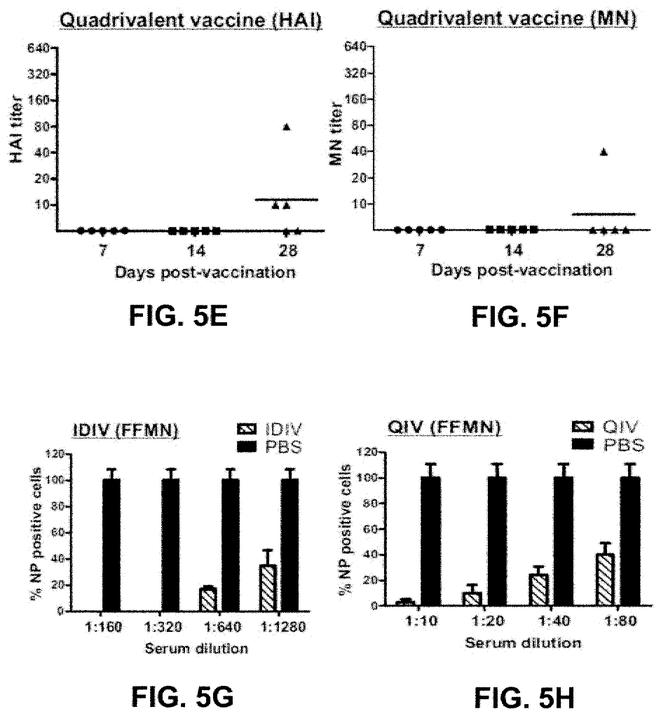

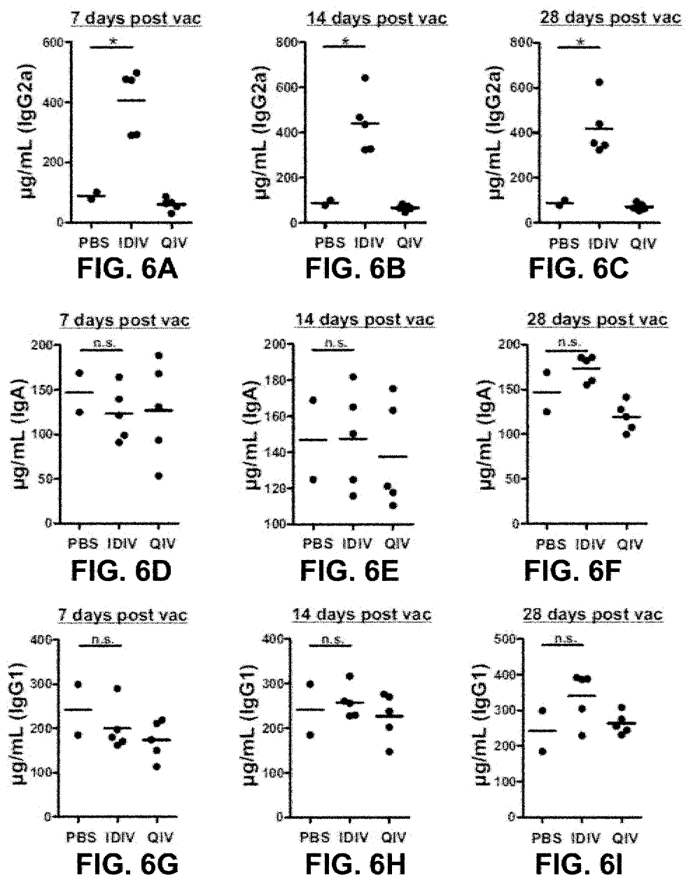

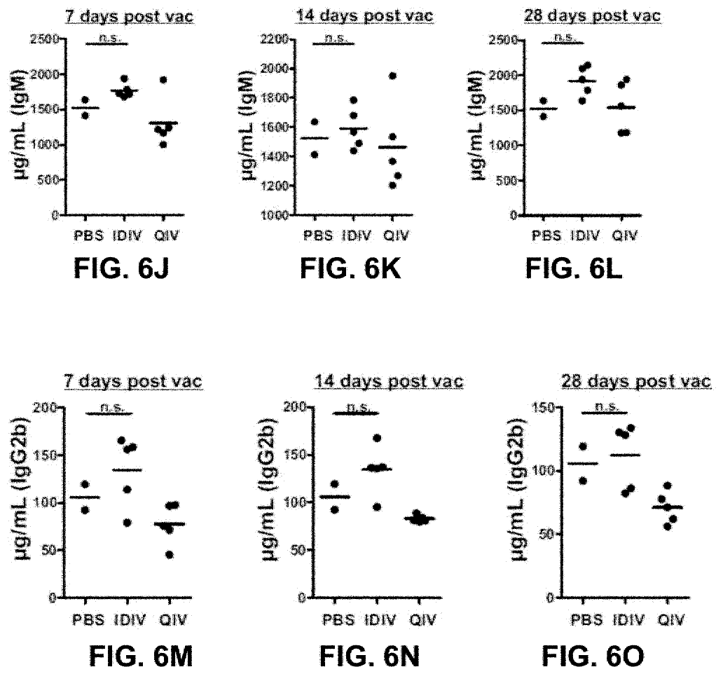

Dosing is one of the key determinants to vaccine efficacy. Since this is the first study applying a live but replication-defective influenza virus as intradermal vaccine, the serum neutralizing antibody production induced by various doses of IDIV in 6- to 8-week-old BALB/c mice was examined by hemagglutination inhibition (HAI) assay and microneutralization (MN) assay (FIGS. 5A and 5B). It was found that intradermal injection of 2×10⁶ or 2×10⁸ PFU (Plaque Forming Unit) of IDIV, respectively, triggered high-level (80-160 HAI) and extraordinarily high-level (320-640 HAI), respectively, neutralizing antibody production at 28 days post-vaccination. This remarkably strong seroconversion prompted further determination of the kinetics of neutralizing antibody production. Intradermal administration of 2×10⁸ PFU IDIV induced high-level of neutralizing antibody as early as 7 days post-vaccination (FIGS. 5C and 5D). Interestingly, when the induced serum antibody classes were profiled, IDIV was found to have specifically augmented production of IgG2a antibodies, (FIGS. 6A-6O) which were previously reported to be associated with improved protection against influenza infection (Huber, et al., Clin Vaccine Immunol 13, 981-990 (2006)). On the contrary, although immunization with commercial quadrivalent vaccine can protect mice against infection, neutralizing antibody production could only be moderately induced, indicating a sub-optimal immunization. After 28-day immunization, serum neutralizing antibodies in quadrivalent vaccine-immunized mice could only be detected by the more sensitive fluorescent focus microneutralization (FFMN) assay, but marginally by HAI and MN assays (FIGS. 5E-5H).

IDIV Protects Mice from Lethal H1N1 Infection as Early as 7-Day Post-Immunization

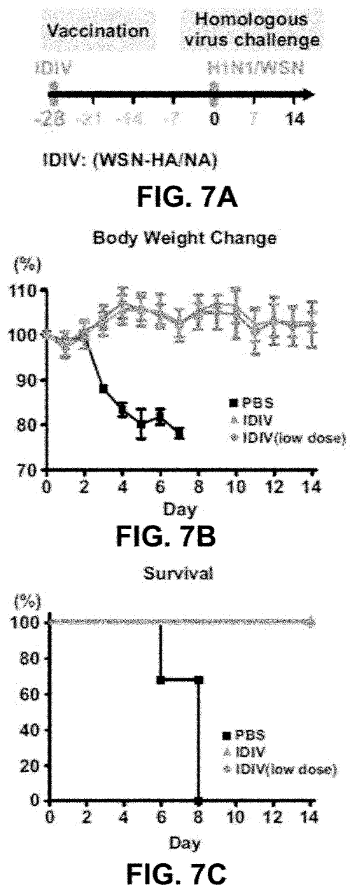

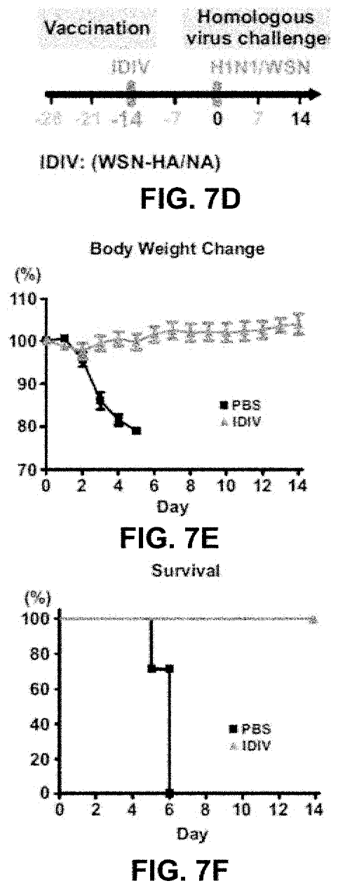

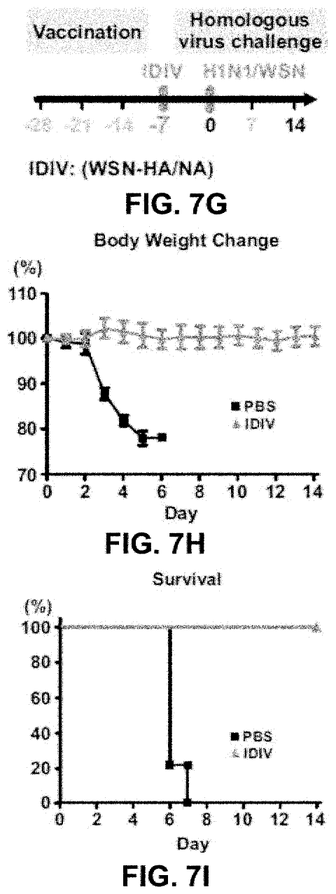

Successful vaccination could protect mice from lethal influenza virus infection at four weeks post-vaccination, which is considered as the full course of immunization. The protection of 28-day IDIV-vaccinated mice against lethal H1N1 infection was therefore tested. As expected, 28-day vaccinated mice groups immunized with low-dose (2×10⁶ PFU) or high-dose (2×10⁸ PFU) of IDIV were fully protected (FIGS. 7A-7C). In addition, no weight loss of mice in both groups was observed after infection (FIG. 7B). As IDIV induced expedited seroconversion, (FIGS. 5C-5D) it was then investigated if it could protect mice at an earlier time post-vaccination. Similar to 28-day immunization, mice vaccinated for 14 days (FIGS. 7D-7F) and 7 days (FIGS. 7G-7I) were all protected from lethal challenge and showed no decrease in body weight nor disease signs.

### IDIV and IDIV-2 Elicits Heterologous Influenza Protection Against H5N1 and H7N9

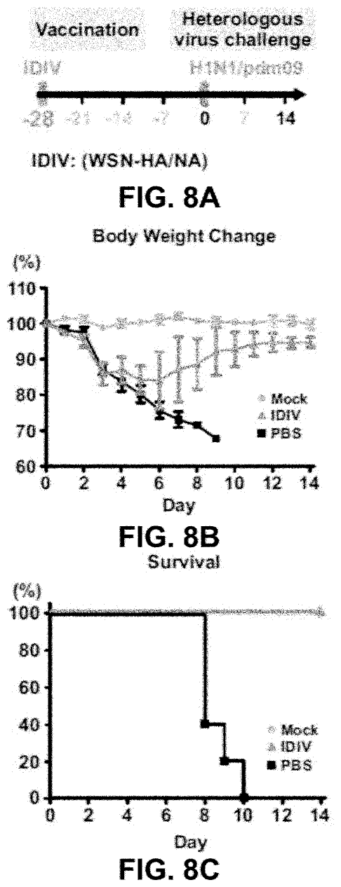

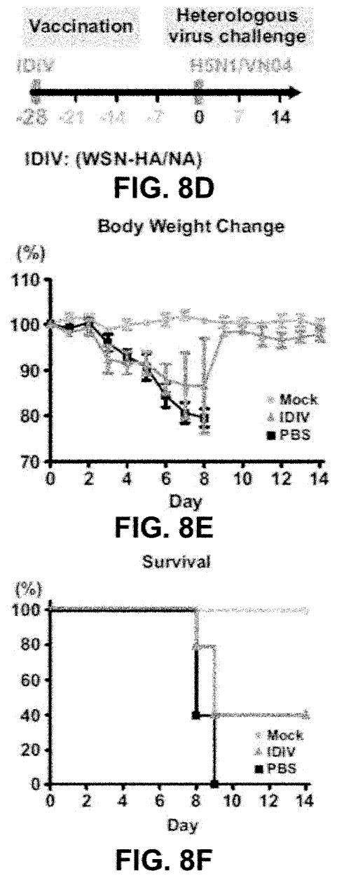

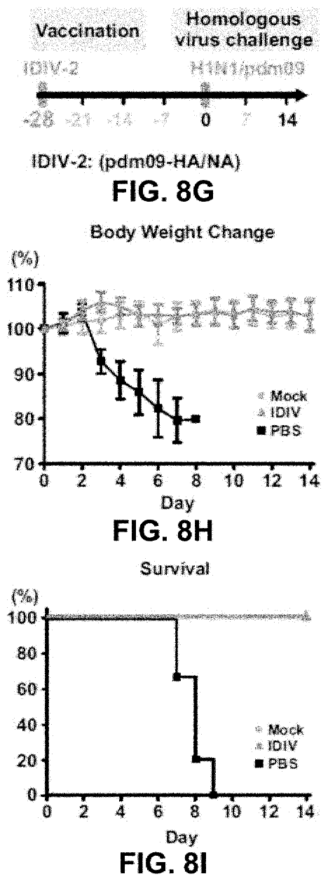

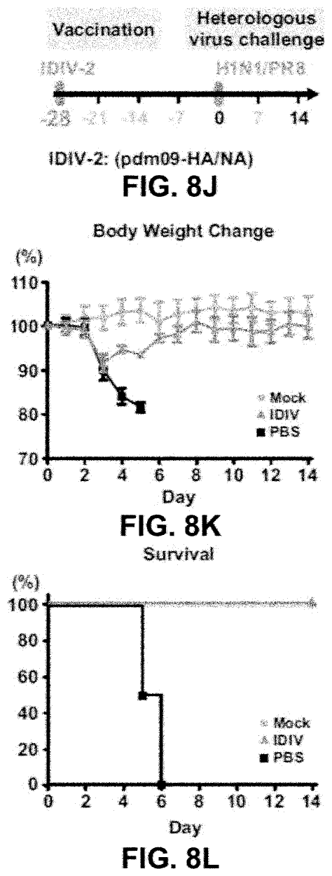

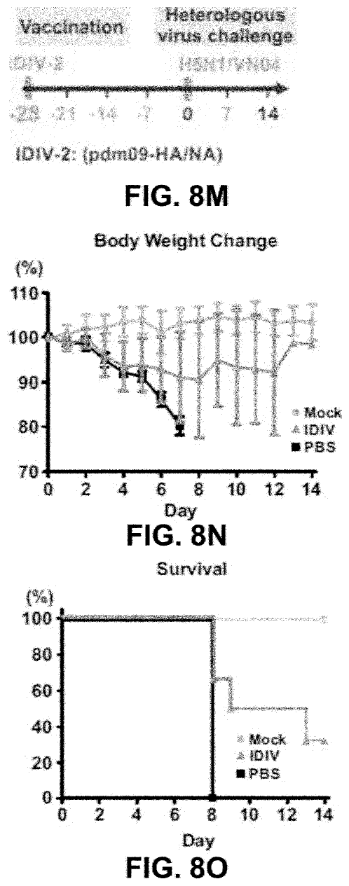

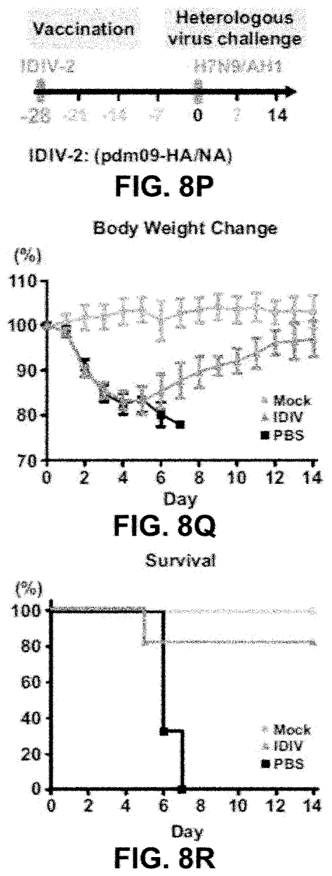

Despite the fact current influenza vaccines can provide reasonable level of protection, their efficacy drops significantly when there is a mismatch between the vaccine strain and the circulating strain. It is therefore desirable that next-generation influenza vaccines can offer cross-protection against heterologous strains of influenza. The IDIV in the current study contained DI viruses with HA and NA from H1N1/WSN strain (FIG. 8A). When 28-day IDIV-vaccinated mice were lethally challenged with the heterologous H1N1/pdm09 virus, all mice were protected, contrary to 100% death in PBS-vaccinated group (FIG. 8A). It is noteworthy that IDIV-vaccinated mice initially showed body weight loss similar to PBS-vaccinated mice (FIG. 8B). But the decrease eased off starting from day 4 post-infection; while the disease of PBS group continued to worsen. The protection against the highly pathogenic H5N1 virus was also tested (FIGS. 8D-8F). IDIV could also partially protect mice from lethal challenge of H5N1 virus, indicating protection across subtypes. In addition to IDIV with WSN-HA and NA, another version of IDIV was also tested, named IDIV-2, which contains HA and NA from H1N1/pdm09 (FIGS. 8G-8R). Similar to IDIV vaccination, IDIV-2-vacinated mice were all protected from lethal infection of homologous H1N1/pdm09 virus without showing weight loss (FIGS. 8G-8I). Vaccinated mice were also fully protected from lethal challenge of heterologous H1N1/PR8 (FIGS. 8J-8L), and partially protected from H5N1/VN04 (FIGS. 8M-8O) and H7N9/AH1 lethal challenge (FIGS. 8P-8R), indicating that IDIV vaccination could offer protection across different influenza subtypes.

### IDIV Vaccination in Mice Induces Potent Cytokine and Chemokine Production

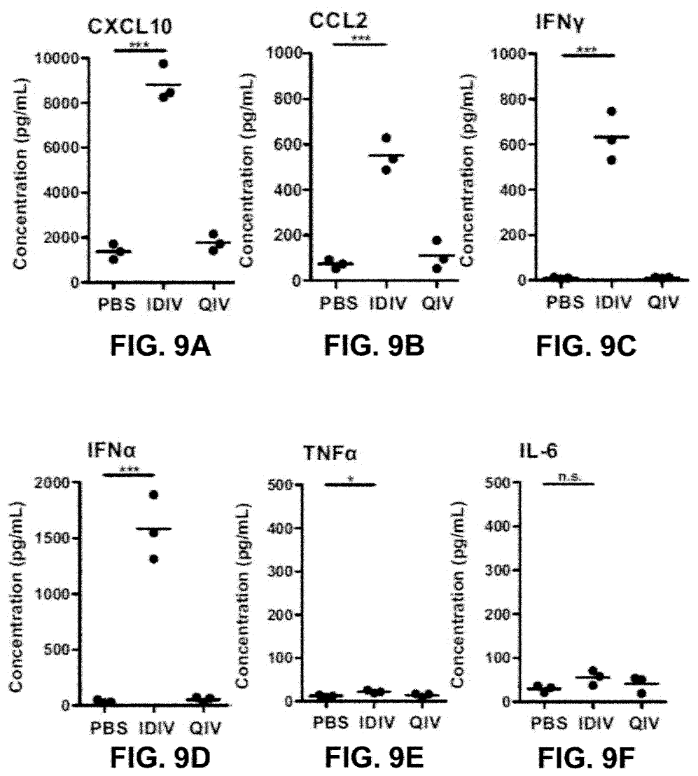

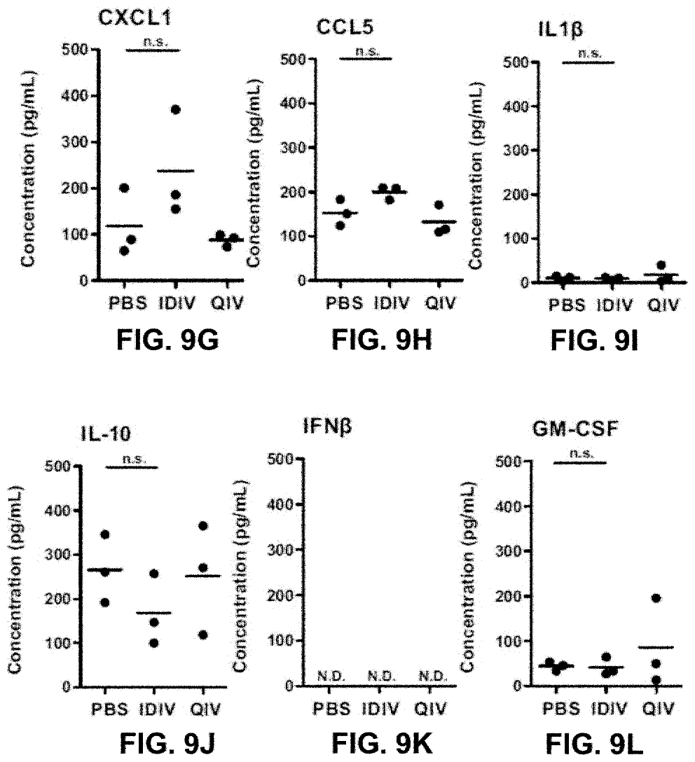

Cytokines/chemokines are important mediators for immune cell recruitment, migration and maturation. In FIGS. 4A-4J, it was demonstrated that in vitro infection of the live-but-defective IDIV robustly induced secretion of chemokine (CXCL10) and type-I/III interferons (IFNB and IFNL1) in human PBDM and MoDC. However, it is not known whether the innate immune response will be activated upon single dose IDIV administration. To understand the early cytokine response after vaccination, sera of vaccinated mice were collected 18 hr post-vaccination for profiling of 12 cytokines/chemokines. In the IDIV vaccinated mice, a subset of chemokines including CXCL10 and CCL2 (MCP-1) was significantly upregulated (FIGS. 9A-9B). CXCL1 and CCL5 also showed a trend of increase, although the induction was statistically insignificant. Moreover, interferon alpha and gamma, which are important cytokines for monocyte activation, were also highly upregulated. Unexpectedly, despite the extraordinary induction of cytokine subsets described above, IDIV did not cause overwhelming systemic inflammation. No increase in pro-inflammatory cytokines IL6 and IL1β were detected in the serum, and only a slight increase in basal level of TNFα was observed (FIGS. 9E, 9F and 9I). Taken together, the induction of cytokines/chemokines may help for the monocyte differentiation, APC activation, and migration; while the absence of severe inflammation is important not to induce adverse side effect.

### IDIV Elicits Adaptive Immune Responses is Independent of Type-I Interferon Signaling

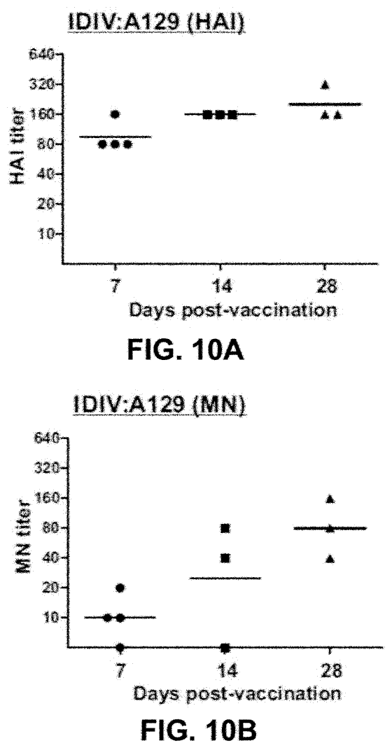

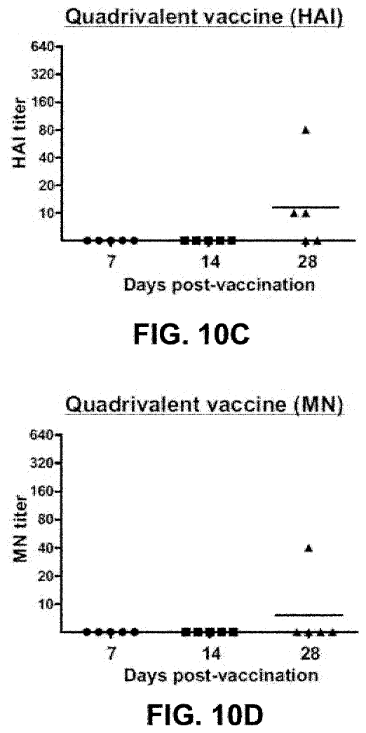

In view of the escalated serum interferon alpha level, it was queried if type I interferon signaling is essential for the optimal development of antibody response upon IDIV vaccination. Therefore, interferon-alpha receptor knockout mouse (Ifnar^{-/-} A129), of which interferon signaling is defective, was used to determine the dependence of type-I interferon signaling. Ifnar^{-/-} A129 mice were vaccinated by IDIV as previously described and sera were collected on days 7, 14, and 28 post-vaccination for quantitation of neutralizing antibody. Nevertheless, considerable amount of neutralizing antibody could still be detected with blunted type I interferon signaling (FIGS. 10A-10B), suggesting that type I interferon signaling is dispensable. This is in line with previous reports that although TLR agonists can boost anti-influenza B cell response, TLR signaling is dispensable for robust antibody response (Heer, et al., J Immunol 178, 2182-2191 (2007); Nemazee, et al., Nature 441, E4; discussion E4 (2006)).

### Discussion

In this study, it has been demonstrated rational design, synthesis, and characterization of an ultra-effective intradermal flu vaccine IDIV. This is the first report that investigated the potential use of single-round infectious virus as intradermal influenza vaccine. Using the regimen described plus intradermal vaccination, single-dose IDIV elicited unprecedented humoral response even without adjuvants. This study provides a rational selection pipeline for a prototypic single-round infectious virus that has optimal growth. After observing ubiquitous presence of truncated genome in clinal samples, a panel of chimeric viruses was constructed by reverse genetics and showed that polymerase genes crucially affect the efficiency of defective virus production (FIG. 1). Furthermore, after SMRT sequencing of the H7-3P virus stock, four most abundantly expressed defective genome sequences were selected for single-round infectious virus production, and finally selected one as the IDIV prototype. The same pipeline could be applied for production of other HA subtype IDIV. And most importantly, this study presented a novel influenza vaccination strategy using the prototypic single-round infectious virus and demonstrated its exceptional performance.

Current influenza vaccines heavily rely on production in chicken embryonated eggs. The cost is low, but egg supply is a limitation that impedes production. Therefore, vaccine production in FDA-approved virus-producing cell-lines has been proposed as an alternative, as exemplified by the commercially available cell-based influenza vaccines (e.g., Flucelvax Quadrivalent) that were approved by US FDA in 2017. One of the limitations during the initial stage of this study is the production yield of IDIV. However, with careful selection of genome sequences and the use of polymerases with strong activity, the yield of recombinant IDIV can now reach 2.5×10⁹ PFU/ml. Virus cultured in one T-75 flask (10 mL of virus culture medium) is enough for production of two high-dose (2×10⁸ PFU) or equivalently two hundred low-dose (2×10⁶ PFU) IDIV used in this study.

Apart from increasing the amount of antigens delivered, a vaccine formulation that could adequately activate innate immune response was also investigated. It was confirmed that IDIV was able to potently stimulate innate immune response in human PBDMs and MoDCs (FIGS. 4A-4J). Live vaccines have been speculated to have superior performance over inactivated vaccines. The reason, in part, is that molecules including antigens and the natural immunostimulatory components in live vaccines are preserved in their native forms; and the vaccination can mimic a real viral infection. Because the IDIV is completely non-replicative, it allows IDIV to be administered safe enough without inactivation. The intradermal inoculation also minimizes any chance of recombination with any influenza virus present in lung of vaccinee. LAIV currently in use is composed of cold-adapted temperature-sensitive live viruses. However, there might still be concerns about viral replication in immunocompromised vaccinees. The IDIV can serve as an alternative live-but-safe influenza vaccine that is suitable for broader range of people with various immune status.

In addition, this study provided valuable information about the characteristics of immune response upon an intradermal influenza vaccination. Here it has been shown that IDIV induced robust neutralizing antibody production as early as 7 days post-vaccination in mice (FIGS. 5C and 5D), the HAI titer can reach as high as 1:640 at 28 days post-vaccination (FIG. 5A). Among the antibody subclasses assayed, IDIV induced exclusively IgG2a (FIGS. 6A-6C). This was in line with previous report that described the importance of IgG2a in anti-influenza immunity (Huber, et al., Clin Vaccine Immunol 13, 981-990 (2006)). However, the study does not exclude the importance of IgA, which also takes part in mucosal immune defense. Adjuvants are sometimes added to vaccine to boost the immune activation. Fundamentally, these chemicals may promote inflammation, which can cause side-effects both locally (e.g., redness, itch and pain) and systemically (e.g., headache and fatigue). In this study, the self-adjuvanted IDIV did not cause severe inflammation. Only a subset of chemokines (e.g., CXCL10 and CCL2) and interferons (e.g., IFNα and IFNγ) were markedly up-regulated. Surprisingly, inflammatory cytokines such as IL6, 1L1β, and TNFα were not induced at all (FIGS. 9E, 9F, and 9I). And yet, a good humoral response was mounted. This implied that instead of boosting the immune system indiscriminately, activating certain pathway(s) in the immune system may already be enough to promote vaccine efficacy. Further elucidation may help in the design of improved adjuvants that have enhanced specificity and effectiveness but cause fewer side-effects.

It was also demonstrated that a single dose IDIV was enough to elicit heterologous protection against H5N1 and H7N9 (FIGS. 8M-8R). A few aspects regarding the mode of protection still await further studies. Since a non-replicative influenza vaccine hardly induces T-cell response due to the lack of viral protein expression in cells, the protection against H5N1 and H7N9 lethal infection may not be solely due to the involvement of cytotoxic T lymphocytes. One possibility may be that IDIV induced production of broadly neutralizing antibodies (bnAbs), for example antibodies against the more conserved stalk region. The characterization of APC activation, presence of bnAbs and induction of T cell response upon IDIV immunization are worth investigating.

In summary, combining the use of a live, self-adjuvanted, but completely replication-defective virus, together with antigen delivery in increased amount and intradermal route of administration, IDIV achieved incomparable vaccination efficacy and elicited heterologous influenza protection.

### Example 2: Strategy for Efficient Production of Defective Interfering (DI) Virus

Defective interfering (DI) virus lacks parts of the genome essential for replication and is characterized by its ability to interfere with the replication of standard virus. Despite being present across different strains of influenza virus, isolation of a substantial amount of pure DI virus from standard virus remains one of the biggest obstacle in the application of DI. A platform for efficient production of pure DI virus by reverse genetics is described.

### Methods

### Cell-Lines

MDCK cells and HEK293FT cells were obtained from American Type Culture Collection and Thermo Fisher Scientific respectively. MDCK cells were cultured in Minimal Essential Medium (MEM) (Thermo Fisher Scientific). MDCK-SIAT1 cells and HEK293FT cells were maintained in Dulbecco's Modified Eagle Medium (DMEM) (Thermo Fisher Scientific), all supplemented with 10% Fetal bovine serum (FBS) (Thermo Fisher Scientific). Cells were kept at 37° C. with 5% CO₂. MDCK-PB2, MDCK-SIAT1-PB2 and 293 FT-PB2 stable cells were generated by transduction of lentivirus carrying a H7N9 (A/Zhejiang/01/2013) PB2 transgene, followed by 2 μg/mL puromycin (Thermo Fisher Scientific) selection. To generate MDCK-2P and 293FT-2P stable cells, MDCK-PB2 and 293 FT-PB2 were subsequently transduced by lentivirus carrying a H7N9 (A/Zhejiang/01/2013) PB1 transgene, followed by 150 μg/mL hygromycin B (Thermo Fisher Scientific) selection. To generate MDCK-3P, MDCK-2P cells were transduced by lentivirus carrying a Tet-ON 3G activator transgene, followed by 7 μg/mL blasticidin (Thermo Fisher Scientific) selection. The selected cells were then transduced by lentivirus carrying a PA transgene driven by tetracycline responsive element (TRE) 3G promoter. The transduced cells were selected by 800 μg/mL geneticin (Thermo Fisher Scientific).

### Generation of Defective Interfering (DI) Virus

To generate recombinant DI viruses, reverse genetics was performed in a co-culture of MDCK stable cells and 293FT stable cells. The 8 plasmid system was used for reverse genetics as previously described. The DI segment was cloned into a pPoll expression plasmid which is driven by RNA polymerase I promoter (pPoll). The remaining H7N9 segments were expressed by bi-directional expression plasmids and the H1N1 or H3N2 segments were expressed by pPoll expression plasmids or bi-directional expression plasmids. The transfected cells were incubated for 72 hours in plain MEM supplemented with TPCK-trypsin before the supernatant was passaged to fresh MDCK stable cells for propagation. Plaque assay was performed in MDCK stable cells and parental MDCK cells to examine the titer of the DI virus and to confirm the absence of replicative virus respectively. The DI virus can be further purified by 25% sucrose-cushioned ultracentrifugation at 28,000 rpm for 4 hours after passing through 0.45 μm filters.

### Production of Lentivirus

To package lentivirus, 293FT cells were transfected with 3 μg of lentiviral transfer plasmid and 9 μg of ViralPower Lentivirus Packaging Mix (Thermo Fisher Scientific). 48 hours after transfection, the lentivirus-containing medium was centrifuged at 4,000×g for 3 minutes and then filtered through a 0.45 μm filter (Millipore), subsequently precipitated by PEG-it (System Bioscience) solution. The lentivirus was stored at −80° C.

### RT-PCR

Viral RNA from supernatant was extracted using QIAamp® Viral RNA Mini Kit (Qiagen). Reverse transcription of the viral RNA was performed with PrimeScript RT reagent kit with gDNA eraser (Takara) using primer 5′-AGCAAAAGCAGG-3′ (SEQ ID NO: 32). PCR of viral segments were carried out using DreamTaq Green DNA Polymerase (Thermo Fisher Scientific) with segment-specific primers.

### Plaque Assay

Plaque assay of influenza DI virus was performed in MDCK cells and MDCK stable cells for titer determination. 10-fold dilutions of the virus sample was inoculated onto the cells, which were then overlay by a mixture of 2×MEM and 2% low-melting agarose (Thermo Fisher Scientific) with TPCK-trypsin.

### Sanger Sequencing

For the Sanger sequencing of de novo DI species derived from the H1N1/WSN-DI477 RT-PCR result, the corresponding bands in the DNA agarose gel were excised and the PCR products were ligated to pGEM-T easy plasmid. T7 forward and reverse universal primers were used for sequencing.

### Results

### Design and Packaging of a PB1-DI Virus

A/H7N9 has a strong propensity in generating DI virus, as observed in patient samples and chimeric A/H1N1 virus bearing H7N9 polymerase (Lui, et al. Emerging microbes & infections, 8.1 (2019): 662-674). To establish a reverse genetics platform for efficient influenza DI virus production, a trans-complementation system to harness the unique property of H7N9 polymerase was generated. The platform utilizes virus-producing cell lines stably expressing H7N9 polymerase subunits to propagate DI virus with truncated polymerase segments. DI species which have high compatibility with other viral segments were identified and selected for the packaging of DI virus harboring different influenza antigens. As demonstrated in Example 1, the production of a H1N1/WSN/PB2-322 was achieved using a cell line stably expressing PB2, in which the DI virus carries a truncated PB2 segment with only 322 nucleotides in length.

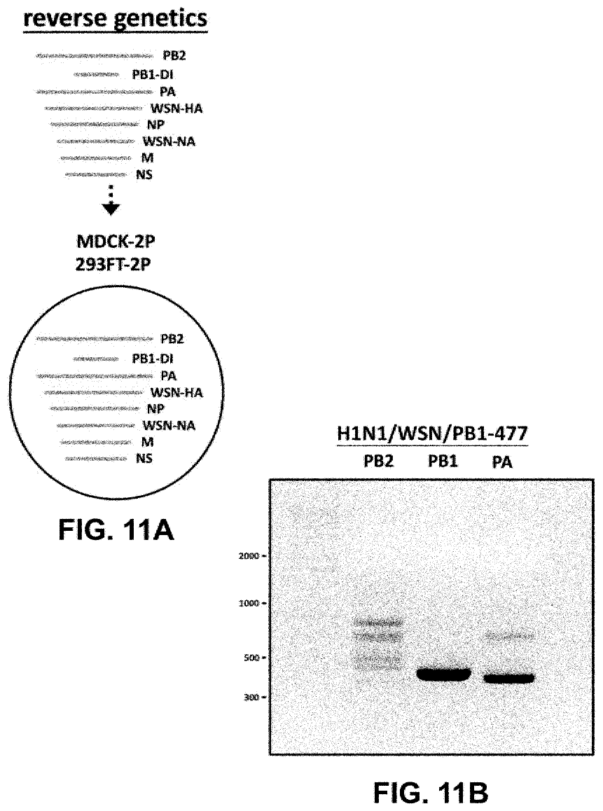

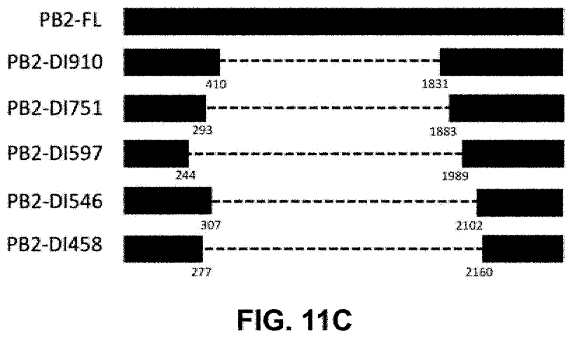

To generate a cell line which supports the replication of the PB1-DI virus, the PB1 transgene was introduced into the PB2 stable cell lines by lentivirus transduction. 13 silent mutations were introduced into 5′ and 3′ ends of the PB1 coding sequence to minimize the chance of recombination with the PB1 DI segment during virus replication. The viral segments PB2, PB1, PA and NP were derived from H7N9, while the segments HA, NA, M and NS were derived from WSN. To package the PB1-DI477 virus, co-transfection of the pPolI-PB1-DI477 plasmid and the plasmids for the remaining viral segments into the co-culture of MDCK-2P and 293FT-2P was performed (FIG. 11A). The reverse genetics product was passaged to fresh MDCK-2P cells in which cytopathic effect was observed. The incorporation of the PB1-DI477 segment into the DI virus was confirmed by RT-PCR (FIG. 11B).

Apart from the PB1-DI477 segment, multiple DI species were present in the PB2 and PA segments as well. Since these newly generated DI species may possess compatibility with the PB1-DI477 sequence, the de novo PB2 DI species were sequenced and cloned into pPoll vector (FIG. 11C).

### Design and Packaging of a PB2 PB1-2DI Virus

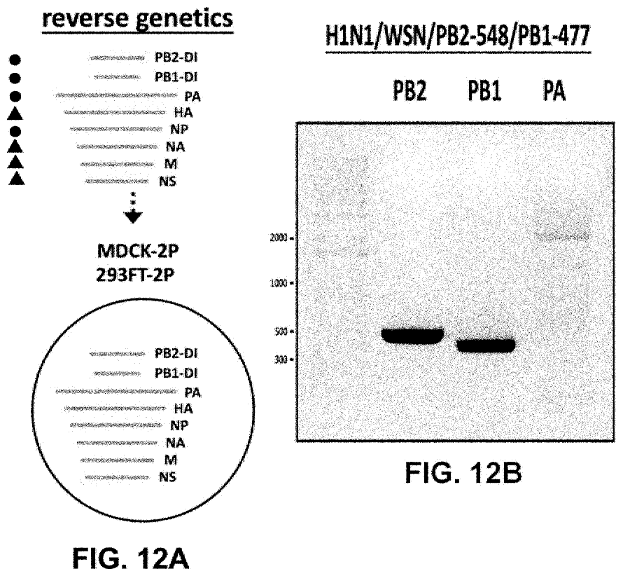

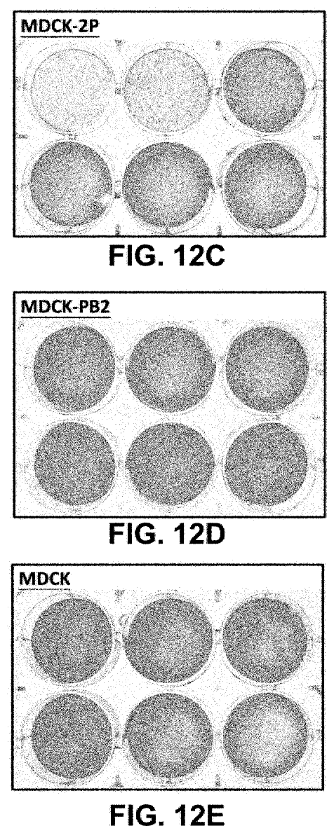

Previous studies on influenza DI viruses mainly focused on virus possessing a single DI segment. DI virus harboring more than one DI segments is theoretically possible, but their presence is yet to be determined due to technical difficulties in isolation of such DI virus. These DI viruses may have an advantage in terms of antiviral activities due to extra copies of DI segments. To package the PB2-PB1 2DI virus, co-transfection of the pPoll-PB2-DI597, pPolI-PB1-DI477 plasmid, and the plasmids for the remaining viral segments into the co-culture of MDCK-2P and 293FT-2P was performed (FIG. 12A). The reverse genetics product was passaged to fresh MDCK-2P cells and the H1N1/WSN/PB2-597/PB1-477 2DI virus exhibited cytopathic effect. The incorporation of both PB2-DI597 and PB1-DI477 segments into the DI virus was confirmed by RT-PCR (FIG. 12B). Plaque assay was performed in MDCK-2P, MDCK-PB2 and MDCK, respectively, to determine the infectivity of the H1N1/WSN/PB2-597/PB1-477 2DI virus. Only MDCK-2P supports the replication of the H1N1/WSN/PB2-597/PB1-477 2DI virus, of which the titer could reach up to 2×10⁶ PFU/mL (FIGS. 12C-12E). The absence of any plaques in MDCK-PB2 and MDCK indicates that the H1N1/WSN/PB2-597/PB1-477 2DI virus requires complementation of both PB2 and PB1 in the cell lines for replication.

### Packaging DI Virus of Different H1N1 Strains

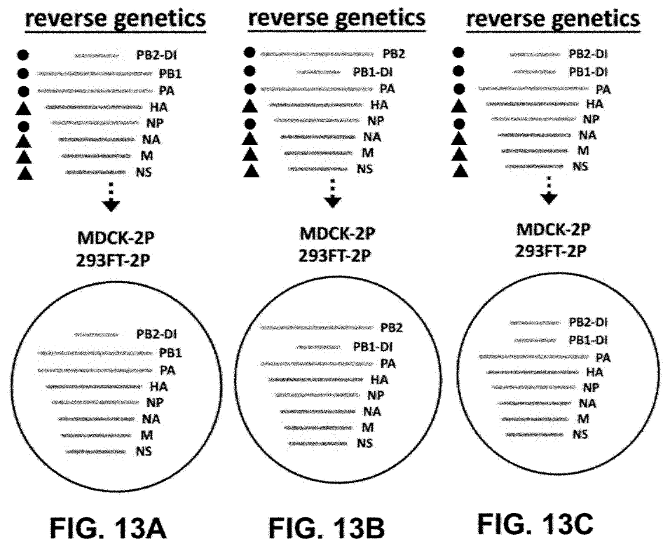

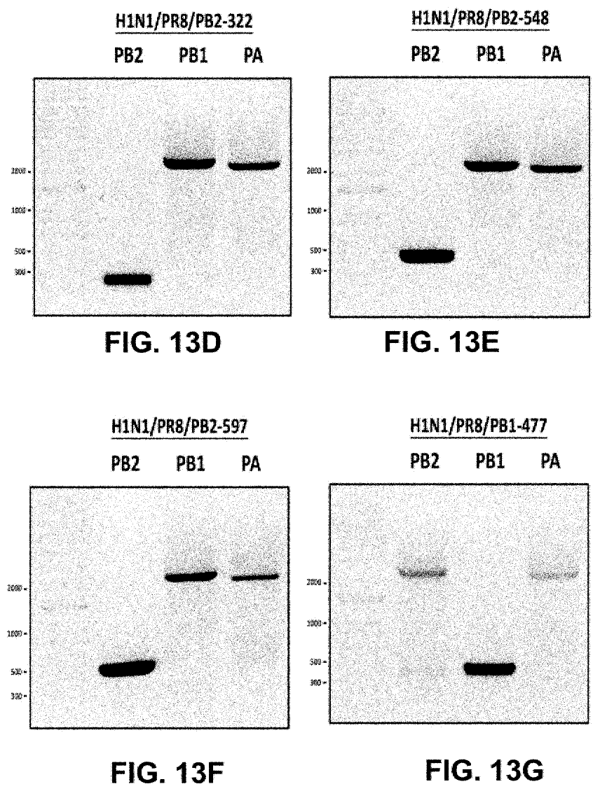

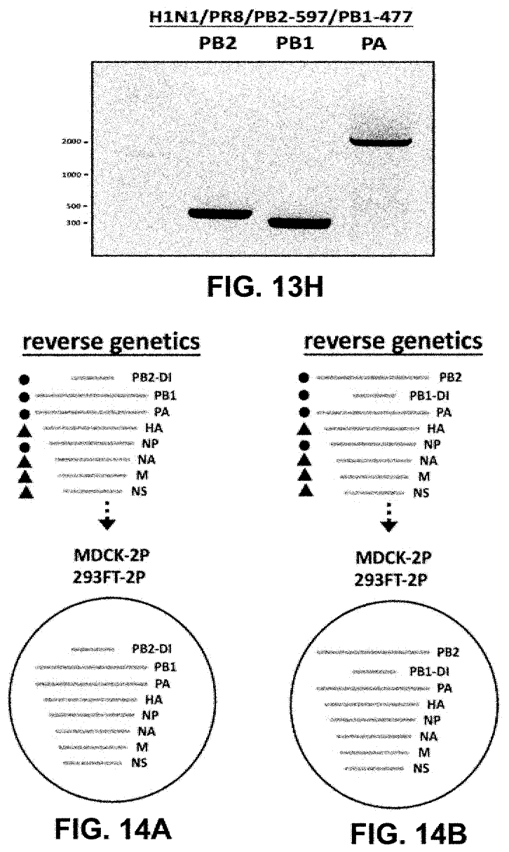

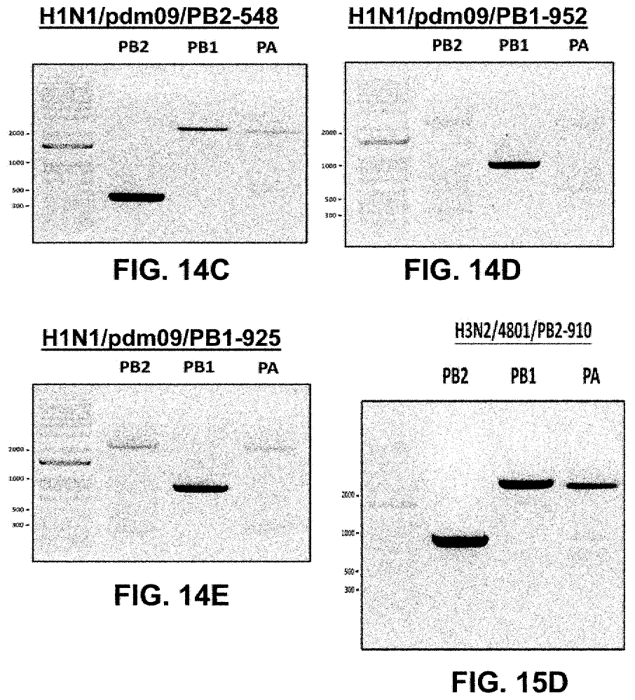

To examine whether this DI virus reverse genetics platform can be widely applied, additional H1N1 DI viruses using another laboratory strain A/PR8/34 (PR8) and a circulating strain A/HK/415742/2009 (pdm09) were generated. For the PR8 DI virus, the viral segments PB2, PB1, PA and NP were derived from H7N9, while the segments HA, NA, M and NS were derived from PR8 (FIGS. 13A, 13B and 13C). Three DI viruses harboring a PB2 DI segment (DI322, DI548 and DI597) were successfully rescued (FIGS. 13D, 13E and 13F), one harboring a PB1 DI segment (DI477) (FIG. 13G) and one harboring both PB2 and PB1 DI segments (DI597 and DI477) (FIG. 13H). For the pdm09 DI virus, the viral segments PB2, PB1, PA and NP were derived from H7N9, while the segments HA, NA, M and NS were derived from pdm09 (FIGS. 14A and 14B). A pdm09 DI virus harboring the PB2-DI548 segment (FIG. 14C) and two harboring a PB1 DI segment (DI952 and DI925) (FIGS. 14D and 14E) were successfully generated. RT-PCR was carried out to confirm the incorporation of the designated DI segments into the DI virus.

### Design and Packaging of H3N2 DI Virus

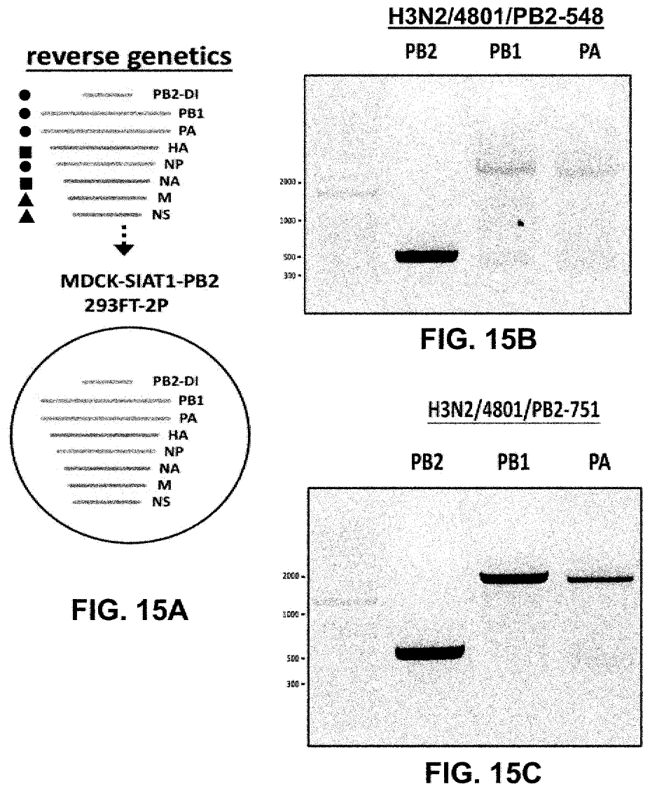

To generate a cell line which supports the replication of the PB2-DI virus with H3N2 antigens, the PB2 transgene was introduced into the MDCK-SIAT1 cells by lentivirus transduction. MDCK-SIAT1 cells, with enhanced expression of α-2,6-linked sialic acid receptor compared to MDCK cells, are superior in preserving the antigenic properties of H3N2 (Lin, et al. Influenza and other respiratory viruses 11.3 (2017): 263-274). 12 silent mutations were introduced into 5′ and 3′ ends of the PB2 coding sequence to minimize the chance of recombination with the PB2 DI segment. The viral segments PB2, PB1, PA and NP were derived from H7N9. The segments HA and NA were derived from H3N2 (Hong Kong/4801/14). The segments M and NS were derived from PR8. To package the H3N2 virus, co-transfection of the pPolI-PB2-DI plasmids and the plasmids for the remaining viral segments into the co-culture of MDCK-SIAT1-PB2 and 293 FT-2P was performed (FIG. 15A). The incorporation of the PB2-DI segment into the DI virus was confirmed by RT-PCR (FIGS. 15B, 15C and 15D). H3N2/4801/PB2-548, H3N2/480/PB2-751 and H3N2/4801/PB2-910 were successfully generated.

### Design and Packaging of PA-DI Virus

To generate a DI virus with a PA DI segment, complementary expression of PA has to be established in the MDCK-2P cell line. To counter the cytotoxic effects of PA, a Tet-ON 3G inducible expression system for PA was designed. The system consists of 2 parts: the Tet-ON 3G trans-activator and the tetracycline responsive element (TRE) 3G promoter-driven PA transgene. Inducible expression of PA can be achieved upon doxycycline treatment. 13 silent mutations were introduced into 5′ and 3′ ends of the PA coding sequence to minimize the chance of recombination with the PA DI segment.

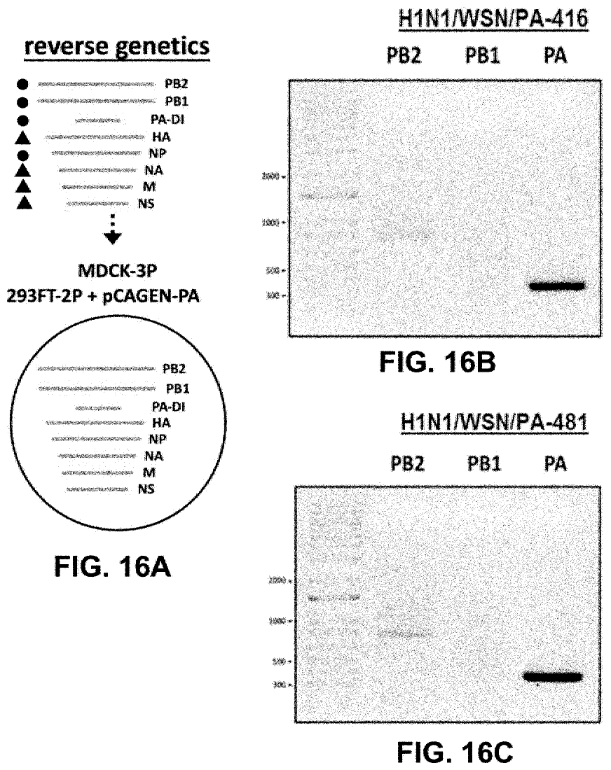

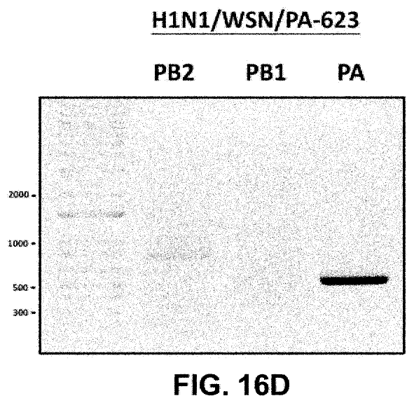

The viral segments PB2, PB1, PA and NP were derived from H7N9, while the segments HA, NA, M and NS were derived from WSN. To package the PA-DI virus, co-transfection of the pPolI-PA-DI plasmid and the plasmids for the remaining viral segments into the co-culture of MDCK-3P and 293FT-2P was carried out (FIG. 16A). An additional pCAGEN-H7-PA was transfected since PA was not stably expressed in the 293FT-2P cell line. Doxycycline was added to the co-culture during medium change 6 hours after transfection to induce the expression of PA in MDCK-3P. The reverse genetics product was passaged to fresh MDCK-3P cells, in which doxycycline was added 6 hours before inoculation. Cytopathic effect was observed and the incorporation of the PA-DI segment into the DI virus was confirmed by RT-PCR (FIGS. 16B, 16C and 16D). H1N1/WSN/PA-416, H1N1/WSN/PA-481 and H1N1/WSN/PA-623 was successfully generated. A summary of the DI virus generated using the platform is presented in Table 1, below.

TABLE 1
Summary of the DI virus generated

| | H1N1 | | | H3N2 |
|---|---|---|---|---|
| | PR8 | WSN | pdm09 | 4801 |
| Single DI | PB2-322 | PB1-477 | PB2-548 | PB2-548 |
| | PB2-548 | PA-416 | PB1-925 | PB2-751 |
| | PB2-597 | PA-481 | PB1-952 | PB2-910 |
| | PB1-477 | PA-623 | | |
| 2DI | PB2-597/PB1-477 | PB2-597/PB1-477 | | |

### Discussion

In this study, a cell-based trans-complementation system for the reverse genetics of DI virus was generated. Trans-complementation system for pure influenza DI virus production has been reported in recent years, albeit limited to segment one (Yamagata, et al., Microbiology and immunology 63.5 (2019): 164-171; and Bdeir, et al. PLOS One 14.3 (2019): e0212757). DI RNA from segments one to three can be efficiently packaged into virions to generate different desired DI virus species, allowing study the biology of such DI virus. DI virus harboring DI RNA from both segments one and two is demonstrated. It has also been shown that the surface antigens of the DI virus can be substituted with different strains while retaining the same genomic backbone, allowing us to rapidly generate vaccine candidates of the desired strains.

This study also explored the compatibility of DI RNA with other viral segments. Packaging of intracellular DI species into progeny virions is not a random process. It has been suggested that different factors such as the sufficient retention of packaging signals play a role in determining the packaging efficiency of DI RNA (Alnaji, et al. Mbio 12.6 (2021): e02959-21). It can be observed that PB2-DI322 can be incorporated alongside various H1N1 segments. H3N2/4801, on the other hand, does not readily incorporate the PB2-DI322 segment. Meanwhile, PB2-DI548 exhibits compatibility with both H1N1/PR8 and H3N2/4801. While PB1-DI477 is compatible with both H1N1/WSN and H1N1/PR8, the virus could not be rescued using H1N1/pdm09 segments. These results point out that packaging of DI RNA is not necessarily a stochastic process and natural selection may exist for the best-fitting DI RNA. The intrinsic properties of the DIRNA sequence may determine its packaging advantage.

Differential compatibility among DI species from different segments is still poorly understood. DI virus with more than one DI genome theoretically exist in nature. However, identifying a combination of DI species from different segments that are compatible to one another remains challenging. In this study, a sequential approach was used to address this question. From a PB1-DI virus, cognate PB2-DI species were identified which are abundant in amount, indicating their replicative advantage in the presence of PB1-DI RNA. The 2DI virus was eventually rescued by reverse genetics, which can reach up to 2×10⁶ PFU/mL. The high titer suggested that the PB2-DI and PB1-DI species can be efficiently propagated and packaged together. This highlights the potential of SMRT sequencing to identify the most abundant DI species within the virus mixture since it may possess the highest selective advantage to be packaged and propagated, hence the highest chance to be reproduced at high titer by reverse genetics in the trans-complementation system. Coupling the use of long-read sequencing and trans-complementary cell lines greatly benefits the study of DI virus.

The generation of a DI virus with two DI RNA from segments one and two is a breakthrough. The resultant 2DI virus H1N1/WSN/PB2-597/PB1-477 can be propagated at a relatively high titer. This reagent would be an interesting candidate in exploring the antiviral potential of using DI virus against influenza infection. The interfering and immunostimulatory property of DI virus provide considerable potential to be used as a therapeutic agent. Compared to conventional DI virus bearing a single DI RNA in its genome, the extra DI RNA may augment both its interfering and immunostimulatory effect. It also increases safety in a clinical setting and reduces the chance of reassortment of full-length polymerase segments with standard virus. A recent study using a DI virus with two DI RNA derived from segments one and three suggested that an extra DI RNA does not enhance its antiviral activity (Bdeir, et al., Scientific reports 11.1 (2021): 1-10). The DI RNA used in the study, however, are roughly one thousand base pairs in length. The significantly longer DI RNA may not be as effective as shorter DI RNA in attenuating standard virus replication. In addition, the DI virus only reaches a titer of 10⁴ ffu/mL, implying that the DI RNA selected may not be preferentially packaged and incorporated into progeny virions, limiting its antiviral potential. Hence, the advances achieved in the production of 2DI may hint at a different experimental outcome. With the trans-complementation of all three subunits of the viral polymerase in the described cell culture system, it is possible to generate a DI virus harboring three DI RNA from segments one to three, fully utilizing the antiviral potential of DI virus.

The establishment of a cell-culture based DI virus system also serves as a rapid vaccine candidate manufacturing platform. Egg-based inactivated vaccine remains one of the major influenza vaccines available. Despite having the merits of low production cost and rapid manufacturing, the limited immune response elicited upon administration remains one of the major disadvantages (He, et al., J Virol 80, 11756-11766 (2006)). The robust humoral immunity elicited by DI virus renders it an edge over inactivated vaccines. Annual epidemics are generally caused by H1N1 and H3N2. The results demonstrated that circulating strains of H1N1 and H3N2 can be readily applied to our system, showing its translational potential to the clinical setting. The data have demonstrated that the trans-complementation cell line platform and rational approach on sequence compatibility allows for the systematic generation of various kinds of DI virus, shedding light on the dynamic potential on future application.

Those skilled in the art will recognize, or be able to ascertain using no more than routine experimentation, many equivalents to the specific embodiments of the method and compositions described herein. Such equivalents are intended to be encompassed by the following claims.

1. An intact, replication-deficient virus including an influenza virus genome, wherein the virus infects normal human cells,
wherein the influenza virus genome includes one or more mutations in one or more genes selected from the group including viral RNA polymerase PB1, viral RNA polymerase PB2, viral RNA polymerase PA, and nucleoprotein (NP), and
wherein the one or more mutations prevents or reduces replication of the virus in normal human cells by at least 90% as compared to the same virus in the absence of the mutation,
optionally wherein the virus is 100% non-replicating in normal mammalian cells.

2-3. (canceled)

4. The replication-deficient virus of claim 1, wherein the influenza virus genome includes one or more mutations in the viral RNA polymerase PB2 gene that prevents or reduces replication of the virus in normal human cells,
optionally wherein the influenza virus genome includes a viral RNA polymerase PB2 gene having the nucleic acid sequence of any one of SEQ ID NOs: 1 or 9-15, or a nucleic acid sequence having at least 75% identity to any one of SEQ ID NOs: 1 or 9-15.

5-12. (canceled)

13. The replication-deficient virus of claim 1, wherein the influenza virus genome includes one or more mutations in the viral RNA polymerase PA gene that prevents or reduces replication of the virus in normal human cells,
optionally wherein the influenza virus genome includes a viral RNA polymerase PA gene having the nucleic acid sequence of any one of SEQ ID NOs: 16-19, or a nucleic acid sequence having at least 75% identity to any one of SEQ ID NOs: 16-19.

14-17. (canceled)

18. The replication-deficient virus of claim 1, wherein the influenza virus genome includes one or more mutations in the viral RNA polymerase PB1 gene that prevents or reduces replication of the virus in normal human cells,
optionally wherein the influenza virus genome includes a viral RNA polymerase PB1 gene having the nucleic acid sequence of any one of SEQ ID NOs: 20-27, or a nucleic acid sequence having at least 75% identity to any one of SEQ ID NOs: 20-27.

19-26. (canceled)

27. The replication-deficient virus of claim 1, wherein the influenza virus genome includes eight genomic segments,
wherein between one and seven of the genomic segments are derived from a first influenza virus, and
wherein between one and seven of the genomic segments are derived from a second influenza virus, and
wherein the one or more mutations that prevent or reduce viral replication are present in the genomic segments derived from the second virus.

28. The replication-deficient virus of claim 27, wherein the genome includes between five and seven genomic segments of the first influenza virus, and
wherein the first virus is a replication-competent influenza A virus selected from the group including H1, H2, H3, H5, H6, H7, H9, and H10 subtypes; and/or
wherein the first virus is selected from the group including N1, N2, N6, N7, N8 and N9 subtypes.

29. (canceled)

30. The intact, non-replicating virus of claim 27, wherein the second virus is an influenza virus selected from the group including H1, H2, H3, H5, H6, H7, H9, and H10 subtypes; and/or
wherein the second virus is selected from the group including N1, N2, N6, N7, N8 and N9 subtypes.

31. (canceled)

32. An intact, replication-deficient virus including an influenza virus genome,
wherein the virus infects normal human cells,
wherein the influenza virus genome includes eight genomic segments,
wherein between one and seven of the genomic segments are derived from a first influenza virus,
wherein between one and seven of the genomic segments are derived from a second influenza virus,
wherein the influenza virus genome includes one segment including a viral RNA polymerase PB1 gene and one segment including a viral RNA polymerase PB2 gene,
wherein the influenza virus genome includes one or more mutations in the viral RNA polymerase PB1 gene and one or more mutations in the viral RNA polymerase PB2 gene, and
wherein the one or more mutations prevent or reduce replication of the virus in normal human cells by at least 90% as compared to the same virus in the absence of the mutation,
optionally wherein the virus includes one or more exogenous genes derived from a defective-interfering (DI) particle.

33. The replication-deficient virus of claim 32, wherein the viral RNA polymerase PB1 gene has the nucleic acid sequence of any one of SEQ ID NOs: 20-27, or a nucleic acid sequence having at least 75% identity to any one of SEQ ID NOs: 20-27.

34-40. (canceled)

41. The replication-deficient virus of claim 32, wherein the viral RNA polymerase PB2 gene has the nucleic acid sequence of any one of SEQ ID NOs: 1 or 9-15, or a nucleic acid sequence having at least 75% identity to any one of SEQ ID NOs: 1 or 9-15.

42-48. (canceled)

49. The intact, non-replicating virus of claim 32, wherein the first or second influenza virus is a replication-competent influenza A virus selected from the group including H1, H2, H3, H5, H6, H7, H9, and H10 subtypes, and/or
wherein the first or second virus is selected from the group including N1, N2, N6, N7, N8 and N9 subtypes.

50-52. (canceled)

53. The intact, non-replicating virus of claim 32, wherein the influenza virus genome further comprises one or more mutations in the one or more genes selected from viral RNA polymerase PA, and nucleoprotein (NP),
optionally wherein the influenza virus genome includes a viral RNA polymerase PA gene having the nucleic acid sequence any one of SEQ ID NOs: 16-19, or a nucleic acid sequence having at least 75% identity to any one of SEQ ID NOs: 16-19.

54-57. (canceled)

58. The intact, non-replicating virus of claim 32, wherein the genome includes between five and seven genomic segments of the first influenza virus,
optionally wherein the first virus is 100% non-replicating in normal mammalian cells.

59. The intact, non-replicating virus of claim 32, wherein the first and second viruses are of different subtypes, or are different strains of the same subtype.

60-61. (canceled)

62. The replication-deficient virus of claim 32, wherein the first or second influenza virus is selected from the group including H1N1, H3N1, H5N1, H7N9 and H2N2 subtypes,
optionally wherein the first and/or second influenza virus is selected from the group consisting of A/WSN/1933 (H1N1), A/PR8/34 (H1N1), A/HK/415742/2009 (H1N1), and A/HK/4801/2014 (H3N2).

63-66. (canceled)

67. The replication-deficient virus of claim 32, wherein the genome includes between one and three genomic segments derived from the second influenza virus, and
wherein the second virus is
(a) H5N1 subtype; or
(b) H7N9 subtype,
optionally wherein the genome includes a mutated PB1, PB2 and/or PA gene derived from an H7N9 subtype virus.

68-69. (canceled)

70. An intact, non-replicating virus including an influenza virus genome,
wherein the virus infects normal human cells,
wherein the virus is completely non-replicating in normal human cells, and
wherein the influenza virus genome comprises
(A)
(i) genomic segments 4-8 of the A/WSN/1933 (H1N1) genome having a nucleic acid sequence of SEQ ID NOs: 4-8; and
(ii) genomic segment 1 including a mutated PB2 gene having a nucleic acid sequence of any one of SEQ ID NOs: 1 or 9-15, or a nucleic acid sequence having at least 75% identity to any one of SEQ ID NO:1 or 9-15; and
(iii) genomic segments 2 and 3 including the PB1 and PA genes of an H7N9 virus having a nucleic acid sequence of SEQ ID NOs: 2 and 3, or
(B)
(i) genomic segments 4 and 6 of the A/HK/4801/2014 (H3N2) genome; and
(ii) genomic segment 1 including a mutated PB2 gene having a nucleic acid sequence of SEQ ID NO:11, 13 or 15, or a nucleic acid sequence having at least 75% identity to SEQ ID NO 11, 13 or 15, and
(iii) genomic segments 2 and 3 including the PB1 and PA genes of an H7N9 virus having a nucleic acid sequence of SEQ ID NOs: 2 and 3, and
(iv) optionally wherein the influenza virus genome comprises genomic segments 7 and 8 from A/PR8/34 (H1N1), or
(C)
(i) genomic segments 4 and 6-8 of the A/HK/415742/2009 (H1N1) genome; and
(ii) genomic segment 1 including a mutated PB2 gene having a nucleic acid sequence of SEQ ID NO: 13, or a nucleic acid sequence having at least 75% identity to SEQ ID NO:13, and
(iii) genomic segments 2 and 3 including the PB1 and PA genes of an H7N9 virus having a nucleic acid sequence of SEQ ID NOs: 2 and 3, or
(D)
(i) genomic segments 4 and 6-8 of the A/HK/415742/2009 (H1N1) genome; and
(ii) genomic segment 2 including a mutated PB1 gene having a nucleic acid sequence of SEQ ID NO:26 or 27, or a nucleic acid sequence having at least 75% identity to SEQ ID NO:26 or 27, and
(iii) genomic segments 1 and 3 including the PB2 and PA genes of an H7N9 virus, or
(E)
(i) genomic segments 4 and 6-8 of the A/PR8/34 (H1N1) genome; and
(ii) genomic segment 2 including a mutated PB1 gene having a nucleic acid sequence of SEQ ID NO:22, or a nucleic acid sequence having at least 75% identity to SEQ ID NO:22,
(iii) genomic segment 1 including a mutated PB2 gene having a nucleic acid sequence of SEQ ID NO:14, or a nucleic acid sequence having at least 75% identity to SEQ ID NO:14, and
(iv) genomic segment 3 including the PA gene of an H7N9 virus having a nucleic acid sequence of SEQ ID NO:3, or
(F)
(i) genomic segments 4 and 6-8 of the A/PR8/34 (H1N1) genome; and
(ii) genomic segment 1 including a mutated PB1 gene having a nucleic acid sequence of SEQ ID NO:1, or a nucleic acid sequence having at least 75% identity to SEQ ID NO:1, and
(iii) genomic segments 2 and 3 including the PB2 and PA genes of an H7N9 virus having a nucleic acid sequence of SEQ ID NOs: 2 and 3, respectively, or
(G)
(i) genomic segments 4 and 6-8 of the A/PR8/34 (H1N1) genome; and
(ii) genomic segment 2 including a mutated PB1 gene having a nucleic acid sequence of SEQ ID NO:22, or a nucleic acid sequence having at least 75% identity to SEQ ID NO:22,
(iii) genomic segments 1 and 3 including the PA gene of an H7N9 virus, or
(H)
(i) genomic segments 4-8 of the A/WSN/1933 (H1N1) genome having a nucleic acid sequence of SEQ ID NOS: 4-8; and
(ii) genomic segment 1 including a mutated PB2 gene having a nucleic acid sequence of SEQ ID NO:13, or a nucleic acid sequence having at least 75% identity to any one of SEQ ID NO:13;
(iii) genomic segment 2 including a mutated PB1 gene having a nucleic acid sequence of SEQ ID NO:22, or a nucleic acid sequence having at least 75% identity to any one of SEQ ID NO:22; and
(iv) genomic segment 3 including the PA gene of an H7N9 virus having a nucleic acid sequence of SEQ ID NO:3, or
(I)
(i) genomic segments 4-8 of the A/WSN/1933 (H1N1) genome having a nucleic acid sequence of SEQ ID NOS: 4-8; and
(ii) genomic segment 2 including a mutated PB1 gene having a nucleic acid sequence of SEQ ID NO:22, or a nucleic acid sequence having at least 75% identity to any one of SEQ ID NO:22; and
(iii) genomic segments 1 and 3 including the PA gene of an H7N9 virus wherein the virus is completely non replicating in normal human cells.

71-80. (canceled)

81. A vaccine composition for providing immunity to influenza viruses in a subject, including
(i) the intact, replication-deficient virus of claim 1, and
(ii) a pharmaceutically acceptable excipient suitable for intradermal administration, wherein the composition is in an amount effective to induce a protective immune response to one or more influenza viruses in the subject following intradermal administration to the subject.

82. (canceled)

83. The vaccine composition of claim 81, further comprising one or more additional agents selected from group including co-stimulatory molecules, growth factors, adjuvants, and cytokines,
optionally wherein the additional agent is selected from the group including IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Flt3-ligand, and CCR4.

84. (canceled)

85. A method for inducing or stimulating a protective immune response to an influenza virus in a subject, including administering to epidermal tissues of the subject the vaccine composition of claim 81, in an amount effective to induce or stimulate the immune response in the subject,
optionally wherein the vaccine composition is administered to the subject by intradermal injection.

86. (canceled)

87. The method of claim 85, further comprising including administering to the subject one or more additional agents selected from the group including an anti-infective agent, a co-stimulatory molecule, a growth factor, an adjuvant and/or cytokine,
wherein the one or more additional agents are administered before, at the same time, or after administering the vaccine composition,
optionally wherein the additional agent is selected from the group including IL-1, IL-2, IL-7, IL-12, IL-15, IL-18, IL-23, IL-27, B7-2, B7-H3, CD40, CD40L, ICOS-ligand, OX-40L, 4-1BBL, GM-CSF, SCF, FGF, Flt3-ligand, and CCR4.

88-90. (canceled)

91. The method of claim 85, wherein the method provides protective immunity to two or more different strains of influenza viruses,
optionally wherein the method provides protective immunity to
(a) one or more H1N1 influenza viruses and one or more H3N2 influenza viruses; and/or
(b) one or more H5N1 influenza viruses and/or one or more H7N9 influenza viruses.

92-93. (canceled)

94. A kit including the vaccine composition of claim 81 and optionally one or more devices for intradermal administration of the composition to a subject.

95. A dosage unit for immunization by intradermal administration including an effective amount of the vaccine composition of claim 81 for inducing or stimulating a protective immune response to an influenza virus in a subject.

96. A method of making an intact, replication-deficient virus including an influenza virus genome,
wherein the influenza virus genome includes one or more mutations in one or more genes selected from the group including viral RNA polymerase PB1, viral RNA polymerase PB2, viral RNA polymerase PA, and nucleoprotein (NP), including
(a) introducing into a first cell genes encoding 4-7 segments of a wild-type virus; and
(b) introducing into a second cell gene(s) encoding a mutant PB1, PB2, PA, and/or NP, and
(c) co-culturing of the first and second cells; and
(d) isolating the intact, replication-deficient virus.

97. The method of claim 96, wherein
(i) the first and/or second cell is selected from the group including a MDCK cell and a 293FT cell, and/or;
(ii) the mutant PB1, PB2, PA, and/or NP gene(s) is introduced into the cell by lentivirus transduction; and/or
(iii) wherein isolating in step (d) includes purification by
(a) filtration, optionally wherein the filtration includes passing through one or more 0.45 μm filter; or
(b) ultracentrifugation, optionally wherein the ultracentrifugation is sucrose-cushioned ultracentrifugation.

98-105. (canceled)

106. An intact, replication-deficient virus produced according to the method of claim 96.
